(19) United States
(12) Patent Application Publication
(10) Pub. No.: US 2019/0093125 A1
(43) Pub. Date: Mar. 28, 2019

(54) HIGH EFFICIENCY, HIGH THROUGHPUT GENERATION OF GENETICALLY MODIFIED NON-HUMAN MAMMALS BY MULTI-CYCLE ELECTROPORATION OF CAS9 PROTEIN

(71) Applicant: The Jackson Laboratory, Bar Harbor, ME (US)

(72) Inventors: Haoyi Wang, Beijing (CN); Wen-Bo Wang, Bar Harbor, ME (US)

(73) Assignee: The Jackson Laboratory

(21) Appl. No.: 16/034,554

(22) Filed: Jul. 13, 2018

Related U.S. Application Data
(63) Continuation of application No. PCT/US2017/013754, filed on Jan. 17, 2017.
(60) Provisional application No. 62/279,023, filed on Jan. 15, 2016.

(51) Int. Cl. C12N 15/85; C12N 15/11; C12N 9/22; A01K 67/027; A61D 19/04; A61N 1/32

(52) U.S. Cl. CPC C12N 15/8509; C12N 15/11; C12N 9/22; A01K 67/0275; A61D 19/04; A61N 1/327; C12N 2710/22043; C12N 2310/20; A01K 2227/105; A01K 2267/0306; C12N 2015/8536; C12N 2710/16143; C12N 2800/80

(57) ABSTRACT

The invention described herein provides high throughput methods and reagents for generating transgenic animals (e.g., non-human mammals) through introducing a CRISPR/Cas9 system comprising a Cas9 protein into gametes or preimplantation stage embryos (e.g., one-cell embryos or zygotes) via multiple cycles (e.g., 4-10 cycles) of electroporation, leading to genetically inheritable modification to the genome of the animal.

### REFERENCE TO RELATED APPLICATIONS

This application is a continuation of International Patent Application No. PCT/US2017/013754, filed on Jan. 17, 2017, which claims the benefit of the filing date, under 35 U.S.C. § 119(e), of U.S. Provisional Application No. 62/279,023, filed on Jan. 15, 2016.

This application is also related (but does not claim priority) to patent application Ser. No. 15/471,616, filed on Mar. 28, 2017, which is a continuation application of International Application No. PCT/US2015/052928, filed on Sep. 29, 2015, which claims the benefit of the filing date, under 35 U.S.C. § 119(e), of U.S. Provisional Application No. 62/056,687, filed on Sep. 29, 2014.

The entire contents of each of the above-referenced applications are incorporated herein by reference.

### GOVERNMENT SUPPORT

The invention described herein was made with U.S. government support under Grant No. P30CA034196 awarded by the National Cancer Institute of NIH. The U.S. Government has certain rights in the invention.

### BACKGROUND OF THE INVENTION

Delivery of biological or genetic material into mammalian zygote is usually the first step to modify the genome of a mammal, such as the creation of a genetically modified animal model. This is traditionally and presently accomplished by microinjection of the desired biological/genetic material into the zygote by using a glass needle.

Direct microinjection of DNA has been used to introduce Herpes Simplex virus (HSV) TK gene into cultured mammalian cells as early as in late 1980 (Capecchi, Cell, 22:479-488, November 1980). However, the efficiency of transformation was extremely low even when the small pBR 322/TK DNA was co-injected with SV40 DNA, i.e., 15 transformants per 1,000 cells injected. Additionally, the experiment did not result in the production of a mature organism, much less one that could exhibit a phenotypic alteration.

Gordon et al. (Proc. Natl. Acad. Sci. U.S.A., 77:7380-7384, December 1980) first reported that they had successfully microinjected the TK gene (which was incorporated into a chimeric SV40 viral vector) into mice embryos, and had observed such TK genes in mice developed from the microinjected embryos. These mice, however, did not express the TK gene product. That is, in these experiments, no phenotypic alteration of the test animal was accomplished. The results reported by Gordon et al. seems to be consistent with an earlier study by Jaenisch et al. (Proc. Natl. Acad. Sci. U.S.A., 71:1250-1254, 1974) in which the SV40 virus had previously been shown to be functionally and genetically inactive when introduced into mouse embryos.

Subsequently, in June 1981, Wagner and Hoppe filed a U.S. utility application directed to the introduction of exogenous genetic material into embryos through microinjection, which application eventually leads to the issuance of U.S. Pat. No. 4,873,191, a broadly licensed foundation patent in the microinjection field that describes and broadly claims a microinjection method of obtaining a mammal having cells containing and capable of expressing exogenous genetic material.

Despite the numerous improvements since the early 1980s', microinjection remains technically demanding, labor intensive, and time consuming. Successful microinjection, to a great extent, depends on such subjective factors as technical proficiency, training, and experience of the operator. As it requires significant upfront investment in expensive microinjection setup and operators with years of training and experience, the capacity for generating genetically modified mouse models is often limiting even in the best and the most well-funded academic institutions of the world.

In addition, microinjection is inherently low in throughput, requiring manipulating the zygotes one at a time, thus limiting the size and scale of genome engineering experiments.

Electroporation, or electropermeabilization, is a process to significantly increase cell plasma membrane permeability and electrical conductivity, by using an externally applied electrical field. Electroporation has been used in molecular biology as a way of introducing some substances into a cell, such as loading it with a molecular probe, a drug that can change the cell's function, or a DNA molecule.

In molecular biology, the process of electroporation is often used for the transformation of bacteria, yeast, and plant protoplasts. In addition to the lipid membranes, bacteria also have cell walls which are different from the lipid membranes, and are made of peptidoglycan and its derivatives. However, the walls are naturally porous and only act as stiff shells that protect bacteria from severe environmental impacts. If bacteria and plasmids are mixed together, the plasmids can be transferred into the cell after electroporation. Several hundred volts across a distance of several millimeters are typically used in this process.

This procedure can also be used in tissue culture cells, such as cultured mammalian cells. As opposed to transformation in bacteria, the process of introducing foreign DNAs into eukaryotic cells is commonly known as transfection. Electroporation has been used for transfecting cells in suspension using electroporation cuvettes.

Despite the great and obvious need to overcome the many obstacles of microinjection described above, including low efficiency/throughput and technical difficulty, electroporation has not been seen as a viable alternative approach by those skilled in the art, and there has not been reported use of electroporation for introducing genetic materials into mammalian embryos which subsequently develop into genetically modified animals.

Specifically, electroporation has previously been mainly used in tissue culture cells and post-implantation embryos in the mouse (see Mellitzer et al., Mech. Dev., 118:57-63 (2002); Akamatsu et al., Proc. Natl. Acad. Sci. U.S.A., 96:9885-9890 (1999); Potter et al., Proc. Natl. Acad. Sci. U.S.A., 81:7161-7165 (1984); Osumi and Inoue, Methods, 24:35-42 (2001); Fukuchi-Shimogori and Grove, Science 294:1071-1074 (2001); and Tabata and Nakajima, Neuroscience, 103:865-872 (2001)).

In one study, Nemec et al. (Theriogenology 31:233, 1989) described attempts to introduce a neomycin resistant gene (pSVNEO) into mouse 1-cell embryos (i.e., zygotes) through electroporation. However, the DNA comprising the neomycin gene was first injected into the perivitelline space between the plasma membrane and the zona pellucida of the zygotes in order to, according to the authors, decrease the distance between the exogenous DNA and the pronuclei upon exposure to the electrical currents, before the zygotes were subjected to electroporation. The authors reported that, in one experiment, Southern blot analysis revealed no incorporation of the neomycin gene in 55 pups produced from 226 embryos transferred to pseudopregnant ICR recipients. In a follow-up experiment with high salt medium, designed to increase the total energy transferred during the pulses of electroporation, the authors again reported that preliminary data suggest that germ line transmission was not successful.

More recently, electroporation has been used to deliver dsRNA, siRNA, DNA vectors encoding the same, and morpholinos into mouse preimplantation embryos (Grabarek et al., Genesis, 32:269-276 (2002); Wang et al., Dev. Biol., 318:112-125 (2008); and Peng et al., PLoS ONE, 7(8):e43748. doi: 10.1371/journal.pone.0043748 (2012)). However, these reagents work in the cytosol, not in the nucleus, as is required to bring about genetic modification that can be transmitted through germline. More specifically, these reagents work by inhibiting expression of their respective target genes, either by degrading the mRNA of the target gene, or by blocking the translation of the mRNA, or both.

Thus, despite the long-felt need to overcome the many shortcomings of microinjection (such as low efficiency) for use in introducing exogenous biological or genetic material into early stage embryos to generate genetically modified animals through germline transmission, the prevailing art appears to teach away from a seemingly simpler technique such as electroporation.

### SUMMARY OF THE INVENTION

The invention described herein provides methods and reagents for introducing biomolecules into zygotes and early stage (i.e., preimplantation) embryos efficiently. Such technique has wide application in molecular biology. For example, when certain site-directed nucleases (such as the type II CRISPR-Cas9 system) are introduced using the methods of the invention, precise nucleotide changes, large segment deletions, and targeted short segment insertion can be introduced into targeted loci with high efficiency.

One aspect of the invention provides a method of generating a genetically modified mammal (e.g., non-human mammal), the method comprising introducing via electroporation a material (e.g., a CRISPR-Cas system comprising a Type-II Cas9 protein) into a gamete (e.g., an egg of ovum) or a preimplantation stage embryo (e.g., a zygote) of a mammal to modify the genome of the mammal, wherein the material comprises a Type-II Cas9 protein, a Zinc-finger Nuclease (ZFN), a BuD-derived nuclease (BuDN), or a Transcription Activator-Like Effector Nuclease (TALEN), each specific for a target sequence in the genome of the mammal. In certain preferred embodiments, the material comprises a Type-II Cas9 protein.

In a related aspect, the invention provides a method of generating a genetically modified mammal (e.g., non-human mammal), the method comprising introducing via electroporation a material (e.g., a CRISPR-Cas system comprising a Type-II Cas9 protein) into a gamete (e.g., an egg of ovum) or a preimplantation stage embryo (e.g., a zygote) of a mammal to modify the genome of the mammal, wherein: (1) the material comprises a Type-II Cas9 protein, a Zinc-finger Nuclease (ZFN), a BuD-derived nuclease (BuDN), or a Transcription Activator-Like Effector Nuclease (TALEN), each specific for a target sequence in the genome of the mammal; and, (2) the electroporation comprises more than one cycles (e.g., 4, 5, 6, 7, 8, 9, 10 cycles, or 4-10 cycles, preferably 4-8 cycles, or 6 cycles). Although the method of the invention may be useful for gamete (particularly the female gamete such as an unfertilized egg or ovum), in a preferred embodiment, the method is applied to a preimplantation stage embryo that is a zygote.

In yet another related aspect, the invention provides a method of generating a genetically modified mammal (e.g., non-human mammal), the method comprising introducing via electroporation a material (e.g., a CRISPR-Cas system comprising a Type-II Cas9 protein) into a gamete (e.g., an egg of ovum) or a preimplantation stage embryo (e.g., a zygote) of a mammal to modify the genome of the mammal, wherein the electroporation comprises more than one cycles (e.g., 4, 5, 6, 7, 8, 9, 10 cycles, or 4-10 cycles).

In certain embodiments, the electroporation comprises 4-8 cycles, or 6 cycles.

In certain embodiments, some or all cycles of the electroporation are independently carried out at 20-40 volts (e.g., 25-35 volts, or 30 volts, or 25, 26, 27, 28, 29, 30, 31, 32, 33, 34, or 35 volts).

In certain embodiments, some or all cycles of the electroporation are independently carried out using a pulse duration of 1-2 ms (e.g., 1.0 or 1.5 ms).

In certain embodiments, some or all cycles of the electroporation are independently carried out using 1-3 pulses (e.g., 2 pulses).

In certain embodiments, some or all cycles of the electroporation are independently carried out using a pulse interval of 100-1000 ms (e.g., 100 ms, or 125-130 ms).

In certain embodiments, the electroporation consists of 6 cycles, and some or all cycles of said electroporation are carried out using the settings of: 30 volts, 1.0 ms pulse duration, 2 pulses, and 100 ms pulse interval.

In certain embodiments, the material is introduced into the gamete. For example, the gamete may be a female gamete such as an ovum or an egg. In certain embodiments, the method further comprises fusing the electroporated (female) gamete with a gamete of the opposite gender (e.g., a male gamete), and allowing development into a live-born genetically modified mammal.

In certain embodiments, the preimplantation stage embryo is a 1-cell embryo (i.e., a zygote), a 2-cell embryo, a 4-cell embryo, an early morula, or a late morula.

In certain embodiments, the method further comprises allowing the (electroporated) preimplantation stage embryo (e.g., zygote) to develop into a live-born genetically modified mammal.

In certain embodiments, the material comprises a polynucleotide, a polypeptide (e.g., a protein), or both a polynucleotide and a polypeptide. For example, the material to be electroporated may comprise a Type-II Cas9 protein, a Zinc-finger Nuclease (ZFN), a BuD-derived nuclease (BuDN), or a Transcription Activator-Like Effector Nuclease (TALEN), each specific for a target sequence in the genome of the mammal. The material to be electroporated may comprise a coding sequence (e.g., mRNA) for a Type-II Cas9 protein, a Zinc-finger Nuclease (ZFN), a BuD-derived nuclease (BuDN), or a Transcription Activator-Like Effector Nuclease (TALEN). The material to be electroporated may comprise a CRISPR-Cas system guide RNA (e.g., sgRNA) that hybridizes to a target sequence in the genome of the mammal. The material may further comprises a donor polynucleotide. The material may comprise a combination of any of the polynucleotide and polypeptide above. However, in a preferred embodiment, the material comprises a Type-II Cas9 protein, and at least one (e.g., 1, 2, 3, or more) sgRNA each specific for a target sequence in the genome of the mammal.

In certain embodiments, the material comprises a polypeptide comprising a Type-II Cas9 protein. For example, the Cas9 protein can be added to the electroporation mixture at 250 ng/μL final concentration, or 150 ng/μL, 200 ng/μL, 300 ng/μL, or 350 ng/μL final concentration. In certain embodiments, sgRNAs can be added to the electroporation mixture at 300 ng/μL final concentration, or 200 ng/μL, 250 ng/μL, 350 ng/μL, or 400 ng/μL final concentration. In certain embodiments, the Cas9 protein can be added to the electroporation mixture at 250 ng/μL final concentration, and sgRNAs can be added to the electroporation mixture at 300 ng/μL final concentration.

In certain embodiments, the material further comprises a donor polynucleotide. For example, the donor polynucleotide may be a linear or circular double- or single-stranded DNA molecule.

In certain preferred embodiments, the Cas9 protein is electroporated into the gamete (e.g., egg of ovum) or the preimplantation stage embryo (e.g., zygote) with a donor polynucleotide. In certain embodiments, the donor polynucleotide is a linear or circular double stranded DNA molecule.

In certain embodiments, the genome is modified by insertion or deletion of one or more base pairs, by insertion of a heterologous DNA fragment (e.g., the donor polynucleotide), by deletion of an endogenous DNA fragment, by inversion or translocation of an endogenous DNA fragment, or a combination thereof.

In certain embodiments, the genome is modified by non-homologous end joining (NHEJ), homology directed repair (HDR), or homologous recombination (HR).

In certain embodiments, electroporation with Cas9 protein (such as 250 ng/μL) achieves a knock-in (KI) efficiency of at least 50%, 60%, 65%, 70%, 75%, 80%, 85%, 90%, 95% or more (e.g., 100%). In certain embodiments, the KI efficiency is achieved with one cycle of electroporation, or 4, 5, 6, 7, 8, 9, or 10 cycles (preferably 4-10 cycles, such as 6 cycles) of electroporation.

In certain embodiments, electroporation with Cas9 protein (such as 250 ng/μL) achieves a NHEJ efficiency of at least 80%, 85%, 90%, 95%, 97%, or 100%. In certain embodiments, the KI efficiency is achieved with one cycle of electroporation, or 4, 5, 6, 7, 8, 9, or 10 cycles (preferably 4-10 cycles, such as 6 cycles) of electroporation.

In certain embodiments, electroporation with Cas9 protein (such as 250 ng/μL) achieves large target site genomic deletion (e.g., deletion of genomic regions of at least 1 kb, 1.5 kb, 2 kb, 2.5 kb, 3 kb or more) in IVF (in vireo fertilization)-generated embryos at a rate of at least 10%, 15%, 20%, 25%, 30%, 35%, 40% or more. In certain embodiments, the rate is achieved with one cycle of electroporation, or 4, 5, 6, 7, 8, 9, or 10 cycles of electroporation. In certain embodiments, the embryo is one from an inbred mouse strain, such as C57BL/6NJ.

The present invention is partly based on the surprising finding that, when Cas9 protein, instead of Cas9 coding sequence (e.g., mRNA), is delivered through electroporation, results from the analysis of the blastocyst samples indicated that NHEJ and KI efficiency are both extremely high compared to electroporating Cas9 coding sequences.

In certain embodiments, the polynucleotide comprises a coding sequence for a Type-II Cas9 protein.

In certain embodiments, the polynucleotide comprises a Clustered Regularly Interspaced Short Palindromic Repeats (CRISPR)-CRISPR associated (Cas) (CRISPR-Cas) system guide RNA that hybridizes to a target sequence in the genome of the mammal.

In certain embodiments, the concentration ratio for the coding sequence for the Type-II Cas9 protein and the CRISPR-Cas system guide RNA is at least 1:1, at least 1.5:1, at least 2:1, at least 2.5:1, at least 3:1 or higher.

In certain embodiments, the concentration of the coding sequence for the Type-II Cas9 protein is at least 40 ng/μL, at least 50 ng/μL, at least 100 ng/μL, at least 150 ng/μL, at least 200 ng/μL, at least 300 ng/μL, at least 400 ng/μL, at least 500 ng/μL, at least 600 ng/μL, at least 800 ng/μL, or at least 1000 ng/μL or higher.

In certain embodiments, the polynucleotide comprises a coding sequence for a Transcription Activator-Like Effector Nuclease (TALEN) specific for a target sequence in the genome of the mammal.

In certain embodiments, the polynucleotide comprises a coding sequence for a Zinc-finger Nuclease (ZFN) specific for a target sequence in the genome of the mammal.

In certain embodiments, the polynucleotide comprises a coding sequence for a BuD-derived nuclease (BuDN) specific for a target sequence in the genome of the mammal.

In certain embodiments, the polynucleotide comprises a DNA that is randomly integrated into the genome of the mammal.

In certain embodiments, the polynucleotide comprises a DNA vector. In certain embodiments, the polynucleotide comprises a RNA.

In certain embodiments, the polynucleotide comprises a coding sequence for a transposase that integrates a DNA fragment randomly or specifically into the genome of the mammal. In certain embodiments, the polypeptide comprises a transposase.

In certain embodiments, the mammal is a non-human primate (e.g., marmoset, rhesus monkey, chimpanzee), a rodent (e.g., mouse, rat, gerbil, Guinea pig, hamster, cotton rat, naked mole rat), a rabbit, a livestock mammal (e.g., goat, sheep, pig, cow, cattle, horse, camelid), a pet mammal (e.g., dog, cat), a zoo mammal, a marsupial, an endangered mammal, and an outbred or a random bred population thereof. In certain embodiments, the mammal is a mouse or a rat.

In certain embodiments, the gamete (after fusing with a gamete of the opposite gender) or the preimplantation embryo (e.g., the zygote), after electroporation, is transplanted into a pseudopregnant host mammal capable of bearing the embryo to term.

In certain embodiments, the gamete (after fusing with a gamete of the opposite gender) or the preimplantation embryo (e.g., the zygote) is removed from a medium in which electroporation is carried out prior to being transplanted into the pseudopregnant host mammal.

In certain embodiments, more than one, e.g., 2, 3, 5, 10, 20, 30, 40, 50, 100, 125, 150, 200, 250, 300 or more gametes or preimplantation stage embryos, are simultaneously electroporated.

In certain embodiments, at least 50%, 60%, 70%, 80%, 85%, 90%, 95% or more of the electroporated preimplantation stage embryos develop into blastocyst stage embryos, or develop into live-born transgenic mammals.

In certain embodiments, all gametes and preimplantation stage embryos are electroporated in the same electroporation cuvette, such as a 1-mm electroporation cuvette.

In certain embodiments, the gamete or the preimplantation stage embryo is pre-treated to weaken Zona Pellucida prior to electroporation.

In certain embodiments, the gamete or the preimplantation stage embryo is pre-treated in Acidic Tyrode's solution (AT), e.g., for 5-15 seconds, or 10 seconds.

In certain embodiments, the electroporation is carried out using an ECM830 square wave electroporation system (a BTX830 type electroporator).

In certain embodiments, the gamete or the preimplantation stage embryo (e.g., zygote), after electroporation, is frozen for storage. The frozen gamete or the preimplantation stage embryo and be thawed and used in a later time.

In certain embodiments, the method further comprises transferring the electroporated zygote to a pseudopregnant host mammal and allowing the electroporated zygote to develop into a live-born genetically modified mammal.

Another aspect of the invention provided a genetically modified non-human mammal generated by any one of the methods of the invention.

It should be understood that any embodiments described herein above and below are contemplated to be able to combine with any other embodiments of the invention, including those specific embodiments described only in the examples (incorporated herein by reference).

### BRIEF DESCRIPTION OF THE DRAWINGS

FIGS. 1A and 1B show CRISPR-mediated gene disruption in mouse embryos using the subject method. Specifically, FIG. 1A shows genotyping of embryos targeted at the Tet1 locus. RFLP analysis is shown in the top panel and sequencing results are shown in the bottom panel. The restriction site at the target region, used for RFLP analysis, is bold and underlined. The protospacer-adjacent motif (PAM) sequence is colored in red. Mutant alleles from two embryos are shown, and the mutated bases are colored in blue. FIG. 1B shows genotyping of embryos targeted at the Tet2 locus.

FIGS. 2A-2E show CRISPR-mediated gene disruption in live mice using the subject method. Specifically, in FIG. 2A: Cas9 mRNA and sgRNA (single-guide RNA) targeting the Tet2 locus and used at 400 ng/μL and 200 ng/μL, respectively, were delivered to the mouse embryo by electroporation. Electroporated embryos were then transferred to pseudopregnant female mice. Tail biopsy samples were taken from new born mice and genotyped using the RFLP method with EcoRV enzyme. PCR products from mice 85C3 is resistant and 85C10 is partially resistant to EcoRV digestion. In FIG. 2B: Cas9 mRNA and sgRNA targeting the Tet2 locus was used at 600 ng/μL and 300 ng/μL, respectively. PCR products from mice 86C3 and 86C9 were resistant, and those from mice 86C5, 86C7 and 86C8 were partially resistant to EcoRV digestion. In FIGS. 2C-2D: PCR products from mouse 85C3, 85C10, 86C3, 86C5, 86C7, 86C8, and 86C9 were processed by Sanger sequencing and the chromatogram was shown as compared to that from a wild type sample. FIG. 2E: PCR products from mouse 85C3, 86C3 and 86C9 were cloned into pCR2.1-Topo vector, and individual clones were sequenced. INDEL mutations were readily detected among alleles from all 3 founder mice.

FIG. 3A shows results of genotyping of 11 founder mice from Experiment 15. The top panel shows PCR products without RFLP analysis. The middle panel shows RFLP analysis using EcoRV. The bottom panel shows RFLP analysis using EcoRI.

FIG. 3B shows Sanger sequencing results of selected founder mice and control mouse. Changed sequences (through CRISPR/Cas-mediated HDR) in relation to the wild type sequence in the control mouse have been underlined.

FIGS. 4A-4D show that GFP mRNA was efficiently delivered into the mice embryos through multiple cycles of electroporation. In FIG. 4A, the intensity of GFP signal was evaluated after delivery of GFP mRNA through series of electroporation into the mouse embryos. 1, 4, 6 and 8 cycles of electroporation were used for group a, b, c, and d, respectively. FIG. 4B shows the settings of electroporator used for the four groups. In FIG. 4C, embryo survival rate was evaluated after series of electroporation, by counting the embryos in two cell stage or blastocyst stage. In FIG. 4D, GFP signal was quantified at two cell stage after electroporation.

FIGS. 5A-5C show that delivery of Cas9 protein through electroporation dramatically increased the HDR efficiency. FIG. 5A shows schematic of the Aicda target sequence and donor oligonucleotide. The protospacer sequence is underlined and PAM sequence is colored in green. Oligonucleotides changed in the DNA oligonucleotide donor are in uppercase. The BamH1 recognition site is colored in red and the EcoRV recognition site in blue in the DNA oligonucleotide donor. FIG. 5B shows RFLP analysis of 28 mice from groups A, B, C, and D after electroporation. 1, 4, 6 and 8 cycles of electroporation were used in groups A, B, C, and D, respectively. The cleaved bands after EcoRV digestion or BamH1 digestion are indicated by black arrow. FIG. 5C shows sequencing traces of PCR products containing the Aicda target region. Clear sequencing traces indicate there is mainly one allele (defined mutation) in the target locus as shown in B3, C3 and D3, while there are both WT and mutant alleles in A3 mouse. WT, wild type.

FIGS. 6A-6C show that delivery of Cas9 mRNA is less than efficient to generate Aicda genome KI in live mice. FIG. 6A shows RFLP analysis of mice tissue samples from group A, B, and C after electroporation. 1, 4 and 6 times of electroporation were used in group A, B, and C, respectively. The cleaved band after EcoRV digestion is indicated by a black arrow. FIG. 6B shows sequencing traces of PCR products containing the Aicda target region. Overlapping sequencing traces indicate existence of multiple alleles among these mice as shown in C2. In FIG. 6C, PCR product of C2 was cloned and individual clones were sequenced. Sequencing trace from clone C2-8 and is shown.

FIGS. 7A-7C shows that delivery of Cas9 protein through electroporation dramatically increased the HDR efficiency in blastocysts. FIG. 7A shows RFLP analysis of embryos in blastocyst stage after delivery of Cas9 mRNA or Cas9 protein after electroporation. 1, 4, 6 and 8 times of electroporation were performed for the delivery. The cleaved bands after EcoRV or BamH1 digest were indicated by black arrow. FIG. 7B shows sequencing traces of PCR products containing the Aicda target region. Overlapping sequencing traces indicate existence of multiple alleles among these samples as shown in E55 and E67. In FIG. 7C, PCR products were cloned and individual clones were sequenced. Sequences traces from clones E55-1 and E67-1 are shown. WT: wild type. T: tail sample. B: blastocyst.

FIGS. 8A-8C shows that delivery of Cas9 protein through electroporation achieved high efficiency of genome deletion in an inbred strain. FIG. 8A is a schematic of the strategy to delete DNA fragment from the Smc1b gene locus. A DNA segment including Exon2, Exon3 and Exon4 was deleted from the genome. FIG. 8B shows analysis of the sizes of the PCR products through DNA electrophoresis. 1, 4, and 6 times of electroporation were used in group A, B, and C, respectively. A small band in B4, C1, C5, and C6, which is around 518 bp long indicates the successful deletion. FIG. 8C, upper panel, is the Smc1b target sequence. The two protospace sequences of the two gRNAs are labeled in blue and PAM sequences in red. Lower panels, sequencing traces of PCR products containing the Smc1b target region. The blue line and the red line under the WT sequencing trace indicate the upstream gRNA sequence and the PAM sequence respectively. The two blue lines under the sequencing traces of B4, C1, C5, and C6 indicate the remaining upstream and downstream gRNAs. Clear sequencing traces indicate there is mainly one allele in the target locus as shown in B4, C1 and C5. WT: wild type.

FIGS. 9A and 9B show that delivery of Cas9 protein through electroporation achieved high efficiency of large DNA segment deletion in a hybrid strain. FIG. 9A shows analysis of the sizes of the PCR products through DNA electrophoresis. 1, 4 and 6 times of electroporation were used in group A, B, and C, respectively. A small band in A3, A4, A5, B3, C3, and C4, which is around 518 bp long, indicates the successful deletion. FIG. 9B, upper panel, the Smc1b target sequence. The two protospace sequences of the two gRNAs are labeled in blue and PAM sequences in red. Lower panels, sequencing traces of PCR products containing the Smc1b target region. Overlapping sequencing traces indicate existence of multiple alleles among these mice, as shown in A5. Clear sequencing traces indicate there is mainly one allele in the target locus as shown in A3, A4, C3 and C4. WT: wild type.

FIGS. 10A-10C show that delivery of Cas9 protein through electroporation achieved high efficiency of targeted small DNA fragment insertion. FIG. 10A is a schematic of the Rosa26 target sequence and donor oligonucleotide. The protospacer sequence is colored in blue and PAM sequence in red. The LoxP site in the DNA oligonucleotide is underlined. FIG. 10B is a representative image showing the result of analysis of the sizes of the PCR products using TIDE. The red bar at the 34 bp insertion indicates potential insertion of LoxP site. The result of one of the pups with one time of electroporation was shown (A3). In FIG. 10C, PCR products were cloned and individual clones were sequenced. Sequencing trace from clones A3-1 is shown. The successfully inserted LoxP site is underlined.

### DETAILED DESCRIPTION OF THE INVENTION

### 1. Overview

To overcome the inherent limitations associated with microinjection in the context of generating genetically modified animals (e.g., transgenic or other genetically modified mammals), Applicant has developed electroporation-based methodology to deliver biological/genetic materials into animal (e.g., mammalian) gametes or preimplantation embryos including zygotes, under specific conditions that renders the method amenable to high efficiency, high throughput generation of genetically modified animals with high survival rate. The resulting genetic modification can be readily transmitted through germline.

Specifically, in an exemplary embodiment, the methods of the invention were used to deliver CRISPR/Cas system to mouse embryos (e.g., zygotes), thus effecting targeted genetic modification in the genome of the resulting genetically modified/transgenic mice. The methods of the invention, including embodiments or protocols optimized herein, can be carried out using commercially available electroporator, while requiring minimal training or prior experience, and thus are able to process a large number of embryos and multiple gene targeting projects in parallel in a single step. Using the methods of the invention described herein, any of a variety of biological, genetic, or other materials, including but not limited to CRISPR/Cas, TALEN, and ZFN, or combination thereof, etc., can be delivered to gametes and early stage embryos (e.g., mammalian preimplantation embryos including zygotes). Thus the technology described herein enables animal genome engineering with unprecedented ease and scale.

The invention described herein is partly based on the discovery that electroporation can be used to deliver relatively high concentrations (e.g., previously thought to be toxic level) of biological/genetic material into gametes and preimplantation embryos including zygotes, which high concentration may be required to achieve desired end results in the nucleus of the electroporated gametes and early stage embryos, such as genetic modification of the genome of the gametes or embryos, which modification can be passed on via germline transmission.

Specifically, based on prior microinjection experiments, in which relatively low concentrations of Cas9 mRNA (50 or 100 ng/μL) and sgRNA (25 or 50 ng/μL) were successfully used in a pronuclear or cytoplasmic microinjection experiments, Applicant attempted to electroporate zygotes using reported gene-encoding plasmids (see Example 1). Surprisingly, no fluorescence was observed in 3.5-day embryos developed from the electroporated zygotes. Similar experiments using other concentrations of DNA, with pre-treatment of zona pellucida to weaken it; or with other reporters or fluorescein-labeled oligonucleotides, also failed. See Experiments 2-6 mentioned in Example 1.

It is widely believed that the reagents used for the electroporation experiment, including the Cas9 mRNA and the guide RNA, may be toxic to the embryos when used at a higher concentration.

Applicant has demonstrated herein that early stage embryos such as zygotes can be electroporated using relatively high concentrations of biological materials (e.g., polynucleotides comprising CRISPR/Cas mRNA and guide RNA) to effect the desired genetic modifications in embryos or animals developed from the electroporated embryos. See Example 2.

The invention described herein is further partly based on the discovery that electroporated gametes and preimplantation embryos including zygotes usually exhibit retarded or arrested development, or otherwise do not survive to live-born animal, unless the electroporated gametes and preimplantation embryos including zygotes are released into a physiological solution or medium. For example, the solution or medium, among other things, is isotonic. In addition, by releasing the electroporated gametes and preimplantation embryos including zygotes into the physiological solution or medium, the usual electroporation medium is removed (e.g., buffers used to dissolve polynucleotide such as TE), thus further lessening the chance that any harmful substances therein could retard subsequent embryonic development.

For example, when the buffer or medium used for electroporation is not a physiological solution or medium, such as containing excessive (e.g., more than 40% or 50%) buffer used to dissolve materials to be electroporated, it may be helpful to remove such buffer soon after electroporation.

In certain embodiments, when TE or other similar buffer commonly used to dissolve nucleic acid reagents is present in significant amount (e.g., >40% or 50%) in the electroporation medium, several means may be used to mitigate any potential harmful effects. For instance, in one embodiment, the time gametes or embryos are bathed in TE buffer may be minimized by immediately washing the electroporated gametes or embryos in a physiological solution after electroporation. In other embodiment, the nucleic acid reagents (such as the CRISPR/Cas reagents) dissolved in TE buffer can be mixed in proper proportion with a physiological solution such that overall osmolarity is brought closer to physiological. In yet another embodiment, the nucleic acid reagents (e.g., CRISPR/Cas reagents) can be reconstituted directly in a physiological solution to avoid or minimize any complications. That is, the gametes and preimplantation embryos including zygotes may be electroporated in a physiological solution or medium, e.g., one that is isotonic. The physiological solution or medium may be an artificially prepared solution similar to blood plasma in salt composition and osmotic pressure.

Although electroporation can indeed introduce biological material into zygotes under the conditions described herein, survival of the zygotes in subsequent development is required to create live-born animals. Initial failures in culturing electroporated zygotes appear to suggest that zygotes may not survive the electroporation process and continue to develop into live-born animal, regardless of whether the desired genetic modification occurs in the electroporated zygotes. The realization that AT treatment could damage the zygotes excessively to retard embryonic development, and that the reagents delivered, such as the Cas9 mRNA and the guide RNA, could be toxic to the zygotes and their subsequent development, further teaches away from using electroporation as a viable means to permanently introduce germline transmittable genetic modifications in the animal.

Applicant has demonstrated herein that survival rate of the electroporated zygotes, e.g., as measured by successful development into blastocyst stage embryos, can be dramatically improved to surprisingly high levels (e.g., close to or even reaching 100%) using the methods of the invention.

The invention described herein is also partly based on the discovery that using a site specific engineered nuclease, such as a Type-II Cas9 protein, a Zinc-finger Nuclease (ZFN), a BuD-derived nuclease (BuDN), or a Transcription Activator-Like Effector Nuclease (TALEN), directly in electroporation mixture (instead of using coding sequences such as mRNA's therefor), can dramatically increase success rate of the resulting gene editing.

The invention described herein is further partly based on the discovery that carrying out multiple rounds/cycles of electroporation may significantly increase overall efficiency.

Thus one aspect of the invention provides a method of generating a genetically modified animal (e.g., a non-human mammal), the method comprising introducing via electroporation a material (e.g., a CRISPR-Cas system comprising a Type-II Cas9 protein) into a gamete (e.g., an egg or an ovum) or a preimplantation stage embryo (e.g., a zygote) of the animal (e.g., non-human mammal) to modify the genome of the animal (e.g., non-human mammal), wherein: (1) the material comprises a Type-II Cas9 protein, or a Zinc-finger Nuclease (ZFN) or a BuD-derived nuclease (BuDN) or a Transcription Activator-Like Effector Nuclease (TALEN) specific for a target sequence in the genome of the mammal; and/or, (2) said electroporation comprises more than one cycles (e.g., 4, 5, 6, 7, 8, 9, 10 cycles, preferably 4-10 cycles). In a preferred embodiment, the material comprises a CRISPR-Cas system comprising a Type-II Cas9 protein, and/or the electroporation comprises more than one cycles (e.g., 4-10 cycles, or 4 cycles, or 6 cycles).

In a related aspect, the invention also provides a method of generating a genetically modified animal (e.g., a non-human mammal), the method comprising introducing via more than one cycles of electroporation (e.g., 4-10 cycles, 4-8 cycles, or 6 cycles) a material (e.g., a CRISPR-Cas system comprising a Type-II Cas9 protein) into an enucleated oocyte of the animal (e.g., non-human mammal) to modify the genome of the animal (e.g., non-human mammal), wherein a somatic cell is simultaneously fused with the enucleated oocyte by electroporation shock. Fusing a somatic cell with an enucleated oocyte is the method used for pig cloning.

In certain embodiments, some or all cycles of electroporation in the instant invention can be carried out independently under identical or different conditions. In certain embodiments, slightly different electroporation conditions may be used. For example, some or all cycles may be carried out using the settings of: (1) 20-40 volts (e.g., 25-35 volts, or 30 volts, or 25, 26, 27, 28, 29, 30, 31, 32, 33, 34, or 35 volts), (2) pulse duration 1-2 ms (e.g., 1.0 or 1.5 ms), (3) 1-3 pulses (e.g., 2 pulses), and/or (4) pulse interval 100-1000 ms (e.g., 100 ms, or 125-130 ms). In certain embodiments, the electroporation is carried out in a 1-mm electroporation cuvette. In certain embodiments, 2-mm cuvette may also be used with similar parameters with appropriate adjustments.

In certain embodiments, electroporation is carried out in 4, 5, 6, 7, 8, 9, or 10 cycles (e.g., 4-10 cycles, or 6-8 cycles, or 6 cycles), and each cycle is carried out at 30 V, 2 pulses of 1 or 1.5 ms pulse duration each, with 100 ms pulse interval.

In certain embodiments, the material is introduced into the gamete. For example, the gamete may be a sperm (e.g., a mature sperm), a polar body, or an ovum or an egg. If mature sperm is used, the sperm may be first induced to undergo decondensation. Techniques for sperm decondensation are known in the art. See, for example, Mahi et al., J. Reprod. Fert., 44:293-296 (1975); Hendricks et al., Exptl. Cell Res., 40:402-412 (1965); and Wagner et al., Archives of Andrology, 1:31-41 (1978) (all incorporated by reference). Decondensation through the use of a disulfide reductant is preferred. If egg is used, the egg may be in a fertilized or activated state produced by, for example, parthenogenesis. An electroporated polar body may also be used to diploidize a haploid egg.

When gametes are used, the method of the invention further comprises fusing the electroporated gamete with another gamete, such as a gamete of the opposite gender (e.g., fusing an electroporated egg with a sperm), and allowing development of the fused gametes into a live-born genetically modified animal (e.g., a non-human mammal). For example, if a sperm is electroporated, the sperm cell into which genetic modification is effected is thereafter placed in an egg to enable the formation of the zygote. And vice versa. Sperm and egg could both be electroporated before the formation of the zygote.

The electroporated gamete, after fusing with the gamete of the opposite gender or the preimplantation embryo, after electroporation, may be transplanted into a pseudopregnant host animal/mammal, preferably of the same species or strain, which animal (e.g., mammal) is capable of bearing the embryo to term. In certain embodiments, implantation into the pseudopregnant host animal/mammal occurs at the morula or blastocyst stage of development. In other embodiment, implantation occurs immediately after electroporation of the embryo, or immediately after fusing the electroporated gamete with another gamete.

In other embodiments, the material (e.g., a CRISPR-Cas system comprising a Type-II Cas9 protein) is introduced into an early stage embryo, such as a preimplantation stage embryo. The preimplantation stage embryo may be a 1-cell embryo (i.e., a zygote), a 2-cell embryo, a 4-cell embryo, an 8-cell embryo, an early morula, or a late morula. Since embryonic development may sometimes involve asymmetric division, it is possible that, at least transiently, the early state embryo is a 3-, 5-, 6-, 7-cell embryos, or other embryos that are not the result of symmetric embryonic division. The methods of the invention also apply to such early stage embryos. However, in a preferred embodiment, the preimplantation stage embryo is a 1-cell embryo (i.e., a zygote).

In certain embodiments, the method further comprises allowing the (electroporated) preimplantation stage embryo (e.g., zygote) to develop into a live-born genetically modified animal (e.g., non-human mammal). For example, the preimplantation embryo, after electroporation, may be transplanted into a pseudopregnant host animal (e.g., non-human mammal), preferably of the same species or strain, which animal (e.g., non-human mammal) is capable of bearing the embryo to term.

In certain embodiments, the gamete (after fusing with a gamete of the opposite gender) or the preimplantation embryo is first removed from a medium in which electroporation is carried out, prior to being transplanted into the pseudopregnant host animal (e.g., non-human mammal). For example, the electroporated gametes or preimplantation embryos can be washed one or more times in a medium (e.g., an isotonic physiological solution or medium) suitable or compatible for embryonic development. Such wash also removes any substances in the electroporation medium or buffer that may be harmful or otherwise not conducive for further embryonic development in vitro or in vivo.

Suitable medium for this purpose includes, but are not limited to M2-medium or OPTI-MEM® or any other physiological solution (with the appropriate ionic strength and pH). In certain embodiments, the medium or buffer used for washing is pre-warmed to an optimal temperature (e.g., 37° C.) for embryonic development of the animal (e.g., non-human mammal), to reduce or minimize any potential temperature shock.

In certain embodiments, the electroporation medium is isotonic.

In certain embodiments, the electroporated gametes or preimplantation embryos are washed to remove one or more buffers used to dissolve a polynucleotide, such as TE buffer, or an equivalent buffer that contains metal cheaters such as EDTA.

In certain embodiments, the electroporated gametes or preimplantation embryos are washed to remove basal or reduced-serum medium, such as OPTI-MEM® medium (INVITROGEN, Carlsbad, Calif.), siPORT™ (AM8990 or AM8990G; Ambion, Austin, Tex.), or mixture or equivalent thereof, that may be used as electroporation medium.

Numerous reagents or materials may be introduced into the gametes or preimplantation embryos using the methods of the invention, in order to bring about genetic modification that can be transmitted through germline.

In certain embodiments, the material may comprise a polynucleotide, a polypeptide, or both. In a preferred embodiment, the material comprises a CRISPR-Cas system comprising a Type-II Cas9 protein. For example, the material to be electroporated may comprise a Type-II Cas9 protein, a Zinc-finger Nuclease (ZFN), a BuD-derived nuclease (BuDN), or a Transcription Activator-Like Effector Nuclease (TALEN), each specific for a target sequence in the genome of the mammal. The material to be electroporated may comprise a coding sequence (e.g., mRNA) for a Type-II Cas9 protein, a Zinc-finger Nuclease (ZFN), a BuD-derived nuclease (BuDN), or a Transcription Activator-Like Effector Nuclease (TALEN). The material to be electroporated may comprise a CRISPR-Cas system guide RNA (e.g., sgRNA) that hybridizes to a target sequence in the genome of the mammal. The material may further comprises a donor polynucleotide. The material may comprise a combination of any of the polynucleotide and polypeptide above.

In certain embodiments, the material comprises a polypeptide comprising a Type-II Cas9 protein. For example, the Cas9 protein can be added to the electroporation mixture at 250 ng/μL final concentration, or 150 ng/μL, 200 ng/μL, 300 ng/μL, or 350 ng/μL final concentration. In certain embodiments, sgRNAs can be added to the electroporation mixture at 300 ng/μL final concentration, or 200 ng/μL, 250 ng/μL, 350 ng/μL, or 400 ng/μL final concentration. In certain embodiments, the Cas9 protein can be added to the electroporation mixture at 250 ng/μL final concentration, and sgRNAs can be added to the electroporation mixture at 300 ng/μL final concentration.

As used herein, “final concentration” refers to the concentration of the Cas9 protein or sgRNAs, as the case may be, in the mixture that the gamete or preimplantation stage embryo (e.g., zygote) is incubated in the electroporation cuvette.

The present invention is partly based on the surprising finding that, when Cas9 protein, instead of Cas9 coding sequence (e.g., mRNA), is delivered through electroporation, results from the analysis of the blastocyst samples indicated that NHEJ and KI efficiency are both extremely high compared to electroporating Cas9 coding sequences.

In certain embodiments, electroporation with Cas9 protein (such as 250 ng/μL) achieves a knock-in (KI) efficiency of at least 50%, 60%, 65%, 70%, 75%, 80%, 85%, 90%, 95% or more (e.g., 100%). In certain embodiments, the KI efficiency is achieved with one cycle of electroporation, or 4, 5, 6, 7, 8, 9, or 10 cycles (e.g., 4-10 cycles, or 4-8 cycles, or 6 cycles) of electroporation.

In certain embodiments, electroporation with Cas9 protein (such as 250 ng/μL) achieves a NHEJ efficiency of at least 80%, 85%, 90%, 95%, 97%, or 100%. In certain embodiments, the NHEJ efficiency is achieved with one cycle of electroporation, or 4, 5, 6, 7, 8, 9, or 10 cycles (e.g., 4-10 cycles, or 4-8 cycles, or 6 cycles) of electroporation.

In certain embodiments, electroporation with Cas9 protein (such as 250 ng/μL) achieves large target site genomic deletion (e.g., deletion of genomic regions of at least 1 kb, 1.5 kb, 2 kb, 2.5 kb, 3 kb or more) in IVF (in vitro fertilization) generated embryos at a rate of at least 10%, 15%, 20%, 25%, 30%, 35%, 40% or more. In certain embodiments, the rate is achieved with one cycle of electroporation, or 4, 5, 6, 7, 8, 9, or 10 cycles (e.g., 4-10 cycles, or 4-8 cycles, or 6 cycles) of electroporation. In certain embodiments, the embryo is one from an inbred mouse strain, such as C57BL/6NJ.

In other embodiments, additional materials, e.g., those chemical in nature or synthetic such that they promote or inhibit the NHEJ, HDR or HR process, may also be introduced via electroporation. For example, certain chemical reagents may be included to facilitate or enhance the function of the CRISPR/Cas system (see below).

In certain embodiments, the polynucleotide comprises a coding sequence for a Type-II Cas9 protein. In an exemplary embodiment, the coding sequence for the Cas9 protein is an mRNA, such as a codon-optimized mRNA suitable for expression in an eukaryotic cell.

In certain embodiments, the polynucleotide may comprise a Clustered Regularly Interspaced Short Palindromic Repeats (CRISPR)-CRISPR associated (Cas) (CRISPR-Cas) system guide RNA that is capable of hybridization to a target sequence in the genome of the mammal. The guide RNA only needs to be sufficiently similar in sequence to the target sequence such that they can hybridize under intracellular physiological conditions. In certain embodiments, however, the guide RNA is fully complementary (or 100% identical) to the target sequence.

In certain embodiments, the guide RNA may be a guide sequence fused to a trans-activating cr (tracr) sequence.

In certain embodiments, concentration ratio for the coding sequence for the Type-II Cas9 protein and the CRISPR-Cas system guide RNA may vary. For example, suitable ratio may be at least 1:1, at least 1.5:1, at least 2:1, at least 2.5:1, at least 3:1 or higher.

In other embodiments, concentration ratio for the coding sequence for the Type-II Cas9 protein and the CRISPR-Cas system guide RNA may be at most 1:1, at most 1:2, at most 1:4, at most 1:5, at most 1:10 or lower.

In certain embodiments, the concentration of the coding sequence for the Type-II Cas9 protein is at least 40 ng/μL, at least 50 ng/μL, at least 100 ng/μL, at least 150 ng/μL, at least 200 ng/μL, at least 300 ng/μL, at least 400 ng/μL, at least 500 ng/μL, at least 600 ng/μL, at least 800 ng/μL, or at least 1000 ng/μL or higher.

In certain embodiments, the material further comprises a donor polynucleotide (e.g., to facilitate CRISPR-mediated NHEJ, HDR or HR). For example, the donor polynucleotide may be a linear or circular double- or single-stranded DNA molecule. Such donor polynucleotides may be included to facilitate site-specific modification of or around a target sequence which may be the target of the introduced CRISPR-Cas system.

Other than the CRISPR/Cas system, other materials can also be introduced into the gametes or preimplantation embryos using the methods of the invention. For example, in certain embodiments, the polynucleotide may comprise a coding sequence for a Transcription Activator-Like Effector Nuclease (TALEN) specific for a target sequence in the genome of the animal (e.g., mammal). In other embodiments, the polynucleotide may comprise a coding sequence for a Zinc-finger Nuclease (ZFN) specific for a target sequence in the genome of the animal (e.g., mammal). In still other embodiments, the polynucleotide may comprise a coding sequence for a BuD-derived nuclease (BuDN), or modular base-per-base specific nucleic acid binding domains (MBBBD)-derived nuclease, specific for a target sequence in the genome of the animal (e.g., mammal). In yet another embodiment, the polynucleotide comprises a DNA that is randomly integrated into the genome of the animal (e.g., non-human mammal).

The polynucleotide may comprise a DNA vector, or a RNA (e.g., a mRNA, and/or a CRISPR guide RNA).

In certain embodiments, the polynucleotide comprises a transposase that integrates a DNA fragment randomly or specifically into the genome of the mammal.

The present invention has application in the genetic modification of multicellular eukaryotic animals which undergo syngamy, i.e., sexual reproduction by the union of gamete cells. Examples of such animals include amphibians, reptiles, birds, mammals, bony fishes, cartilaginous fishes, cyclostomes, arthropods, insects, mollusks, and thallophytes. Exemplary animals include mammals (such as a non-human mammal), birds, and fishes.

In certain embodiments, the animal is a mammal (see below). For example, the mammal may be a non-human primate (e.g., marmoset, rhesus monkey, chimpanzee), a rodent (e.g., mouse, rat, gerbil, Guinea pig, hamster, cotton rat, naked mole rat), a rabbit, a livestock mammal (e.g., goat, sheep, pig, cow, cattle, horse, camelid), a pet mammal (e.g., dog, cat), a zoo mammal, a marsupial, an endangered mammal, or an individual from an inbred (such as a rat or mouse inbred), an outbred, or a random bred population thereof.

The methods of the invention can be used to introduce numerous germline transmissible modifications to the genome of the animal species involved, including but not limited to: small insertions or deletions (e.g., those of one or more base pairs), insertions of a heterologous DNA fragment, deletions of an endogenous DNA fragment (including large deletions of at least 1, 1.5, 2, 2.5, or 3 kb), inversion or translocation of an endogenous DNA fragment, or a combination thereof. Likewise, numerous different mechanisms may be involved in bringing about the modifications, including but not limited to non-homologous end joining (NHEJ), homology-directed repair (HDR), or homologous recombination (HR).

Homology directed repair (HDR) is a mechanism in cells to repair double strand DNA lesions. This repair mechanism can only be used by the cell when there is a homologue piece of DNA present in the nucleus, mostly in G2 and S phase of the cell cycle.

When the homologue DNA piece is absent, another process called non-homologous end joining (NHEJ) can take place. Non-homologous end joining (NHEJ) is another pathway that repairs double-strand breaks in DNA. NHEJ is referred to as “non-homologous” because the break ends are directly ligated without the need for a homologous template, in contrast to homologous recombination, which requires a homologous sequence to guide repair. NHEJ typically utilizes short homologous DNA sequences (e.g., microhomologies) to guide repair. These microhomologies are often present in single-stranded overhangs on the ends of double-strand breaks. When the overhangs are perfectly compatible, NHEJ usually repairs the break accurately. Imprecise repair leading to loss of nucleotides can also occur, but is much more common when the overhangs are not compatible. Inappropriate NHEJ can lead to translocations and telomere fusion. NHEJ is evolutionarily conserved throughout all kingdoms of life, and is the predominant double-strand break repair pathway in mammalian cells.

When the NHEJ pathway is inactivated, double-strand breaks can be repaired by the more error-prone pathway known as microhomology-mediated end joining (MMEJ). In this pathway, end resection reveals short microhomologies on either side of the break, which are then aligned to guide repair. This contrasts with classical NHEJ, which typically uses microhomologies already exposed in single-stranded overhangs on the DSB ends. Repair by MMEJ therefore leads to deletion of the DNA sequence between the microhomologies.

Homologous recombination is a type of genetic recombination in which nucleotide sequences are exchanged between two similar or identical molecules of homologous DNA. It is most widely used by cells to accurately repair harmful breaks that occur on both strands of DNA, known as double-strand breaks (DSBs). Homologous recombination is also the process that produces new combinations of DNA sequences during meiosis, the process by which eukaryotes make gamete cells, like sperm and egg cells in animals. Homologous recombination is conserved across all three domains of life as well as in viruses.

The methods of the invention are suitable for single gamete or preimplantation embryo (e.g., zygote), and are also suitable for multiplexing and simultaneous use for 2, 3, 5, 10, 20, 30, 40, 50, 100, 125, 150, 200, 250, 300 or more gametes or preimplantation stage embryos (e.g., zygotes), e.g., all can be simultaneously electroporated, either in the same electroporation cuvette, or multiple similar or identical ones, under the same or different conditions. In a preferred embodiment, electroporation is carried out in a 1-mm electroporation cuvette.

The methods of the invention are superior to microinjection in many respects. Compared to microinjection, the subject method is not only much more efficient and much less technically demanding and amenable for standardization, the survival rate of embryos and the rate of germline transmission are both much higher. For example, microinjection can typically achieve a survival rate of 20-25% for injected mice embryos (e.g., 20-25% of the injected mice embryos survive to yield live-born mice pups). In comparison, at least 50%, 60%, 70%, 80%, 85%, 90%, 95% or more of the electroporated preimplantation stage embryos (or electroporated then fused gametes) develop into blastocyst stage embryos, preferably at a rate comparable to a matching control, e.g., in 3.5 days post electroporation for a zygote, or develop into live-born transgenic mammals. For example, the matching control can be sham electroporated gametes or embryos, which may be electroporated by empty vector, scrambled mRNA and/or guide sequence for CRISPR/Cas, or buffer only.

In certain embodiments, at least 70%, 75%, 80%, 85%, 90% or more of the electroporated preimplantation stage embryos (or electroporated then fused gametes) develop into live-born transgenic mammals.

In certain embodiments, targeting efficiency of the host genome, as measured by the percentage of live-born mammals that contain the genetic modification as designed, is at least 80%, 85%, 90%, 95%, 97%, 99%, or close to 100%.

While not wishing to be bound by any particular theory, the high targeting efficiency of the subject method may be owing to the fact that the embryos are submerged in a homogenous CRISPR/Cas solution and upon electroporation, have similar exposure to the CRISPR/Cas reagents. This is in direct contrast to a corresponding microinjection experiment, in which the level of technical competency and factors surrounding a particular experiment may all contribute to relatively large variations in targeting efficiency.

In certain embodiments, the gamete or the preimplantation stage embryo (e.g., zygote) is pre-treated to weaken Zona Pellucida prior to electroporation. For example, the gamete or the preimplantation stage embryo can be pre-treated in Acidic Tyrode's solution (AT) for this purpose, e.g., for 5-15, or 10 seconds.

Tyrode's solution was invented by American pharmacologist Maurice Vejux Tyrode, and is a modification of Ringer-Locke's solution. It is roughly isotonic with interstitial fluid, and is mainly used in physiological experiments and tissue culture. It contains magnesium, a sugar (usually glucose) as an energy source, and uses bicarbonate and/or HEPES as a buffer. Some variations also include phosphate and sulfate ions. It is typically gassed with 95% oxygen 5% carbon dioxide when used for cell culture applications and physiology experiments.

Acidic Tyrode's solution can typically be prepared by dissolving in 100 mL of water the following at room temperature and adjusting to pH 2.5 with Analar HCl (BDH): NaCl, 0.8 g; KCl, 0.02 g; CaCl₂.2H₂O, 0.024 g; MgCl₂.6H₂O, 0.01 g; Glucose, 0.1 g; Polyvinylpyrrolidone (PVP), 0.4 g. The PVP is added to increase viscosity and reduce embryo stickiness. The solution can be filter-sterilized and stored in aliquots at −20° C. AT is commercially available from venders such as Sigma-Aldrich (e.g., SKU T1788 for 100 mL packages).

In certain embodiments, the gametes or preimplantation embryos (e.g., zygotes) are pre-treated in AT for 5-30 seconds, 5-20 second, or 5-10 seconds. Longer treatment time may also be used if the AT is diluted. Suitable AT treatment time can be empirically determined by treating gametes or preimplantation embryos over different time periods, followed by measuring viability, the percentages of damaged or apoptotic gametes/embryos post treatment, or survival rate of embryos during subsequent development.

In certain embodiments, the electroporated gametes or embryos may be used immediately after electroporation, e.g., fusion with another gamete or transplanting into a pseudopregnant female, respectively. In other embodiments, the gamete or the preimplantation stage embryo, after electroporation, is frozen for storage and then thawed for the next step, e.g., fusion with another gamete or transplanting into a pseudopregnant female, respectively. In yet another embodiment, the electroporated gametes or embryos can first be allowed to develop into a later stage embryo, such as an early or late morula, or a blastocyst stage embryo (after first fusing with another gamete in the case of electroporated gamete), before cryopreservation.

Cryopreservation can be done using standard techniques, such as vitrification or slow programmable freezing (SPF).

In certain embodiments, the preimplantation embryos, zygotes, or gametes used for electroporation are obtained by in vitro fertilization (IVF), recovered from cryopreservation.

Another aspect of the invention provides a genetically modified animal (e.g., non-human mammal) generated by any one of the methods of the invention.

With the invention generally described above, the sections below provide additional details about specific aspects of the invention that should be read in combination and in the context of the general description.

Any one of the specific embodiments described herein is contemplated to be combinable with any one or more other embodiments of the invention, including those described only in the Example section.

### 2. Electroporation and Electroporators

Electroporation is a dynamic phenomenon that depends on the local transmembrane voltage at each point on the cell membrane. For a given pulse duration and shape, a specific transmembrane voltage threshold exists for the manifestation of the electroporation phenomenon (e.g., from 0.5 V to 1 V). This leads to the definition of an electric field magnitude threshold for electroporation (Eₜₕ). Only cells within areas where E≥Eₜₕ are electroporated. If a second threshold (Eᵢᵣ) is reached or surpassed, electroporation will compromise the viability of the cells, resulting in irreversible electroporation (IRE).

Electroporation allows cellular introduction of large highly charged molecules, such as DNA, RNA or protein, which may not passively diffuse across the hydrophobic bilayer core. While not wishing to be bound by any particular theory, the mechanism of electroporation is believed to involve the creation of nm-scale water-filled holes in the cell membrane. It is believed that, during electroporation, the lipid molecules in the membrane are not chemically altered, but simply shift position, opening up a pore which acts as the conductive pathway through the bilayer as it is filled with water.

Electroporation is usually a multi-step process with several distinct phases. First, a short electrical pulse must be applied. Typical parameters would be 300-400 mV for <1 ms across the membrane. The voltages used in cell experiments are typically much larger because they are being applied across large distances to the bulk solution so the resulting field across the actual membrane is only a small fraction of the applied bias. Upon application of this potential, the membrane charges like a capacitor through the migration of ions from the surrounding solution. Once the critical field is achieved there is a rapid localized rearrangement in lipid morphology. The resulting structure is believed to be a “pre-pore” since it is not electrically conductive but leads rapidly to the creation of a conductive pore. Evidence for the existence of such pre-pores comes mostly from the “flickering” of pores, which suggests a transition between conductive and insulating states. It has been suggested that these pre-pores are small (˜3 Å) hydrophobic defects. If this theory is correct, then the transition to a conductive state could be explained by a rearrangement at the pore edge, in which the lipid heads fold over to create a hydrophilic interface. Finally, these conductive pores can either heal, resealing the bilayer or expand, eventually rupturing it. The resultant fate depends on whether the critical defect size was exceeded, which in turn depends on the applied field, local mechanical stress, and bilayer edge energy.

According to the instant invention, electroporation of the gametes and preimplantation embryos can be done with commercially available electroporators, appliances that create an electro-magnetic field in the cell solution. Exemplary but non-limiting commercial models of electroporators include: ECM™ 830 Square Wave Electroporator, or 2001 Electro Cell Manipulator (ECM 2001) from BTX (San Diego, Calif.).

In a typical setting, gamete or preimplantation embryo suspension is pipetted into a glass or plastic cuvette, which has two aluminum electrodes on its sides. Prior to electroporation, the gametes or preimplantation embryos are gently mixed with the (biological) materials (e.g., DNA, RNA, or protein, or mixture thereof) to be introduced into the gametes or preimplantation embryos. The mixture can be made either before its pipetting into the cuvette (e.g., a 1-mm or 2-mm width cuvette), or made in the cuvette. Typically, a suspension mixture of around 20 μL total volume is used, although 10-200 μL, 20-100 μL, 25-75 μL total volume may also be used. Then the voltage and capacitance are set (see below), and the cuvette is inserted into the electroporator. Immediately after electroporation, a given amount of (e.g., equal volume as the entire cuvette content, 20-200 μL, or 100 μL) of a liquid medium optimal for embryo survival is added to the cuvette to wash out the electroporated mixture, and the entire content is collected in an appropriate container (e.g., tissue culture dish).

Survival of the electroporated gametes or preimplantation embryos and the development thereof into culture or live-born animal to some extent depends on careful wash and removal of the electroporation medium if it is not a physiological solution. The physiological solution not only provides proper isotonic environment for embryo development, but may also remove any potentially harmful substances that may not be conductive for embryo development/survival.

The gametes or preimplantation embryos, preferably after wash, are transferred to pseudopregnant females or incubated at optimal temperature and/or atmospheric conditions (e.g., at 37° C., 5% CO₂ or 5% O₂/5% CO₂/Nitrogen) for a required period of time before the developing embryos, such as the 3.5-day blastocyst stage embryos, are transferred to surrogate mother/pseudopregnant female to develop to full term and be born as live animal.

A typical electroporation condition, as can be used in the ECM 830 or ECM 2001 model, may include carrying out electroporation in a 1-mm electroporation cuvette, using the settings of: 20-40 volts (e.g., 25-35 volts, or 30 volts, or 25, 26, 27, 28, 29, 30, 31, 32, 33, 34, or 35 volts), pulse duration 1-2 ms (e.g., 1.0 or 1.5 ms), 1-3 pulses (e.g., 2 pulses), pulse interval 100-1000 ms (e.g., 100 ms, or 125-130 ms). In certain embodiments, electroporation is carried out in 4, 5, 6, 7, 8, 9, or 10 cycles (or 6-8 cycles), and each cycle is carried out at 30 V, 2 pulses of 1 or 1.5 ms pulse duration each, with 100 ms pulse interval.

However, it should be understood that the conditions disclosed herein are for illustrative purpose and are not limiting. One of skill in the art can readily adjust the parameters based on, for example, the size and amount of the molecules to be introduced, the type of gametes or embryos, etc.

Electroporation can be done in commercially available bench top electroporators, such as the ECM830 or ECM 2001 model from BTX, and the NUCLEOFECTOR® Devices (Lonza Group Ltd.). Some units offer the possibility of electroporating multiple samples at the same time with special electrode assemblies that fit into multi-well cell culture dishes. Bench top electroporators can also be set to different operating parameters, allowing operators to explore or optimize specific parameters, such as field strengths.

### 3. Mammals

Other than the multicellular eukaryotic animals which undergo syngamy, as described above, the methods of the invention are particularly suitable for any mammals, including human, non-human primates (e.g., marmosets, rhesus monkey, chimpanzees), rodents (e.g., mice, rats, gerbils, Guinea pigs, hamsters, cotton rats, naked mole rats), rabbits, livestock mammals (e.g., goat, sheep, pigs, cows, cattle, horses, camelids), pet mammals (e.g., dogs, cats), zoo mammals, marsupials, endangered mammals, and all outbred or random bred populations thereof.

In certain embodiments, the mammal is a mouse, such as an inbred mouse.

Exemplary inbred mouse strains include, without limitation, inbred mouse lines C3H, e.g., CBA, DBA, FVB, NOD, BALB/c, 129, C57BL, and C57BL mice including C57BL/6J, C57BL/6NTac, C57BL/6NCrl, C57BL/10, etc.

Other inbred mouse strains, including recombinant inbred strains, include (without limitation): 129S1/SvImJ, 129T2/SvEmsJ, 129X1/SvJ, 129P3/J, A/J, AKR/J, BALB/cBy, BALB/cByJ, BALB/c BALB/cJ, C3H/HeJ, C3H/HeOuJ, C3HeB/FeJ, C57BL/10J, C58, CBA/CaHN-Btkxid/J, CBA/J, DBA/1J, DBA/1LacJ, DBA/2J, FVB/NJ, MRL/MpJ, NOD/LtJ, SJL/J, SWR/J, NOR/LtJ, NZB/B1NJ, NZW/LacJ, RBF/DnJ, 129S6/SvEvTac, AJTAC, BALB/cAnNTac, BALB/cJBomTac, BALB/cABomTac, C57BL/6NTac, C57BL/6JBomTac, C57BL/10SgAiTac, C3H/HeNTac, CBA/JBomTac, DBA/1JBomTac, DBA/2NTac, DBA/2JBomTac, FVB/NTac, NOD/MrkTac, NZM/AegTac, SJL/JcrNTac, BALB/cAnNCrlBR, C3H/HeNCrlBR, C57BL/6NCrlBR, DBA/2NCrlBR, FVB/NCrlBR, C. B-17/IcrCrlBR, 129/SvPasIcoCrlBR, SJL/JorlIcoCrlBR, A/JolaHsd, BALB/cAnNHsd, C3H/HeNHsd, C57BL/10ScNHsd, C57BL/6NHsd, CBA/JCrHsd, DBA/2NHsd, FVB/NHsd, SAMP1/KaHsd, SAMP6/TaHsd, SAMP8/TaHsd, SAMP10/TaHsd, SJL/JCrHsd, AKR/OlaHsd, BiozziABH/RijHsd, C57BL/6JOlaHsd, FVB/NhanHsd, MRL/MpOlaHsd, NZB/OlaHsd, NZW/OlaHsd, SWR/OlaHsd, 129P2/OlaHsd, and 129S2/SvHsd, B6.129P2-Apoetm1Unc/J, NOD. CB17-Prkdcscid/J, 129S1/SvImJ, 129X1/SvJ, B10. A-H2a H2-T18a/SgSnJ, B10.D2-Hc0 H2d H2-T18c/oSnJ, B10.D2-Hc1 H2d H2-T18c/nSnJ, B10.RIII-H2r H2-T18b/(71NS)SnJ, B6(C)—H2-Ab1bm12/KhEgJ, B6.129P2-I110tm1Cgn/J, B6.129P2-Nos2tm1Lau/J, B6.129P2-Nos3tm1Unc/J, B6.129S2-Cd8atm1Mak/J, B6.129S2-Igh-6tm1Cgn/J, B6.129S7-Ifngtm1Ts/J, B6.129S7-Rag1tm1Mom/J, B6.CB17-Prkdcscid/SzJ, B6.MRL-Fas1pr/J, B6.V-Lepob/J, BKS.Cg-m+/+Leprdb/J, C3HeB/FeJ, C57BL/10J C57BL/10SnJ, C57BL/6-Tg(APOA1)1Rub/J, C57BL/6J-Tyrc-2J/J, CBA/CaHN-Btkxid/J, CBA/CaJ, CBySmn.CB17-Prkdcscid/J, FVB/N-Tg(MMTVneu)202Mul/J, KK.Cg-Ay/J, MRL/MpJ, MRL/MpJ-Faslpr/J, SJL/J, SWR/J, B10.PL-H2u H2-T18a/(73NS)SnJ, NONcNZO5/LtJ, WR, BPH/2, BPL/1, FS, P/J, P/A, and PRO.

In certain embodiments, the mouse also includes variants with all genetically engineered (e.g. transgenic, knockout, knockin, knockdown, retroviral, viral) or chemically induced mutations (e.g., ENU, EMS also including archives of mutants); radiation induced mutations; spontaneous mutations/modifications maintained on mouse strains, particularly mutants derived from, for example, C57BL/6, 129, FVB, C3H, NOD, DBA/2, BALB/c, and CD-1, including congenic strains and recombinant congenic strains. Specific examples include: B6.129P2-Apoetm1Unc/J, B6.129S4-Pdyn^{tm1Ute}/J, B6; 129P2-Pemt^{tm1J}/J, NOD.Cg Prkdc^{scid}-B2 m^{b}/Dvs, etc.

In certain embodiments, the mouse is a mouse hybrid strain produced by crossing two inbred strains, including mixed inbred strains. Exemplary hybrid strain includes: NZBWF1/J, B6CBAF1/J, B6SJLF1/J, CB6F1/J, CByB6F1/J, PLSJLF1/J, WBB6F1/J-KitW/KitW-v, B6129PF1/J, CAF1/J, B6129PF2/J, B6129SF1/J, B6129SF2/J, B6AF1/J, B6C3F1/J, B6CBAF1/J, and B6SJLF1/J.

In any of the hybrid strains, the proportions may vary from 50:50 F1 to 99:1 F1.

In certain embodiments, the mouse is an outbred mouse, such as CD-1; ICR; Black Swiss; Swiss Webster; NIH Swiss; CF-1, and Nude mouse.

In certain embodiments, the rat is an inbred rat, such as ACI, Brown Norway (BN), BCIX, Copenhagen (COP), MWF, D Agouti (DA), Goto-Kakizaki (GK), Lewis, Fischer 344 (F344), Wistar Furth (WF), Wistar Kyoto (WKY; WKYN1), or ZDF.

In certain embodiments, the rat also includes variants with all genetically engineered (e.g. transgenic, knockout, knockin, knockdown, retroviral, viral) or chemically induced mutations (e.g., ENU, also including archives of mutants); radiation-induced mutations; spontaneous mutations/modifications maintained on rat strains including congenic strains. Specific examples include: F344/NTac-Tg(HLA-B27)-Tg(2M)33-3Trg; HsdAmc:TR-Abcc2; HsdHlr:ZUCKER-Leprfa; and NIH Nude.

In certain embodiments, the rat is a rat hybrid strain produced by crossing two inbred strains. Exemplary hybrid strain includes: BHR, FBNF1/Hsd, and LBNF1/Hsd.

In certain embodiments, the rat is an outbred rat, such as: Holtzman, Sprague Dawley, Long Evans, Wistar, Wistar Han, WH, WKY, Zucker, JCR (Russel Rat), and OFA.

### 4. Genome Modification or Editing Using CRISPR/Cas, TALEN, ZFN, or BuD

The invention described herein provides a powerful tool to modify or “edit” the genome of an animal of interest, such that the modified or edited genome of the animal can be passed along via germline transmission.

More specifically, genome modification or editing may be used to change the genomic sequence of an animal (e.g., mammal) of interest, by, e.g., introducing heterologous transgene or by inhibiting expression of a target endogenous gene. Such genetically modified animals can be used, for example, to establish relevant animal models that correspond to a specific genetic disease or disorder. Such animal models can be used to explore or study disease mechanism, progression, prevention, and treatment (e.g., drug screening and pre-clinical testing).

Illustrative (non-limiting) human diseases or disorders that can be represented in animal models include: various types of cancers, heart diseases, hypertension, metabolic and hormonal disorders, diabetes, obesity, osteoporosis, glaucoma, skin pigmentation diseases, blindness, deafness, neurodegenerative disorders (such as Huntington's or Alzheimer's disease), psychiatric disturbances (including anxiety and depression), and birth defects (such as cleft palate and anencephaly).

Most currently available animal models are made in mice, and they recreate some but may not be all aspects of any particular human disease. For certain disease that may be difficult to mimic using a mouse model, such as many neurodegenerative diseases (most of which involve cognitive deficits), the methods of the invention may be used to generate animal models in non-human primates. For example, a transgenic model of Huntington's disease was recently developed using rhesus macaques that replicated some of the characteristic pathologies of the disorder as it occurs in humans (Yang et al., 2008).

Genome editing may be performed using any art-recognized technology, such as ZFN/TALEN or CRISPR technologies (see review by Gaj et al., Trends in Biotech., 31(7):397-405 (2013), the entire text and all cited references therein are incorporated herein by reference). In a preferred embodiment, genome editing is performed using CRISPR/Cas technology, by electroporating a CRISPR/Cas system comprising a Cas9 protein into a gamete or preimplantation embryo (e.g., a zygote) of a non-human mammal.

Such technologies enable one to manipulate virtually any gene in a diverse range of cell types and organisms, thus enabling a broad range of genetic modifications by inducing DNA double-strand (DSB) breaks that stimulate error-prone nonhomologous end joining (NHEJ) or homology-directed repair (HDR) at specific genomic locations.

Zinc-finger nucleases (ZFNs) and Transcription activator-like effector nucleases (TALENs) are chimeric nucleases composed of programmable, sequence-specific DNA-binding modules linked to a nonspecific DNA cleavage domain. They are artificial restriction enzymes (REs) generated by fusing a zinc-finger or TAL effector DNA binding domain to a DNA cleavage domain. A zinc-finger (ZF) or transcription activator-like effector (TALE) can be engineered to bind any desired target DNA sequence, and be fused to a DNA cleavage domain of an RE, thus creating an engineer restriction enzyme (ZFN or TALEN) that is specific for the desired target DNA sequence. When ZFN/TALEN is introduced into cells, such as a cell of a preimplantation embryo (e.g., a zygote), it can be used for genome editing in situ. Indeed, the versatility of the ZFNs and TALENs can be expanded to effector domains other than nucleases, such as transcription activators and repressors, recombinases, transposases, DNA and histone methyl transferases, and histone acetyltransferases, to affect genomic structure and function.

The Cys₂-His₂ zinc-finger domain is among the most common types of DNA-binding motifs found in eukaryotes and represents the second most frequently encoded protein domain in the human genome. An individual zinc-finger has about 30 amino acids in a conserved ββα configuration. Key to the application of zinc-finger proteins for specific DNA recognition was the development of unnatural arrays that contain more than three zinc-finger domains. This advance was facilitated by the structure-based discovery of a highly conserved linker sequence that enabled construction of synthetic zinc-finger proteins that recognized DNA sequences 9-18 bp in length. This design has proven to be the optimal strategy for constructing zinc-finger proteins that recognize contiguous DNA sequences that are specific in complex genomes. Suitable zinc-fingers may be obtained by modular assembly approach (e.g., using a preselected library of zinc-finger modules generated by selection of large combinatorial libraries or by rational design). Zinc-finger domains have been developed that recognize nearly all of the 64 possible nucleotide triplets, preselected zinc-finger modules can be linked together in tandem to target DNA sequences that contain a series of these DNA triplets. Alternatively, selection-based approaches, such as oligomerized pool engineering (OPEN) can be used to select for new zinc-finger arrays from randomized libraries that take into consideration context-dependent interactions between neighboring fingers. A combination of the two approaches is also used.

Engineered zinc fingers are commercially available. Sangamo Biosciences (Richmond, Calif., USA) has developed a propriety platform (CompoZr) for zinc-finger construction in partnership with Sigma-Aldrich (St. Louis, Mo., USA), which platform allows investigators to bypass zinc-finger construction and validation altogether, and many thousands of proteins are already available. Broadly, zinc-finger protein technology enables targeting of virtually any sequence.

TAL effectors are proteins secreted by the plant pathogenic Xanthomonas bacteria, with DNA binding domain containing a repeated highly conserved 33-34 amino acid sequence, with the exception of the 12th and 13th amino acids. These two locations are highly variable (Repeat Variable Diresidue, or RVD) and show a strong correlation with specific nucleotide recognition. This simple relationship between amino acid sequence and DNA recognition has allowed for the engineering of specific DNA binding domains by selecting a combination of repeat segments containing the appropriate RVDs. Like zinc fingers, modular TALE repeats are linked together to recognize contiguous DNA sequences. Numerous effector domains have been made available to fuse to TALE repeats for targeted genetic modifications, including nucleases, transcriptional activators, and site-specific recombinases. Rapid assembly of custom TALE arrays can be achieved by using strategies include “Golden Gate” molecular cloning, high-throughput solid-phase assembly, and ligation-independent cloning techniques, all can be used in the instant invention for genome editing of the cloned stem cells.

TALE repeats can be easily assembled using numerous tools available in the art, such as a library of TALENs targeting 18,740 human protein-coding genes (Kim et al., Nat. Biotechnol., 31:251-258 (2013)). Custom-designed TALE arrays are also commercially available through, for example, Cellectis Bioresearch (Paris, France), Transposagen Biopharmaceuticals (Lexington, Ky., USA), and Life Technologies (Grand Island, N.Y., USA).

The non-specific DNA cleavage domain from the end of a RE, such as the FokI endonuclease (or FokI cleavage domain variants, such as Sharkey, with mutations designed to improve cleavage specificity and/or cleavage activity), can be used to construct hybrid nucleases that are active in a yeast assay (also active in plant cells and in animal cells). To improve ZFN activity, transient hypothermic culture conditions can be used to increase nuclease expression levels; co-delivery of site-specific nucleases with DNA end-processing enzymes, and the use of fluorescent surrogate reporter vectors that allow for the enrichment of ZFN- and TALEN-modified cells, may also be used. The specificity of ZFN-mediated genome editing can also be refined by using zinc-finger nickases (ZFNickases), which take advantage of the finding that induction of nicked DNA stimulates HDR without activating the error-prone NHEJ repair pathway.

The simple relationship between amino acid sequence and DNA recognition of the TALE binding domain allows for designable proteins. A publicly available software program (DNAWorks) can be used to calculate oligonucleotides suitable for assembly in a two step PCR. A number of modular assembly schemes for generating engineered TALE constructs have also been reported and known in the art. Both methods offer a systematic approach to engineering DNA binding domains that is conceptually similar to the modular assembly method for generating zinc finger DNA recognition domains.

Once the TALEN genes have been assembled, they are introduced into the target cell on a vector using any art recognized methods, such as electroporation or transfection using cationic lipid-based reagents, using plasmid vectors, various viral vectors such as adenoviral, AAV, and Integrase-deficient lentiviral vectors (IDLVs). Alternatively, TALENs can be delivered to the cell as mRNA, which removes the possibility of genomic integration of the TALEN-expressing protein. It can also dramatically increase the level of homology directed repair (HDR) and the success of introgression during gene editing. Finally, direct delivery of purified ZFN/TALEN proteins into cells may also be used. This approach does not carry the risk of insertional mutagenesis, and leads to fewer off-target effects than delivery systems that rely on expression from nucleic acids, and thus may be optimally used for studies that require precise genome engineering in cells, such as the instant stem cells. In any of the embodiments described herein, the TALEN genes, mRNAs or proteins can be introduced into the gametes and preimplantation embryos according to the electroporation methods of the invention.

TALENs can be used to edit genomes by inducing double-strand breaks (DSB), which cells respond to with repair mechanisms. Non-homologous end joining (NHEJ) reconnects DNA from either side of a double-strand break where there is very little or no sequence overlap for annealing. A simple heteroduplex cleavage assay can be run which detects any difference between two alleles amplified by PCR. Cleavage products can be visualized on simple agarose gels or slab gel systems. Alternatively, DNA can be introduced into a genome through NHEJ in the presence of exogenous double-stranded DNA fragments. Homology directed repair can also introduce foreign DNA at the DSB as the transfected double-stranded sequences are used as templates for the repair enzymes. TALENs have been used to generate stably modified human embryonic stem cell and induced pluripotent stem cell (iPSCs) clones to generate knockout C. elegans, rats, and zebrafish.

ZFNs and TALENs are capable of correcting the underlying cause of the disease, therefore permanently eliminating the symptoms with precise genome modifications. For example, ZFN-induced HDR has been used to directly correct the disease-causing mutations associated with X-linked severe combined immune deficiency (SCID), hemophilia B, sickle-cell disease, al-antitrypsin deficiency and numerous other genetic diseases, either by repair defective target genes, or by knocking out a target gene. In addition, these site-specific nucleases can also be used to safely insert a therapeutic transgenes into the subject gametes or preimplantation embryos, at a specific “safe harbor” locations in the target animal genome. Such techniques can be used in gene therapy to “correct” specific genetic defects in a given genome of an animal.

Alternatively, in a preferred embodiment, CRISPR/Cas system can be used to efficiently induce targeted genetic alterations into the subject gametes and preimplantation embryos. CRISPR/Cas (CRISPR associated) systems or “Clustered Regulatory Interspaced Short Palindromic Repeats” are loci that contain multiple short direct repeats, and provide acquired immunity to bacteria and archaea. CRISPR systems rely on crRNA and tracrRNA for sequence-specific silencing of invading foreign DNA. The term “tracrRNA” stands for trans-activating chimeric RNA, which is noncoding RNA that promotes crRNA processing, and is required for activating RNA-guided cleavage by Cas9. CRISPR RNA or crRNA base pairs with tracrRNA to form a two-RNA structure that guides the Cas9 endonuclease to complementary DNA sites for cleavage.

Three types of CRISPR/Cas systems exist: in type II systems, Cas9 serves as an RNA-guided DNA endonuclease that cleaves DNA upon crRNA-tracrRNA target recognition. In bacteria, the CRISPR system provides acquired immunity against invading foreign DNA via RNA-guided DNA cleavage. The CRISPR/Cas system can be retargeted to cleave virtually any DNA sequence by redesigning the crRNA. Indeed, the CRISPR/Cas system has been shown to be directly portable to human cells by co-delivery of plasmids expressing the Cas9 endonuclease and the necessary crRNA components. These programmable RNA-guided DNA endonucleases have demonstrated multiplexed gene disruption capabilities and targeted integration in iPS cells, and can thus be used similarly in the subject methods.

Any CRISPR/Cas system, including the associated Cas proteins and RNA guide sequences, as well as their coding sequences (e.g., mRNA for Cas9 or coding sequence for the guide RNA) or vectors encompassing such coding sequences can be used with the methods of the invention. See those described in US 2014-0068797 A1 and U.S. Pat. No. 8,697,359 (all incorporated herein by reference).

In the description that follows, the RNA guide sequence is also referred to as “DNA-targeting RNA,” which comprises a targeting sequence and, together with a modifying polypeptide (such as Cas9), provides for site-specific modification of a target DNA and/or a polypeptide associated with the target DNA.

In the description that follows, the Cas9 type of protein in the CRISPR/Cas system is also referred to as “site-specific modifying polypeptides.”

The DNA-targeting RNA directs the activities of an associated polypeptide (e.g., a site-directed modifying polypeptide) to a specific target sequence within a target DNA. A subject DNA-targeting RNA comprises: a first segment (also referred to herein as a “DNA-targeting segment” or a “DNA-targeting sequence”) and a second segment (also referred to herein as a “protein-binding segment” or a “protein-binding sequence”).

The DNA-targeting segment of the DNA-targeting RNA comprises a nucleotide sequence that is complementary to a sequence in a target DNA. The DNA-targeting segment of a subject DNA-targeting RNA interacts with a target DNA in a sequence-specific manner via hybridization (i.e., base pairing). As such, the nucleotide sequence of the DNA-targeting segment may vary and determines the location within the target DNA that the DNA-targeting RNA and the target DNA will interact. The DNA-targeting segment of the DNA-targeting RNA can be modified (e.g., by genetic engineering) to hybridize to any desired sequence within a target DNA.

The DNA-targeting segment can have a length of from 12 nucleotides to 100 nucleotides. For example, the DNA-targeting segment can have a length of from 12 nucleotides (nt) to 80 nt, from 12 nt to 50 nt, from 12 nt to 40 nt, from 12 nt to 30 nt, from 12 nt to 25 nt, from 12 nt to 20 nt, or from 12 nt to 19 nt. For example, the DNA-targeting segment can have a length of from 19 nt to 20 nt, from 19 nt to 25 nt, from 19 nt to 30 nt, from 19 nt to 35 nt, from 19 nt to 40 nt, from 19 nt to 45 nt, from 19 nt to 50 nt, from 19 nt to 60 nt, from 19 nt to 70 nt, from 19 nt to 80 nt, from 19 nt to 90 nt, from 19 nt to 100 nt, from 20 nt to 25 nt, from 20 nt to 30 nt, from 20 nt to 35 nt, from 20 nt to 40 nt, from 20 nt to 45 nt, from 20 nt to 50 nt, from 20 nt to 60 nt, from 20 nt to 70 nt, from 20 nt to 80 nt, from 20 nt to 90 nt, or from 20 nt to 100 nt. The nucleotide sequence (the DNA-targeting sequence) of the DNA-targeting segment that is complementary to a nucleotide sequence (target sequence) of the target DNA can have a length at least 12 nt. For example, the DNA-targeting sequence of the DNA-targeting segment that is complementary to a target sequence of the target DNA can have a length at least 12 nt, at least 15 nt, at least 18 nt, at least 19 nt, at least 20 nt, at least 25 nt, at least 30 nt, at least 35 nt or at least 40 nt. For example, the DNA-targeting sequence of the DNA-targeting segment that is complementary to a target sequence of the target DNA can have a length of from 12 nucleotides (nt) to 80 nt, from 12 nt to 50 nt, from 12 nt to 45 nt, from 12 nt to 40 nt, from 12 nt to 35 nt, from 12 nt to 30 nt, from 12 nt to 25 nt, from 12 nt to 20 nt, from 12 nt to 19 nt, from 19 nt to 20 nt, from 19 nt to 25 nt, from 19 nt to 30 nt, from 19 nt to 35 nt, from 19 nt to 40 nt, from 19 nt to 45 nt, from 19 nt to 50 nt, from 19 nt to 60 nt, from 20 nt to 25 nt, from 20 nt to 30 nt, from 20 nt to 35 nt, from 20 nt to 40 nt, from 20 nt to 45 nt, from 20 nt to 50 nt, or from 20 nt to 60 nt. The nucleotide sequence (the DNA-targeting sequence) of the DNA-targeting segment that is complementary to a nucleotide sequence (target sequence) of the target DNA can have a length at least 12 nt.

In some embodiments, the DNA-targeting sequence of the DNA-targeting segment that is complementary to a target sequence of the target DNA is 20 nucleotides in length. In some embodiments, the DNA-targeting sequence of the DNA-targeting segment that is complementary to a target sequence of the target DNA is 19 nucleotides in length.

The percent complementarity between the DNA-targeting sequence of the DNA-targeting segment and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the DNA-targeting sequence of the DNA-targeting segment and the target sequence of the target DNA is 100% over the seven contiguous 5′-most nucleotides of the target sequence of the complementary strand of the target DNA. In some embodiments, the percent complementarity between the DNA-targeting sequence of the DNA-targeting segment and the target sequence of the target DNA is at least 60% over 20 contiguous nucleotides. In some embodiments, the percent complementarity between the DNA-targeting sequence of the DNA-targeting segment and the target sequence of the target DNA is 100% over the fourteen contiguous 5′-most nucleotides of the target sequence of the complementary strand of the target DNA and as low as 0% over the remainder. In such a embodiments, the DNA-targeting sequence can be considered to be 14 nucleotides in length. In some embodiments, the percent complementarity between the DNA-targeting sequence of the DNA-targeting segment and the target sequence of the target DNA is 100% over the seven contiguous 5′-most nucleotides of the target sequence of the complementary strand of the target DNA and as low as 0% over the remainder. In such a case, the DNA-targeting sequence can be considered to be 7 nucleotides in length.

The protein-binding segment of a subject DNA-targeting RNA interacts with a site-directed modifying polypeptide. The subject DNA-targeting RNA guides the bound polypeptide to a specific nucleotide sequence within target DNA via the above-mentioned DNA-targeting segment. The protein-binding segment of a subject DNA-targeting RNA comprises two stretches of nucleotides that are complementary to one another. The complementary nucleotides of the protein-binding segment hybridize to form a double stranded RNA duplex (dsRNA).

A subject double-molecule DNA-targeting RNA comprises two separate RNA molecules. Each of the two RNA molecules of a subject double-molecule DNA-targeting RNA comprises a stretch of nucleotides that are complementary to one another such that the complementary nucleotides of the two RNA molecules hybridize to form the double stranded RNA duplex of the protein-binding segment.

In some embodiments, the duplex-forming segment of the activator-RNA is at least 60% identical to one of the activator-RNA (tracrRNA) molecules (such as one set forth in SEQ ID NOs: 431-562 of US 2014-0068797 A1, incorporated by reference), or a complement thereof, over a stretch of at least 8 contiguous nucleotides. For example, the duplex-forming segment of the activator-RNA (or the DNA encoding the duplex-forming segment of the activator-RNA) is at least 60% identical, at least 65% identical, at least 70% identical, at least 75% identical, at least 80% identical, at least 85% identical, at least 90% identical, at least 95% identical, at least 98% identical, at least 99% identical, or 100% identical, to one of the tracrRNA sequences set forth in SEQ ID NOs: 431-562 of US 2014-0068797 A1, or a complement thereof, over a stretch of at least 8 contiguous nucleotides.

In some embodiments, the duplex-forming segment of the targeter-RNA is at least 60% identical to one of the targeter-RNA (crRNA) sequences set forth in SEQ ID NOs:563-679 of US 2014-0068797 A1 (incorporated by reference), or a complement thereof, over a stretch of at least 8 contiguous nucleotides. For example, the duplex-forming segment of the targeter-RNA (or the DNA encoding the duplex-forming segment of the targeter-RNA) is at least 65% identical, at least 70% identical, at least 75% identical, at least 80% identical, at least 85% identical, at least 90% identical, at least 95% identical, at least 98% identical, at least 99% identical or 100% identical to one of the crRNA sequences set forth in SEQ ID NOs:563-679 of US 2014-0068797 A1, or a complement thereof, over a stretch of at least 8 contiguous nucleotides.

A two-molecule DNA-targeting RNA can be designed to allow for controlled (i.e., conditional) binding of a targeter-RNA with an activator-RNA. Because a two-molecule DNA-targeting RNA is not functional unless both the activator-RNA and the targeter-RNA are bound in a functional complex with dCas9, a two-molecule DNA-targeting RNA can be inducible (e.g., drug inducible) by rendering the binding between the activator-RNA and the targeter-RNA to be inducible. As one non-limiting example, RNA aptamers can be used to regulate (i.e., control) the binding of the activator-RNA with the targeter-RNA. Accordingly, the activator-RNA and/or the targeter-RNA can comprise an RNA aptamer sequence.

RNA aptamers are known in the art and are generally a synthetic version of a riboswitch. The terms “RNA aptamer” and “riboswitch” are used interchangeably herein to encompass both synthetic and natural nucleic acid sequences that provide for inducible regulation of the structure (and therefore the availability of specific sequences) of the RNA molecule of which they are part. RNA aptamers usually comprise a sequence that folds into a particular structure (e.g., a hairpin), which specifically binds a particular drug (e.g., a small molecule). Binding of the drug causes a structural change in the folding of the RNA, which changes a feature of the nucleic acid of which the aptamer is a part. As non-limiting examples: (i) an activator-RNA with an aptamer may not be able to bind to the cognate targeter-RNA unless the aptamer is bound by the appropriate drug; (ii) a targeter-RNA with an aptamer may not be able to bind to the cognate activator-RNA unless the aptamer is bound by the appropriate drug; and (iii) a targeter-RNA and an activator-RNA, each comprising a different aptamer that binds a different drug, may not be able to bind to each other unless both drugs are present. As illustrated by these examples, a two-molecule DNA-targeting RNA can be designed to be inducible.

Examples of aptamers and riboswitches can be found, for example, in: Nakamura et al., Genes Cells, 2012 May, 17(5):344-364; Vavalle et al., Future Cardiol., 2012 May, 8(3):371-382; Citartan et al., Biosens. Bioelectron., 2012 Apr. 15, 34(1):1-11; and Liberman et al., Wiley Interdiscip. Rev. RNA, 2012 May-June, 3(3):369-384; all of which are herein incorporated by reference in their entirety.

Non-limiting examples of nucleotide sequences that can be included in a two-molecule DNA-targeting RNA include either of the sequences set forth in SEQ ID NOs:431-562 of US 2014-0068797 A1, or complements thereof pairing with any sequences set forth in SEQ ID NOs:563-679 of US 2014-0068797 A1, or complements thereof that can hybridize to form a protein binding segment.

A single-molecule DNA-targeting RNA comprises two stretches of nucleotides (a targeter-RNA and an activator-RNA) that are complementary to one another, are covalently linked by intervening nucleotides (“linkers” or “linker nucleotides”), and hybridize to form the double stranded RNA duplex (dsRNA duplex) of the protein-binding segment, thus resulting in a stem-loop structure. The targeter-RNA and the activator-RNA can be covalently linked via the 3′ end of the targeter-RNA and the 5′ end of the activator-RNA. Alternatively, targeter-RNA and the activator-RNA can be covalently linked via the 5′ end of the targeter-RNA and the 3′ end of the activator-RNA.

The linker of a single-molecule DNA-targeting RNA can have a length of from 3 nucleotides to 100 nucleotides. For example, the linker can have a length of from 3 nucleotides (nt) to 90 nt, from 3 nucleotides (nt) to 80 nt, from 3 nucleotides (nt) to 70 nt, from 3 nucleotides (nt) to 60 nt, from 3 nucleotides (nt) to 50 nt, from 3 nucleotides (nt) to 40 nt, from 3 nucleotides (nt) to 30 nt, from 3 nucleotides (nt) to 20 nt or from 3 nucleotides (nt) to 10 nt. For example, the linker can have a length of from 3 nt to 5 nt, from 5 nt to 10 nt, from 10 nt to 15 nt, from 15 nt to 20 nt, from 20 nt to 25 nt, from 25 nt to 30 nt, from 30 nt to 35 nt, from 35 nt to 40 nt, from 40 nt to 50 nt, from 50 nt to 60 nt, from 60 nt to 70 nt, from 70 nt to 80 nt, from 80 nt to 90 nt, or from 90 nt to 100 nt. In some embodiments, the linker of a single-molecule DNA-targeting RNA is 4 nt.

An exemplary single-molecule DNA-targeting RNA comprises two complementary stretches of nucleotides that hybridize to form a dsRNA duplex. In some embodiments, one of the two complementary stretches of nucleotides of the single-molecule DNA-targeting RNA (or the DNA encoding the stretch) is at least 60% identical to one of the activator-RNA (tracrRNA) molecules set forth in SEQ ID NOs:431-562 of US 2014-0068797 A1, or a complement thereof, over a stretch of at least 8 contiguous nucleotides. For example, one of the two complementary stretches of nucleotides of the single-molecule DNA-targeting RNA (or the DNA encoding the stretch) is at least 65% identical, at least 70% identical, at least 75% identical, at least 80% identical, at least 85% identical, at least 90% identical, at least 95% identical, at least 98% identical, at least 99% identical or 100% identical to one of the tracrRNA sequences set forth in SEQ ID NOs:431-562 of US 2014-0068797 A1, or a complement thereof, over a stretch of at least 8 contiguous nucleotides.

In some embodiments, one of the two complementary stretches of nucleotides of the single-molecule DNA-targeting RNA (or the DNA encoding the stretch) is at least 60% identical to one of the targeter-RNA (crRNA) sequences set forth in SEQ ID NOs:563-679, or a complement thereof, over a stretch of at least 8 contiguous nucleotides. For example, one of the two complementary stretches of nucleotides of the single-molecule DNA-targeting RNA (or the DNA encoding the stretch) is at least 65% identical, at least 70% identical, at least 75% identical, at least 80% identical, at least 85% identical, at least 90% identical, at least 95% identical, at least 98% identical, at least 99% identical or 100% identical to one of the crRNA sequences set forth in SEQ ID NOs:563-679 of US 2014-0068797 A1, or a complement thereof, over a stretch of at least 8 contiguous nucleotides.

Appropriate naturally occurring cognate pairs of crRNAs and tracrRNAs can be routinely determined for SEQ ID NOs:431-679 of US 2014-0068797 A1 by taking into account the species name and base-pairing (for the dsRNA duplex of the protein-binding domain) when determining appropriate cognate pairs.

With regard to both a single-molecule DNA-targeting RNA and to a double-molecule DNA-targeting RNA, artificial sequences that share very little (roughly 50% identity) with naturally occurring a tracrRNAs and crRNAs can function with Cas9 to cleave target DNA as long as the structure of the protein-binding domain of the DNA-targeting RNA is conserved. Thus, RNA folding structure of a naturally occurring protein-binding domain of a DNA-targeting RNA can be taken into account in order to design artificial protein-binding domains (either two-molecule or single-molecule versions). As a non-limiting example, functional artificial DNA-targeting RNA can be designed based on the structure of the protein-binding segment of the naturally occurring DNA-targeting (e.g., including the same number of base pairs along the RNA duplex and including the same “bulge” region as present in the naturally occurring RNA). As structures can readily be produced by one of ordinary skill in the art for any naturally occurring crRNA:tracrRNA pair from any species (see SEQ ID NOs:431-679 of US 2014-0068797 A1 for crRNA and tracrRNA sequences from a wide variety of species), an artificial DNA-targeting-RNA can be designed to mimic the natural structure for a given species when using the Cas9 (or a related Cas9) from that species. Thus, a suitable DNA-targeting RNA can be an artificially designed RNA (non-naturally occurring) comprising a protein-binding domain that was designed to mimic the structure of a protein-binding domain of a naturally occurring DNA-targeting RNA. (see SEQ ID NOs: 431-679 of US 2014-0068797 A1, taking into account the species name when determining appropriate cognate pairs).

The protein-binding segment can have a length of from 10 nucleotides to 100 nucleotides. For example, the protein-binding segment can have a length of from 15 nucleotides (nt) to 80 nt, from 15 nt to 50 nt, from 15 nt to 40 nt, from 15 nt to 30 nt or from 15 nt to 25 nt.

Also with regard to both a single-molecule DNA-targeting RNA and to a double-molecule DNA-targeting RNA, the dsRNA duplex of the protein-binding segment can have a length from 6 base pairs (bp) to 50 bp. For example, the dsRNA duplex of the protein-binding segment can have a length from 6 bp to 40 bp, from 6 bp to 30 bp, from 6 bp to 25 bp, from 6 bp to 20 bp, from 6 bp to 15 bp, from 8 bp to 40 bp, from 8 bp to 30 bp, from 8 bp to 25 bp, from 8 bp to 20 bp or from 8 bp to 15 bp. For example, the dsRNA duplex of the protein-binding segment can have a length from 8 bp to 10 bp, from 10 bp to 15 bp, from 15 bp to 18 bp, from 18 bp to 20 bp, from 20 bp to 25 bp, from 25 bp to 30 bp, from 30 bp to 35 bp, from 35 bp to 40 bp, or from 40 bp to 50 bp. In some embodiments, the dsRNA duplex of the protein-binding segment has a length of 36 base pairs. The percent complementarity between the nucleotide sequences that hybridize to form the dsRNA duplex of the protein-binding segment can be at least 60%. For example, the percent complementarity between the nucleotide sequences that hybridize to form the dsRNA duplex of the protein-binding segment can be at least 65%, at least 70%, at least 75%, at least 80%, at least 85%, at least 90%, at least 95%, at least 98%, or at least 99%. In some cases, the percent complementarity between the nucleotide sequences that hybridize to form the dsRNA duplex of the protein-binding segment is 100%.

A DNA-targeting RNA and a site-directed modifying polypeptide form a complex. The DNA-targeting RNA provides target specificity to the complex by comprising a nucleotide sequence that is complementary to a sequence of a target DNA (as noted above). The site-directed modifying polypeptide of the complex provides the site-specific activity. In other words, the site-directed modifying polypeptide is guided to a DNA sequence (e.g. a chromosomal sequence or an extrachromosomal sequence, e.g. an episomal sequence, a minicircle sequence, a mitochondrial sequence, a chloroplast sequence, etc.) by virtue of its association with at least the protein-binding segment of the DNA-targeting RNA.

A site-directed modifying polypeptide modifies target DNA (e.g., cleavage or methylation of target DNA) and/or a polypeptide associated with target DNA (e.g., methylation or acetylation of a histone tail). A site-directed modifying polypeptide is also referred to herein as a “site-directed polypeptide” or an “RNA binding site-directed modifying polypeptide.”

In some embodiments, the site-directed modifying polypeptide is a naturally-occurring modifying polypeptide. In other cases, the site-directed modifying polypeptide is not a naturally-occurring polypeptide (e.g., a chimeric polypeptide as discussed below or a naturally-occurring polypeptide that is modified, e.g., mutation, deletion, insertion).

Exemplary naturally-occurring site-directed modifying polypeptides are set forth in SEQ ID NOs:1-255 of US 2014-0068797 A1 (incorporated by reference) as a non-limiting and non-exhaustive list of naturally occurring Cas9/Csn1 endonucleases. These naturally occurring polypeptides, as disclosed herein, bind a DNA-targeting RNA, are thereby directed to a specific sequence within a target DNA, and cleave the target DNA to generate a double strand break.

A site-directed modifying polypeptide comprises two portions, an RNA-binding portion and an activity portion. In some embodiments, a site-directed modifying polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that exhibits site-directed enzymatic activity (e.g., activity for DNA methylation, activity for DNA cleavage, activity for histone acetylation, activity for histone methylation, etc.), wherein the site of enzymatic activity is determined by the DNA-targeting RNA.

In other embodiments, a site-directed modifying polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that modulates transcription within the target DNA (e.g., to increase or decrease transcription), wherein the site of modulated transcription within the target DNA is determined by the DNA-targeting RNA.

In some embodiments, a site-directed modifying polypeptide has enzymatic activity that modifies target DNA (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In other cases, a subject site-directed modifying polypeptide has enzymatic activity that modifies a polypeptide (e.g., a histone) associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Exemplary site-directed modifying polypeptides are further provided below.

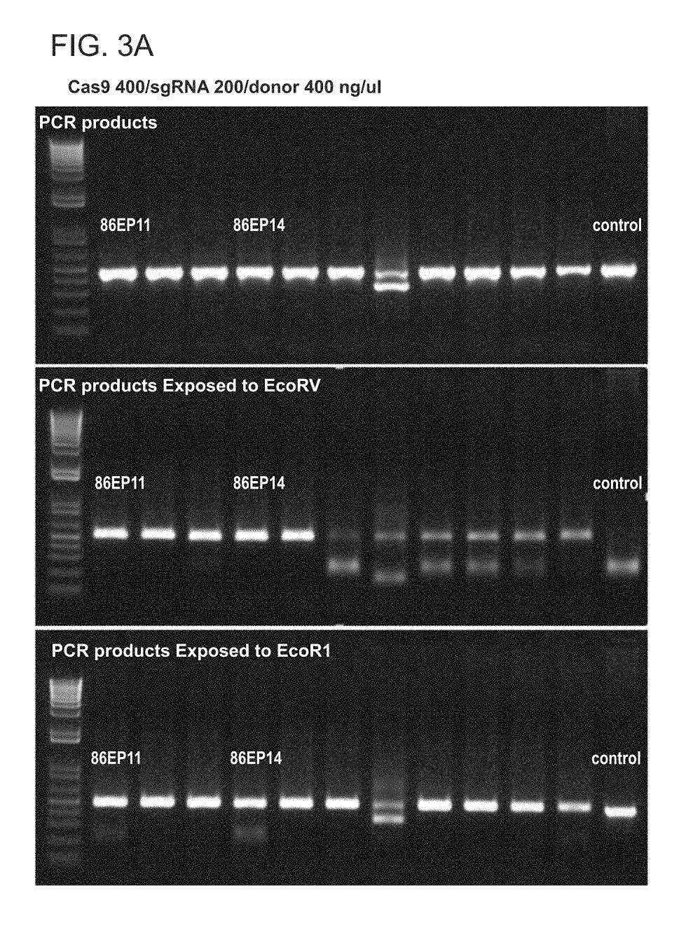

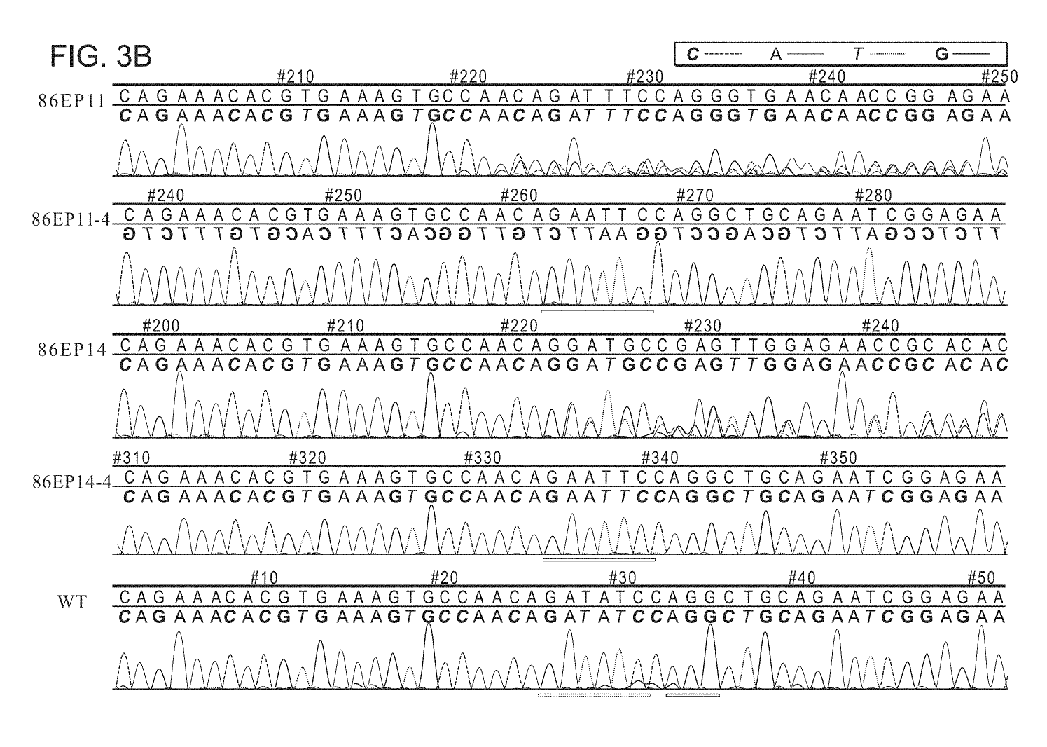

Specifically, in some embodiments, the site-directed modifying polypeptide comprises an amino acid sequence having at least 75%, at least 80%, at least 85%, at least 90%, at least 95%, at least 99%, or 100%, amino acid sequence identity to amino acids 7-166 or 731-1003 of the Cas9/Csn1 amino acid sequence depicted in FIG. 3 of US 2014-0068797 A1, or to the corresponding portions in any of the amino acid sequences set forth as SEQ ID NOs:1-256 and 795-1346 of US 2014-0068797 A1 (all incorporated by reference).

In some embodiments, a nucleic acid (e.g., a DNA-targeting RNA) comprises one or more modifications, e.g., a base modification, a backbone modification, etc., to provide the nucleic acid with a new or enhanced feature (e.g., improved stability). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Examples of suitable nucleic acids containing modifications include nucleic acids containing modified backbones or non-natural internucleoside linkages. Nucleic acids (having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage, i.e., a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240. Both incorporated by reference.

Also suitable are nucleic acids having morpholino backbone structures as described in, e.g., U.S. Pat. No. 5,034,506 (incorporated by reference). For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A subject nucleic acid can also be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262. All incorporated by reference.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 41(14):4503-4510 (2002)). Morpholino-based polynucleotides are disclosed in U.S. Pat. No. 5,034,506 (incorporated by reference). A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 122:8595-8602 (2000), incorporated by reference). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 4:455-456 (1998)). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 97:5633-5638 (2000), incorporated by reference).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 54:3607-3630 (1998), incorporated by reference). LNAs and preparation thereof are also described in WO 98/39352 and WO 99/14226, both incorporated by reference.

A nucleic acid can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁₋₁₀ alkyl or C₂₋₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to 10. Other suitable polynucleotides comprise a sugar substituent group selected from: C₁₋₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH2CH2OCH3, also known as 2′-O-(2-methoxyethyl) or 2′-MOE) (Martin et al., Helv. Chim. Acta, 78:486-504 (1995)) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)2.

Other suitable sugar substituent groups include methoxy (—O—CH₃), aminopropoxy (—OCH₂CH₂CH₂NH₂), allyl (—CH₂—CH═CH₂), —O-allyl (—O—CH₂CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,′:4,5)pyrrolo[2,3-d]pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808 (incorporated by reference), those disclosed in The Concise Encyclopedia of Polymer Science and Engineering, pages 858-859, Kroschwitz, J. I., ed. John Wiley & Sons, 1990; those disclosed by Englisch et al., Angewandte Chemie, International Edition, 1991, 30:613; and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

Another possible modification of a subject nucleic acid involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 86:6553-6556 (1989)); cholic acid (Manoharan et al., Bioorg. Med. Chem. Let., 4:1053-1060 (1994)); a thioether, e.g., hexyl-5-tritylthiol (Manoharan et al., Ann. N.Y. Acad. Sci., 660:306-309 (1992)); Manoharan et al., Bioorg. Med. Chem. Let., 3:2765-2770 (1993)); a thiocholesterol (Oberhauser et al., Nucl. Acids Res., 20:533-538 (1992)); an aliphatic chain, e.g., dodecandiol or undecyl residues (Saison-Behmoaras et al., EMBO J., 10:1111-1118 (1991)); Kabanov et al., FEBS Lett., 259:327-330 (1990)); Svinarchuk et al., Biochimie, 75:49-54 (1993)); a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654 (1995)); Shea et al., Nucl. Acids Res., 18:3777-3783 (1990)); a polyamine or a polyethylene glycol chain (Manoharan et al., Nucleosides & Nucleotides, 14:969-973 (1995)); or adamantane acetic acid (Manoharan et al., Tetrahedron Lett., 36:3651-3654 (1995)); a palmityl moiety (Mishra et al., Biochim. Biophys. Acta, 1264:229-237 (1995)); or an octadecylamine or hexylamino-carbonyl-oxycholesterol moiety (Crooke et al., J. Pharmacol. Exp. Ther., 277:923-937 (1996)); all incorporated by reference.

A conjugate may include a “Protein Transduction Domain” or PTD (also known as a CPP-cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus of an exogenous polypeptide (e.g., a site-directed modifying polypeptide). In some embodiments, a PTD is covalently linked to the carboxyl terminus of an exogenous polypeptide (e.g., a site-directed modifying polypeptide). In some embodiments, a PTD is covalently linked to a nucleic acid (e.g., a DNA-targeting RNA, a polynucleotide encoding a DNA-targeting RNA, a polynucleotide encoding a site-directed modifying polypeptide, etc.). Exemplary PTDs include but are not limited to a minimal undecapeptide protein transduction domain (corresponding to residues 47-57 of HIV-1 TAT comprising YGRKKRRQRRR); a polyarginine sequence comprising a number of arginines sufficient to direct entry into a cell (e.g., 3, 4, 5, 6, 7, 8, 9, 10, or 10-50 arginines); a VP22 domain (Zender et al., Cancer Gene Ther., 9(6):489-496 (2002)); an Drosophila Antennapedia protein transduction domain (Noguchi et al., Diabetes, 52(7):1732-1737 (2003)); a truncated human calcitonin peptide (Trehin et al., Pharm. Research, 21:1248-1256 (2004)); polylysine (Wender et al., Proc. Natl. Acad. Sci. USA, 97:13003-13008 (2000)); RRQRRTSKLMKR; Transportan GWTLNSAGYLLGKINLKALAALAKKIL; KALAWEAKLAKALAKALAKHLAKALAKALKCEA; and RQIKIWFQNRRMKWKK. Exemplary PTDs include but are not limited to, YGRKKRRQRRR, RKKRRQRRR; an arginine homopolymer of from 3 arginine residues to 50 arginine residues; Exemplary PTD domain amino acid sequences include, but are not limited to, any of the following: YGRKKRRQRRR; RKKRRQRR; YARAAARQARA; THRLPRRRRRR; and GGRRARRRRRR. In some embodiments, the PTD is an activatable CPP (ACPP) (Aguilera et al., Integr. Biol. (Camb), 1(5-6):371-381 (June, 2009)). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

Exemplary DNA-Targeting RNAs are further described below.

In some embodiments, a suitable DNA-targeting RNA comprises two separate RNA polynucleotide molecules. The first of the two separate RNA polynucleotide molecules (the activator-RNA) comprises a nucleotide sequence having at least 60%, at least 65%, at least 70%, at least 75%, at least 80%, at least 85%, at least 90%, at least 95%, at least 98%, at least 99%, or 100% nucleotide sequence identity over a stretch of at least 8 contiguous nucleotides to any one of the nucleotide sequences set forth in SEQ ID NOs:431-562, or complements thereof. The second of the two separate RNA polynucleotide molecules (the targeter-RNA) comprises a nucleotide sequence having at least 60%, at least 65%, at least 70%, at least 75%, at least 80%, at least 85%, at least 90%, at least 95%, at least 98%, at least 99%, or 100% nucleotide sequence identity over a stretch of at least 8 contiguous nucleotides to any one of the nucleotide sequences set forth in SEQ ID NOs:563-679 of US 2014-0068797, or complements thereof.

In some embodiments, a suitable DNA-targeting RNA is a single RNA polynucleotide and comprises a first nucleotide sequence having at least 60%, at least 65%, at least 70%, at least 75%, at least 80%, at least 85%, at least 90%, at least 95%, at least 98%, at least 99%, or 100% nucleotide sequence identity over a stretch of at least 8 contiguous nucleotides to any one of the nucleotide sequences set forth in SEQ ID NOs:431-562 of US 2014-0068797, and a second nucleotide sequence having at least 60%, at least 65%, at least 70%, at least 75%, at least 80%, at least 85%, at least 90%, at least 95%, at least 98%, at least 99%, or 100% nucleotide sequence identity over a stretch of at least 8 contiguous nucleotides to any one of the nucleotide sequences set forth in SEQ ID NOs: 463-679 of US 2014-0068797.

In some embodiments, the DNA-targeting RNA is a double-molecule DNA-targeting RNA and the targeter-RNA comprises the sequence 5′GUUUUAGAGCUA-3′ linked at its 5′ end to a stretch of nucleotides that are complementary to a target DNA. In some embodiments, the DNA-targeting RNA is a double-molecule DNA-targeting RNA and the activator-RNA comprises the sequence 5′ UAGCAAGUUAAAAUAAGGCUAGUCCG-3′.

In some embodiments, the DNA-targeting RNA is a single-molecule DNA-targeting RNA and comprises the sequence 5′-GUUUUAGAGCUA-linker-UAGCAAGUUAAAAUAAGGCUAGUCCG-3′ linked at its 5′ end to a stretch of nucleotides that are complementary to a target DNA (where “linker” denotes any a linker nucleotide sequence that can comprise any nucleotide sequence). Other exemplary single-molecule DNA-targeting RNAs include those set forth in SEQ ID NOs: 680-682 of US 2014-0068797 (incorporated by reference).

In certain embodiments, the site-specific modifying polypeptides (e.g., Cas9) may be constitutively or conditionally (e.g., tissue- or cell type-specifically or inducibly) expressed as a transgene in an animal of interest, such as a mouse or a rat, such that only the DNA-targeting RNA or guide sequence needs to be introduced into gametes or preimplantation embryos obtained from such animals to provide a functional CRISPR/Cas system in the gametes or preimplantation embryos. For example, see the Cre-dependent Cas9 knockin mouse described in Platt et al., RISPR-Cas9 Knockin Mice for Genome Editing and Cancer Modeling, Cell http://dx dot doi dot org slash 10.1016 slash j dot cell dot 2014.09.014 (incorporated by reference), in which successful genome editing was demonstrated in neurons, immune cells, and endothelial cells, using guide RNA delivered by adeno-associated virus (AAV)-, lentivirus-, or particle-mediated delivery methods. The Cas9 type of transgene can be driven by, for example, a ubiquitous promoter (such as the CAG promoter), and may be rendered inducible by using the Cre-loxP system (e.g., with cell type- or tissue-specific Cre drivers) or equivalents or variants thereof well known in the art.

In certain embodiments, the Cas9-expressing animal is fertile, has normal litter sizes, presents no morphological abnormalities, and/or is able to breed to homozygosity.

Such site-specific modifying polypeptide-expressing animals (e.g., mouse or rat), coupled with the high efficiency, high throughout electroporation methods of the invention, may be used in in vivo high-throughput genetic screen to, for example, identify genes involved in any of many important biological processes such as tumor suppression, stem-cell renewal, host determinants of virus replication, and regulators of oncogenic growth, and may be directly combined with genome-wide or targeted sgRNA library to uncover novel biology.

### Examples

The examples described herein are for illustrative purpose only, and are not limiting. The conditions described in the examples, however, may have general applicability and constitute specific embodiments of the described invention that are contemplated to be combinable with any one or more other embodiments of the invention described herein.

Genetically modified mice serve as important models for studying gene function and human diseases, and predominantly have been created using conventional gene targeting or transgenic technologies. In a typical conventional gene targeting experiment, the desired genetic modification is first introduced into the mouse embryonic stem (ES) cells through homologous recombination, and the clonally derived ES cell clones carrying the intended mutation are isolated (Capecchi, 2005). Targeted ES cells are subsequently injected into wild-type blastocysts. Some of the targeted ES cells may contribute to the germline of the resulting chimeric animals, thus generating mice containing the targeted gene modification (Capecchi, 2005).

Although effective, gene targeting is costly and time-consuming, and heavily relies on the availability and performance of a germline competent ES cell line.

To circumvent the need of a germline competent ES cell line, alternative methodologies have been developed using site-specific DNA endonucleases, including zinc-finger nucleases (ZFNs) (Urnov et al., 2010) and transcription activator-like effector nucleases (TALENs) (Bogdanove and Voytas, 2011), which could be injected directly into the one-cell embryos to generate animals with specific gene disruption (Carbery et al., 2010; Geurts et al., 2009; Sung et al., 2013; Tesson et al., 2011). When a single stranded DNA (ssDNA) or a plasmid with homology flanking the double strand break site (DSB) is provided, a defined modification can be introduced into the genome by homologous recombination (HR) (Brown et al., 2013; Cui et al., 2011; Meyer et al., 2010).

Recently, using the RNA-guided clustered regularly interspaced short palindromic repeat (CRISPR)/CRISPR-associated (Cas) nuclease system (Hsu et al., 2014), methods for generation of mice carrying mutations in single and multiple genes, as well as mice carrying reporter and conditional alleles, in one step (Wang et al., 2013; Yang et al., 2013), have been established.

Using a similar approach, genetically modified animals have been generated from various species (Chang et al., 2013; Hai et al., 2014; Hwang et al., 2013; Li et al., 2013a; Li et al., 2013b; Niu et al., 2014). Similar to the traditional approach generating transgenic mice, these studies all employ microinjection to deliver nuclease components into the one-cell zygote.

The methodology described herein uses electroporation to deliver biological/genetic materials, such as the CRISPR/Cas system, to the mammalian (e.g., mouse) zygotes, and is successful to generate live transgenic animals (e.g., mice) carrying targeted gene modifications.

### Example 1 Electroporation of Mouse Zygotes and Presumptive Zygotes with Reporter-Encoding Polynucleotides

This experiment describes the result of electroporating mouse zygotes or presumptive zygotes (“zygotes” for short) with reporter-gene encoding plasmids.

The pMAXGFP vector (Lonza, USA), which carries the CMV promoter and the SV40 polyadenylation signal supporting a ubiquitous expression, was chosen for this experiment. The B6D2F2 mouse embryos were collected and treated for 5 seconds in Acidic Tyrode's solution (AT) (P/N T1788, Sigma Aldrich), washed twice in KOSMaa/BSA (P/N Zeks-050, Zenith Biotech), and placed into 25 μL of Opti-MEM (P/N 31985, Life Technologies). The embryos in 25 μL were then mixed with an equal volume (25 μL) of pMAXGFP at 80 ng/μL in TE buffer (10 mM Tris, 0.1 mM EDTA, pH 7.5) to arrive at a final DNA concentration of 40 ng/μL, and the mixture was loaded into a 1-mm electroporation cuvette, and electroporated using the settings of: 30 volts, pulse duration 1 ms, two pulses, pulse interval 100 ms (ECM830, BTX).

After electroporation, the embryos were recovered with 100 μL of pre-equilibrated KOSMaa/BSA, and were subsequently cultured for 3.5 days in a tissue culture incubator (37° C., 5% CO₂). After 3.5 days in culture, the embryos were observed under a fluorescent microscope for GFP expression. Fluorescence was not observed among the 3 embryos examined (Experiment 1).

Since the zona pellucida layer may present a physical barrier for plasmid entry into the zygotes, in Experiments 2 and 3, Applicant sought to weaken or break the zona pellucida layer by poking a hole on the zona, by removing the zona, or by treating the embryos in AT for 10 seconds, while at the same time varying the concentrations of pMAXGFP from 20 ng/μL, to 40 ng/μL, to 100 ng/μL. Again, no fluorescence among the embryos examined was observed.

Other reporter systems, including an oligonucleotide labeled with 6-fluorescein amidite (6-FAM) (40, 100, and 500 ng/μL), Turbo GFP mRNA (40 ng/μL), eGFP mRNA (25 ng/μL) or mCherry mRNA (40 and 100 ng/μL) and did not observe fluorescence among the embryos examined (Experiments 4, 5, and 6). Only one embryo with GFP signal was observed in one case, but the embryo was dead or developmentally retarded.

### Example 2 Electroporation of Mouse Zygotes and Presumptive Zygotes with CRISPR/Cas System

This experiment demonstrates the delivery of CRISPR/Cas system, using the methods of the invention, to mouse zygotes or presumptive zygotes (“zygotes” for short) for CRISPR/Cas-mediated targeted gene disruption.

For this purpose, Tet1 exon 4 and Tet2 exon 3 were chosen as targets, using the guide RNAs described previously (Wang et al., 2013, incorporated herein by reference). The convenience of the Tet1 and Tet2 systems is that the Sac1 (Tet1) or the EcoRV (Tet2) restriction sites overlap the PAM (protospacer adjacent motif) proximal sequences. As such, Restriction Fragment Length Polymorphism (RFLP) analysis can be used to detect mutant alleles, using PCR products amplified from embryos that encompass the target sites (Wang et al., 2013).

In Experiment 6, mouse B6D2F2 zygotes were first treated with AT for 10 seconds, mixed with Cas9 mRNA/Tet1 sgRNA at 40/20 ng/μL or 100/50 ng/μL, and electroporated as described. The embryos were then cultured for 3.5 days until they developed into blastocysts. The embryos were then harvested for whole genome amplification and PCR analysis.

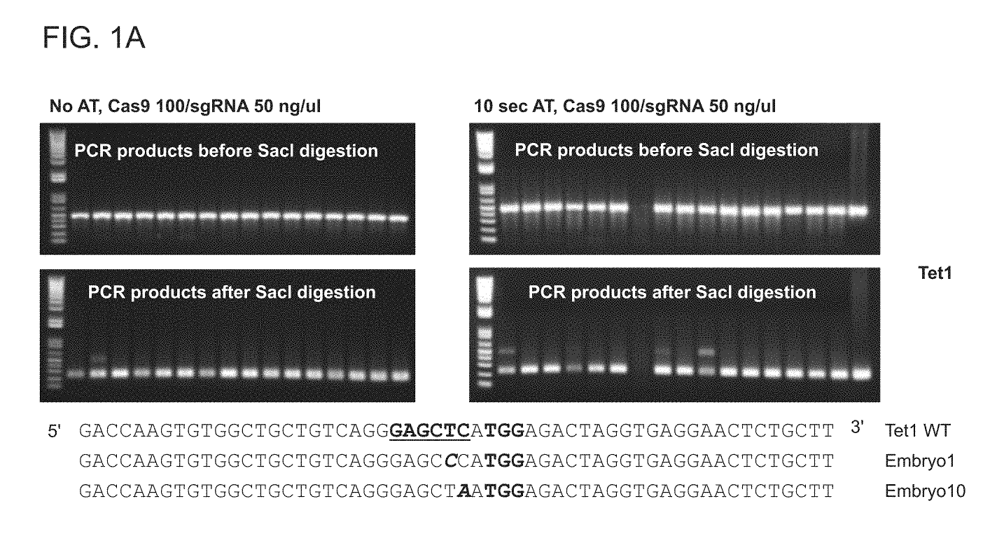

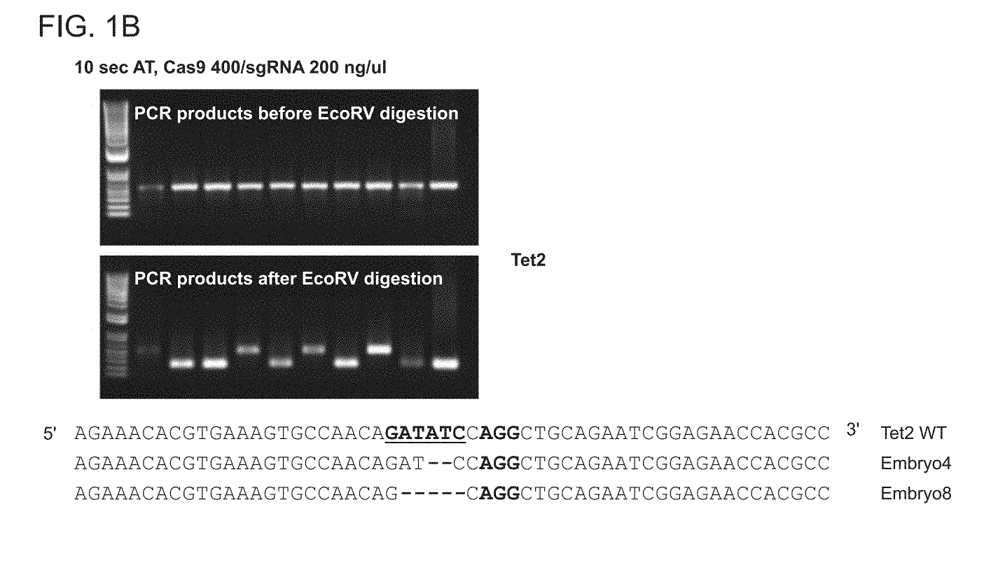

PCR products encompassing the target site were analyzed by RFLP using the Sac1 restriction enzyme. When the mixture of Cas9 mRNA (40 ng/μL) and Tet1 sgRNA (20 ng/μL) was used, no mutation was detected among the embryos developed from the electroporated zygotes. When the mixture of Cas9 mRNA (100 ng/μL) and Tet1 sgRNA (50 ng/μL) was used, 3 out of 16 AT-treated embryos and 1 out of 16 embryos without AT treatment were found to carry mutant alleles at the Tet1 locus, based on RFLP analysis (FIG. 1A). The mutations were further confirmed by Sanger sequencing (FIG. 1A).

Next, in Experiment 7, an even higher concentration of Cas9 mRNA (400 ng/μL)+Tet2 sgRNA (200 ng/μL) were electroporated into mouse zygotes, and 4 out of 9 AT-treated embryos were found to carry biallelic mutations at the Tet2 locus (FIG. 1B).

Therefore, both Tet1 and Tet2 genes can be efficiently targeted in mouse zygotes using electroporation-delivered CRISPR/Cas system (e.g., Cas9 mRNA+sgRNA).

Meanwhile, electroporation of CRISPR/Cas-encoding plasmids (as an alternative to the delivery of CRISPR reagents in the form of Cas mRNA and guide RNA) was attempted using different plasmid concentrations, namely 200 ng/μL, 400 ng/μL, and 800 ng/μL plasmid DNA. In the 200 ng/μL group, one mutant embryo out of 12 total screened was identified. Therefore, electroporation of DNA into zygotes can also be achieved, although in the case of CRISPR/Cas system, the mRNA/guide RNA combination appeared to be more efficient than DNA.

In this experiment and the other experiments using the CRISPR/Cas system, Cas9 mRNA and sgRNA were produced using the following procedure.

Briefly, the px330 plasmid carrying the wild type Cas9 gene (Cong et al., 2013) served as the DNA template for amplification of the Cas9 coding sequence in a polymerase chain reaction (PCR). The T7 promoter sequence was added to the forward primer and paired with the reverse primer flanking the coding sequence of the Cas9 wild type gene. PCR product was purified using the Qiagen column (Qiagen), and in vitro transcription (IVT) was performed using the mMESSAGE mMACHINE T7 ULTRA Transcription kit (Life Technologies). For sgRNA synthesis, the T7 promoter sequence was added to sgRNA template/forward primer and the IVT template was generated by PCR amplification. The T7-sgRNA PCR product was column purified and used as the template for IVT using MEGAshortscript T7 kit (Life Technologies). Both the Cas9 mRNA and the sgRNAs were purified using MEGAclear kit (Life Technologies) and eluted in elution buffer provided with the kit. Aliquots from an IVT reaction were separated on agarose gel to assess quality from a reaction.

Surveyor assay was performed as described (Guschin et al., 2010). Genomic DNA from mice or embryos was extracted and PCR performed using gene-specific primers under the following conditions: 95° C. for 5 min; 35×(95° C. for 30 s, 60° C. for 30 s, 68° C. for 40 s); 68° C. for 2 min; hold at 4° C. PCR products were then denatured, annealed, treated with Surveyor nuclease (Transgenomic). The products were then separated on a 10% acrylamide Criterion TBE gel (BioRad), stained with ethidium bromide and visualized. For RFLP analysis, 10 μl of Tet1 or Tet2 PCR products were digested with Sac (Tet1) or EcoRV (Tet2) and separated on a GelRed (Biotium)-stained agarose gel (2%). For sequencing, PCR products were sequenced directly or cloned into the Topo Cloning Kit (Invitrogen), and individual clones sequenced by Sanger sequencing.

### Example 3 Development of Electroporated Mouse Zygotes

In vitro culture and analysis of zygotes is an important approach, based on which a large number of parameters related to gene editing technologies can be tested and analyzed, with a quick turnaround time and preferably a lower operating cost. However, when conventional in vitro culture system was used to culture electroporated zygotes, subsequent embryonic development seemed to be retarded or aborted. Often, after electroporation of Cas9 mRNA/sgRNA at varied concentration ranges of, for example, 50/25 ng/μL to 1000/500 ng/μL, embryos progressed to different stages of development at the end of the 3.5-day culture period, including those of 1-cell, 2-cell, or 4-cell stages, morula stage, and occasionally blastocyst stage (Experiment 8). In contrast, control embryos that had not been electroporated reached the blastocyst stage at the end of the same time period, whether the control embryos had been pre-treated with AT or not. In some experiments (Experiments 10 and 11), embryos among all the groups in an experiment failed to reach blastocyst stage.

Such initial failures in culturing electroporated zygotes appear to suggest that zygotes may not survive the electroporation process and continue to develop into live-born animal, regardless of whether the desired genetic modification occurs in the electroporated zygotes. The realization that AT treatment could damage the zygotes excessively to retard embryonic development, and that the reagents delivered, such as the Cas9 mRNA and the guide RNA, could be toxic to the zygotes and their subsequent development, further casts doubts as to using electroporation as a viable means to permanently introduce germline transmittable genetic modifications in the animal.

In attempting to resolve the poor in vitro development issue of the electroporated zygotes, Applicant surprisingly identified a solution by changing the in vitro culturing condition (Table 1), more specifically, by washing away any potentially harmful substance in the electroporation medium, which typically comprise a polynucleotide buffer (e.g., TE buffer) and reduced-serum medium (e.g., OPTI-MEM® medium). The dramatic effects of this, on survival and development of the electroporated zygotes, are demonstrated in the experiments described here.

In particular, B6D2F1/J donor female mice were superovulated to maximize embryo yield. Each donor female received 5 IU (i.p. injection) of Pregnant Mare Serum Gonadotrophin (PMSG), and, 47 hours later, followed by 5 IU (i.p. injection) of human chorionic gonadotrophin (hCG).

Immediately post administration of hCG, the female was mated 1:1 with a B6D2F1/J stud male. About 24 hours later, the female was checked for the presence of a copulation plug. Females displaying a copulation plug were euthanized and their oviducts were excised and placed into M2 media. The oviducts were then placed in M2 media with hyaluronidase added at a concentration of 0.3 mg/mL. The oocyte clutch was removed by lysing the ampulla and allowing to incubate in the M2 medium containing hyaluronidase, until the cumulus cells fell off. Zygotes were collected and passed through two washes of fresh M2 media before being placed in microdrops of RCVL media that had been equilibrated under oil for 24 hours in a COOK MINC benchtop incubator.

Acidic Tyrode's solution was thawed, and 100 μL drops were placed in a petri dish, with 1 drop for every 50 zygotes to be treated. Zygotes identified for EP (electroporation) were removed from the RCVL media, and placed in a (pre-warmed) 100 μL drop of M2 media. In groups of 50, the zygotes were placed in the Acidic Tyrode's solution for 10 seconds, and then removed and washed through three 100 μL drops of pre-warmed M2 media. The treated zygotes were then placed into 5-10 μL drops of OPTI-MEM® medium in preparation for electroporation. The number of zygotes per OPTI-MEM® medium drop and the number of drops varied with the parameters of the experiment being performed.

For both Experiments 14 and 15, CRISPR mixture contains Cas9 mRNA/Tet2 sgRNA at 200/100, 400/200, and 600/300 ng/μL were used. In addition, for Experiment 15, a donor oligonucleotide carrying mutation for converting the EcoRV site (GATATC) into a EcoRI site (GAATTC) was also provided at 200, 400, or 200 ng/μL. The mixture was brought up to μL per experiment group in TE buffer (10 mM Tris, 0.1 mM EDTA, pH7.5) and added to embryos in 10 μL OPTI-MEM®. The total content of 20 μL was then loaded into a 1-mm cuvette and electroporation delivered (30 volts, pulse duration 1 ms, 2 pulses, pulse interval 100-1000 ms).

Upon completion of electroporation, if it is desirable to wash the electroporated gametes or preimplantation embryos prior to the next steps (e.g., in vitro culturing and/or transferring to a pseudopregnant female), the embryos were recovered from the cuvette in 100 μL of pre-warmed KSOMaa/BSA media and serially passed through three 100 μL drops of pre-warmed M2 media to wash off any remaining electroporation solution (1:1 of TE buffer and OPTI-MEM® medium). The zygotes were then assessed for viability. Those judged to be healthy were transferred into a cryovial containing 950 μL of pre-warmed M2 media for transport to the specific pathogen free (SPF) surgical suite.

In the SPF facility, the zygotes were removed from the cryovial using a p1000 pipette and placed into culture drops of RCVL media that had been equilibrated under oil for 24 hours in a COOK MINC benchtop incubator. At the time of transfer, the embryos were removed from the RCVL microdrop and placed in a pre-warmed 100 μL drop of M2 media and transferred into a CBYB6F1/J pseudo pregnant female in accordance to standard protocol, such as Jackson Laboratories LAH (laboratory animal health) routine procedure 94-09.

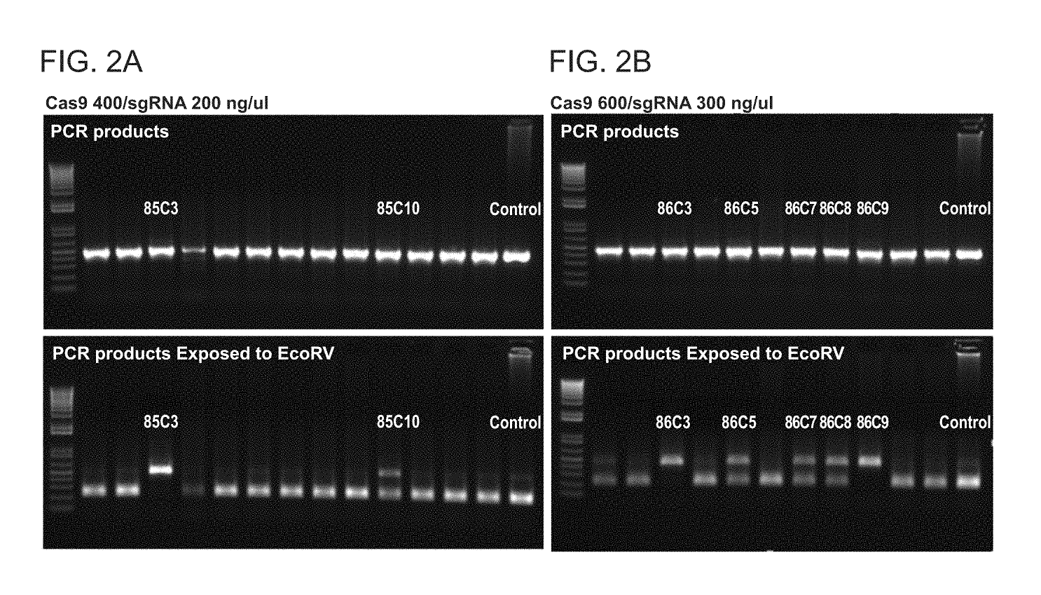

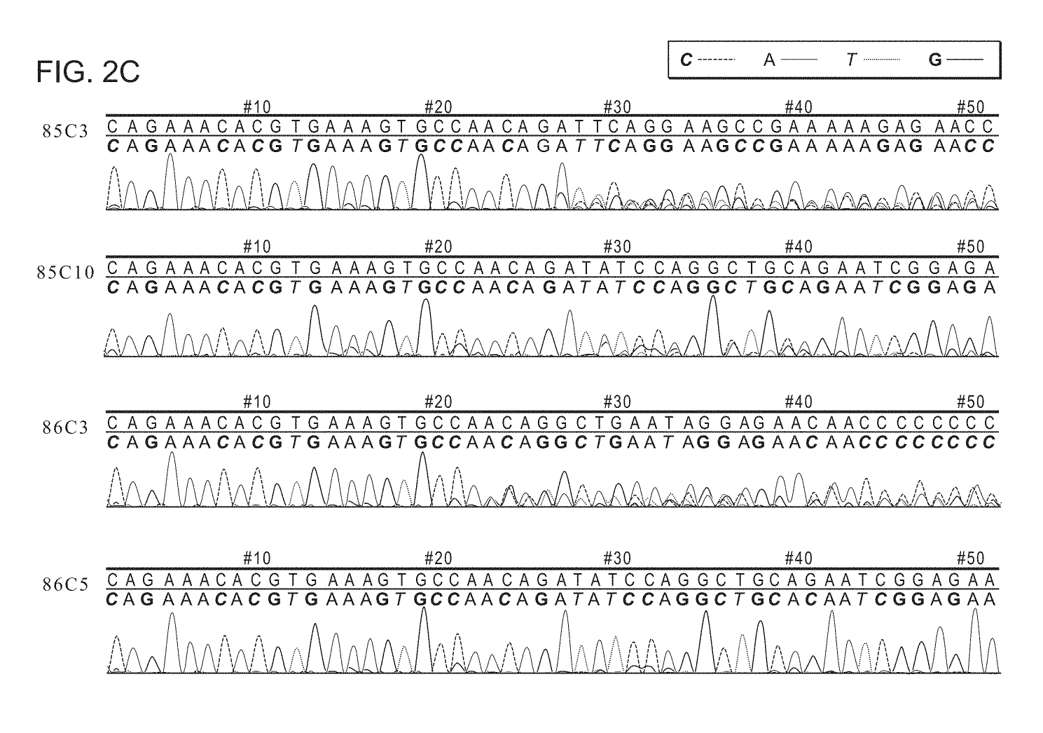

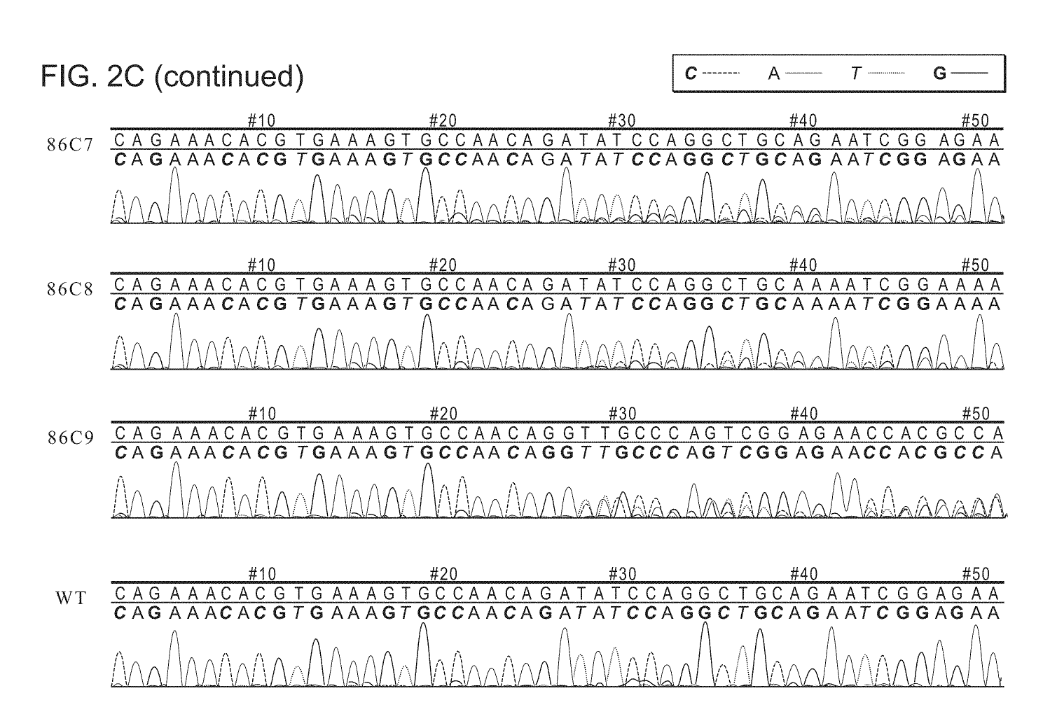

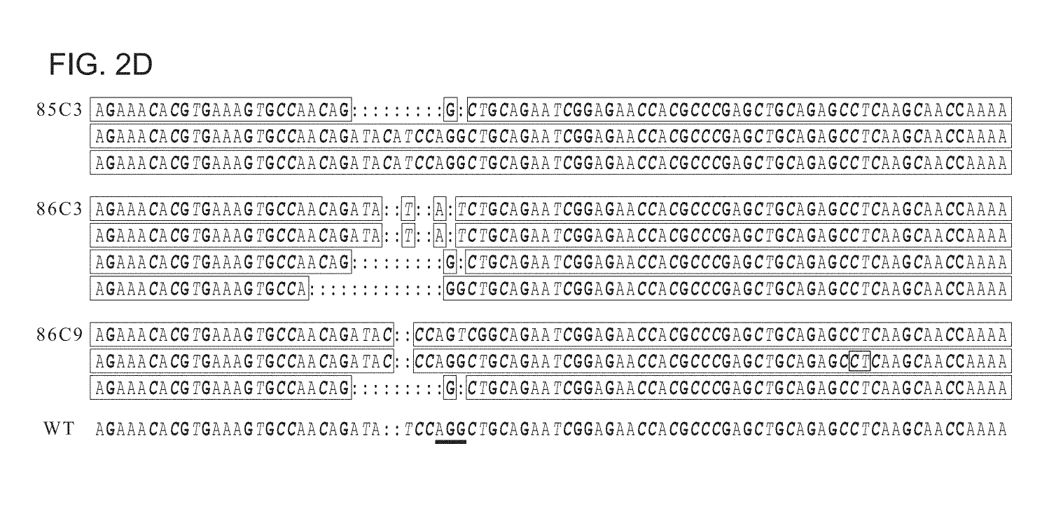

A subset of the mice was analyzed upon births. For these, tail biopsy samples were taken when the mice were born and analyzed by RFLP with EcoRV digestion (FIGS. 2A and 2B). PCR product from two mice out of 13 screened from the group of Cas9 mRNA 400 ng/μL and sgRNA 200 ng/μL was resistant (founder 85C3) or partially resistant to (85C10) to EcoRV digestion (FIG. 2, Panel A).

When Cas9 mRNA and sgRNA were used a higher concentration of 600 ng/μL and 300 ng/μL, respectively, five mice were positive as analyzed by the RFLP assay (founders 86C3, 86C5, 86C7, 86C8 and 86C9), among which two (86C3 and 86C9) were completely resistant while 3 (86C5, 86C7 and 86C8) were partially resistant to EcoRV digestion. Sanger sequencing of the PCR products confirmed presence of the mutant alleles for 6 out of the 7 mice (FIGS. 2C, 85C3, 85C10, 86C3, 86C7, 86C8 and 86C9), while one may carry mutant alleles of low abundance (86C5). Furthermore, when the PCR products from founders 85C3, 86C3 and 86C9 were cloned into the pCR2.1-Topo vector and individual clones sequenced, INDEL mutations were detected, as expected, precisely located at the PAM proximal region of the Tet2 target site (FIG. 2D).

Remaining mice from these two experiments were analyzed when they were one week old. Tail biopsy samples were taken and analyzed as previously described by RFLP, and the results confirmed by Sanger sequencing of PCR products amplified from these mice. As summarized in Table 1, it was observed a concentration dependent improvement in founder efficiency between these two experiments. When Cas9 mRNA was used at 200 ng/μL and sgRNA 100 ng/μL, 1 founder was identified among the 34 screened (3.2%). When the concentration was increased to Cas9 mRNA 400 ng/μLand sgRNA 200 ng/μL, founder efficiency increased to 48.3% (14 founder mice among the 29 screened). When the concentration was further increased to Cas9 mRNA 600 ng/μL and sgRNA 300 ng/μL, efficiency was 45.5% (15 founder mice among the 33 screened). Remarkably, for Experiment 15, 100% founder efficiency was achieved, with all 11 mice screened found to carry mutant alleles (FIG. 3).

CRISPR-mediated HDR was also tested in Experiment 15, in which donor oligonucleotide was incorporated in the experimental design.

Specifically, zygotes were electroporated and mice were born and genotyped. A PCR product of 466 bps was amplified, which amplification product encompasses the target site, exposed to EcoRV digestion to detect alleles carrying NHEJ mutations and to EcoRI digestion to detect the HDR allele. Although successful HDR alleles were not detected among other experimental groups, two founder mice carrying alleles that were cleaved by EcoRI (FIG. 3, Panel A, 86EP11 and 86EP14) were detected, and they were obtained from the group that was electroporated with Cas9 mRNA at 400 ng/μL, sgRNA targeting the 3′ UTR of the Tet2 locus at 200 ng/μL, and ssDNA donor at 400 ng/μL. Among this group, all 11 mice carry alleles that are resistant to EcoRV digestion. Founders 86EP11 to 86EP15 do not seem to carry a detectable level of alleles that could be cleaved by EcoRV, suggesting presence of NHEJ mutations from all 11 founder mice. When the 11 samples were exposed to EcoRI, some of the alleles from samples 86EP11 and 86EP14 were shown to be partially digested by EcoRI, suggesting presence of the HDR alleles from these two samples.

PCR products were then cloned from founders 86EP11 and 86EP14 into the pCR2.1-TOPO vector, and individual clones were sequenced. As shown in FIG. 3, Panel B, sample 86EP11-4 and 86EP14-4 carry the EcoRI sites, suggesting successful incorporation of this site from donor ssDNA mediated by CRISPR/Cas.

A variety of different electroporation set up parameters were further tested in a series of experiments, and their results were summarized below:

1. 20V, 2 pulses, 100 ms interval, 0 washes
a. Cas9 mRNA 200 ng/uL+Tet2 sgRNA 100 ng/μL
b. Results: 12% reached blastocyst stage (2/17), no gene targeting observed

2. 20V, 2 pulses, 100 ms interval, 3 washes
a. Cas9/Tet2: 500/250, 400/200, 200/100 ng/μL
b. Results: 91% blastocyst on average (50/55)

3. 20V, 2 pulses, 1 s interval, 3 washes
a. Cas9/Tet2: 800, 600, 400, 300, 200, 100 ng/μL
b. Results (batch 1) at 200 ng/μL: 100% blast (16/16), 14.3% targeting (1/7)
c. Results (batch 2) at 200, 300, 400 ng/μL: 95.8% blasts (46/48), results pending
d. Results (batch 3): multiple washes, embryos transferred to pseudopregnant female mice, births pending

4. 20V, 3 pulses, 1 s interval, 1 wash
a. Cas9/Tet2: 400, 200 ng/μL
b. Results (batch 1) at 200 ng/μL: 82.3% blasts (14/17), no targeting
c. Results (batch 2) at 400 and 200 ng/μL: 93.8% blasts (30/32), results pending

5. 20V, 3 pulses, 1 s interval, 3 washes
a. Cas9/Tet2: 200 ng/μL
b. Results: 88.9% blasts (16/18), no targeting

6. 30V, 2 pulses, 1 s interval, 3 washes
a. Cas9/Tet2: 400, 300, 200 ng/μL
b. Results at 200 ng/μL: 94% blast (15/18), no targeting
c. Results at 300 ng/μL: 84% blast (16/19), 16% targeting (3/19), no targeting at 400 ng/μL

In summary, the results suggest that successful genetic modification using electroporated CRISPR/Cas system depends on the concentration of the CRISPR/Cas reagents used. In an electroporation experiment, as compared to the traditional microinjection experiments, reagent concentration stops being a limiting factor, and it was found that the use of higher concentration reagents (e.g., above 400 ng/μL for Cas9 mRNA and 200 ng/μL for sgRNA) generates gene edited mice.

The data also suggests that weakening Zona Pellucida, e.g., by AT treatment, may be beneficial for zygote electroporation. However, prolonged treatment should be avoided.

Lastly but not the least, CRISPR-mediated gene targeting using the subject methods could be successfully conducted and with certain variations in parameters in experimental setup. To ensure successful development of the electroporated zygotes, it may be required to rinse once or multiple times (e.g., 2, 3, 4 times) the electroporated zygotes with a physiological solution or media optimal for embryo development, upon completion of electroporation. For example, the electroporated zygotes can be serially passed through (100 μL) drops of pre-warmed M2 media to wash off any remaining electroporation solution (e.g., 1:1 of TE buffer and OPTI-MEM®) before the zygotes are placed into culture or transferred to the uterine environment.

TABLE 1
Results Summary for Experiments 14 and 15

| | | | BTX ECM830 Condition # Embryos | | | | | | | | NHEJ | |
|---|---|---|---|---|---|---|---|---|---|---|---|---|
| | | | | | Pulse | Pulse | | | | | Mutant/ | |
| | Well | | Voltage | # | Interval | Duration | at | post | | Conc. | Total | Percentage |
| Experiment | ID | AT (s) | (v) | Pulses | (ms) | (ms) | EP | EP | CRISPR Reagents | (ng/ul) | Mice | (%) |
| 14 | A1 | 10 | 27 | 2 | 125 | 1.5 | 20 | 17 | Cas9/Tet2 sgRNA | 200/100 | 0/11 | 0 |
| | B1 | 10 | 27 | 2 | 128 | 1.5 | 20 | 18 | Cas9/Tet2 sgRNA | 400/200 | 1/10 | 10 |
| | C1 | 10 | 27 | 2 | 128 | 1.5 | 20 | 18 | Cas9/Tet2 sgRNA | 600/300 | 4/6 | 67 |
| | D1 | 10 | 27 | 2 | 125 | 1.5 | 37 | 35 | Cas9/Tet2 sgRNA | 200/100 | 1/10 | 10 |
| | E1 | 10 | 27 | 2 | 128 | 1.5 | 37 | 34 | Cas9/Tet2 sgRNA | 400/200 | 2/8 | 29 |
| | F1 | 10 | 27 | 2 | 128 | 1.5 | 37 | 26 | Cas9/Tet2 sgRNA | 600/300 | 4/8 | 50 |
| | G1 | 10 | 27 | 2 | 128 | 1.5 | 20 | 17 | control | | 0/8 | 0 |
| | H1 | no AT | no EP | | | | 12 | 12 | control | | 0/3 | 0 |
| 15 | A1 | 10 | 27 | 2 | 128 | 1.5 | 20 | 18 | Cas9/Tet2 | 200/100/200 | 0/10 | 0 |
| | | | | | | | | | sgRNA/donor | | | |
| | B1 | 10 | 27 | 2 | 130 | 1.5 | 20 | 20 | Cas9/Tet2 | 400/200/400 | 11/11 | 100 |
| | | | | | | | | | sgRNA/donor | | | |
| | C1 | 10 | 27 | 2 | 130 | 1.5 | 20 | 19 | Cas9/Tet2 | 600/300/200 | 4/11 | 36 |
| | | | | | | | | | sgRNA/donor | | | |
| | D1 | 10 | 27 | 2 | 128 | 1.5 | 40 | 36 | Cas9/Tet2 | 200/100/200 | 0/3 | 0 |
| | | | | | | | | | sgRNA/donor | | | |
| | E1 | 10 | 27 | 2 | 130 | 1.5 | 40 | 35 | Cas9/Tet2 | 400/200/400 | (no live | |
| | | | | | | | | | sgRNA/donor | | births) | |
| | F1 | 10 | 27 | 2 | 128 | 1.5 | 40 | 32 | Cas9/Tet2 | 600/300/200 | 3/8 | 38 |
| | | | | | | | | | sgRNA/donor | | | |
| | G1 | 10 | 27 | 2 | 130 | 1.5 | 20 | 19 | control | | 0/12 | 0 |
| | H | no AT | no EP | | | | 14 | 14 | control | | 0/10 | 0 |

A detailed illustrative (but non-limiting) embodiment of the method of the invention is provided herein below:
1. Aliquots of 100 μL KSOMaa Evolve (P/N Zeks-050, Zenith Biotech), supplemented with BSA (P/N A2153, Sigma-Aldrich) at 1 mg/mL, were deposited onto a 96-well flat bottom tissue culture plate and equilibrated in HeraCell incubator at 37° C./5% CO₂, prior to receipt of one cell zygotes.
2. B6D2F1 female mice were injected intraperitoneally (i.p.) with 5 international units (IU) of pregnant mare's serum gonadotropin (PMSG, P/N HOR-272, ProSpec, Rehovot, Israel), and were followed 48 hours later with an i.p. injection of human chorionic gonadotropin (hCG, P/N HOR-250, ProSpec, Rehovot, Israel). The female mice were then mated with B6D2F1 male mice. Those that were positive for a copulation plug were euthanized by cervical dislocation (CD). Fertilized embryos were flushed from the excised reproductive tracts.
3. One cell zygotes from B6D2F2 mice were collected, treated for 10 seconds in Acidic Tyrode's Solution (e.g., Sigma-Aldrich, Cat. No. T1788; or equivalent such as EMBRYOMAX® P/N MR-004-D, EMD Millipore), washed twice with KSOMaa Evolve, and placed in 10 μL drops of prewarmed OPTI-MEM® medium (P/N 31985, Gibco) in a tissue culture dish or plate (35 mm, P60, 96 well).
4. “Biological material” (for CRISPR, it consists essentially of Cas9 mRNA, sgRNA, and optionally donor oligonucleotide; or DNA encoding the same as an alternative) was reconstituted into a 10 μL volume with 10 mM Tris, pH 7.5, and 0.1 mM EDTA, and added to the 10 μL drop of zygotes in OPTI-MEM® medium.
5. Zygotes immersed in the “biological material” were removed and deposited into the chamber of the 2-mm cuvette for BTX Model 610 (P/N 450124, Harvard Apparatus). Tap the cuvette to ensure polynucleotide/zygote solution or suspension move to between the two conductors of the cuvette, with no air bubble trapped in the mixture.
6. Place cuvette into the BTX cuvette chamber (ECM 830) and electroporate using the following settings (or variations thereof, such as those disclosed herein):
   a. Voltage: 30V
   b. Pulse Interval: 125-130 ms
   c. Pulse Duration: 1 ms
   d. # Pulses: 2
7. Using supplied plastic pipette from cuvette package, remove 80-100 μL of prewarmed media (e.g., KSOMaa/BSA media) from respective well of a 96-well plate and gently add the media to the cuvette. Remove all contents from the cuvette and expel the mixture to the well.
8. Serially passing the electroporated zygotes through two-three 100 μL drops of pre-warmed M2 media to wash off any remaining electroporation solution.
9. Immediately place the 96-well plate carrying the washed zygotes back into the incubator.
10. Embryos were cultured at 37° C./5% CO₂ for 3.5 days to reach blastocyst stage of development and then harvested for analysis.
11. Genomes of the embryos were amplified using the GENOMEPLEX® Single Cell Whole Genome Amplification Kit (P/N GA4, Sigma-Aldrich). Amplified genome was then used as template in a PCR reaction (ACCUPRIME™ Taq DNA Polymerase System, P/N 12339-016, Life Technologies; or PrimeStar GXL, P/N R050B, Clontech) with a primer pair encompassing the target site. PCR products were cleaned and nucleotide sequences analyzed by Sanger sequencing.
12. Cas9 mRNA, sgRNA and the optional DNA donor were synthesized, and injection mixture prepared as described previously (Wang et al., 2013; Yang et al., 2013).

### Example 4 Improved Multi-Cycle Electroporation of Mouse Zygotes

The methods described in the examples above occasionally exhibit variations for different target genes. For example, in some trials at the Aicda and Rosa26 loci, non-homologous end joining (NHEJ) occurred at relatively low efficiency (˜0% and 17%, respectively) (Data not shown). In another parallel comparative study between microinjection and electroporation, methods based on the examples above appeared not able to generate large segment deletion in the Smc1b locus, while the microinjection had high efficiency (data not shown). It is not completely clear whether such observations were due to locus-specific effects.

However, using these genomic loci as targets, this example demonstrates that increased genome editing efficiency can be achieved using the improved methods described herein. In addition, the improved methods can also introduce different genetic modifications, including precise nucleotide changes, large segment deletion, and targeted short segment insertion, into a target locus.

One improvement is to use a series (e.g., multiple cycles) of electroporations instead of just one round/cycle of electroporation as described above. To determine the optimal series of electroporation cycles, 1, 4, 6, and 8 series of electroporation cycles were used, with the same or similar setting as used before (e.g., 30V; 2 pulses; 1 or 1.5 ms pulse duration; 100 ms interval), and the observed survival rates of the electroporated embryos in two cell and blastocyst stages were compared.

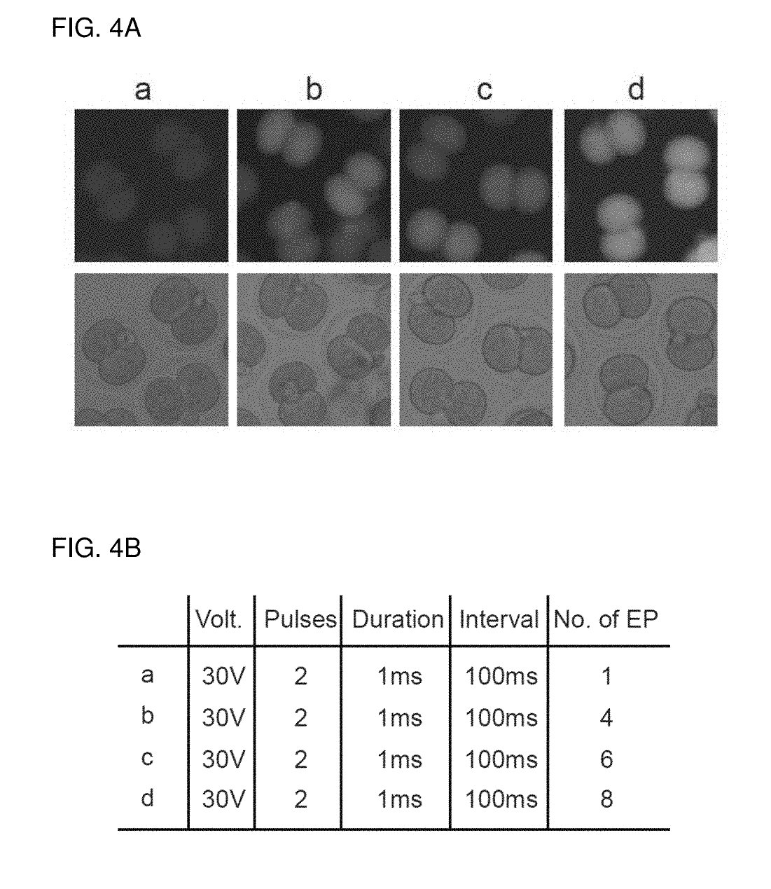

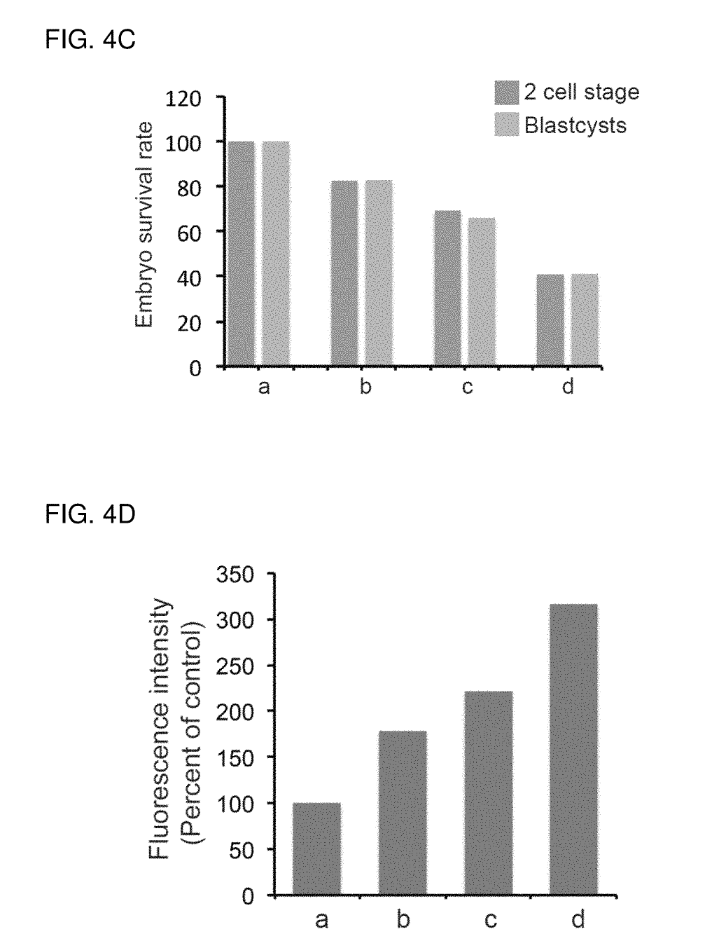

While survival rate reduced along with increased times of electroporation, six cycles of electroporation resulted in a reasonable rate of in vitro development of blastocysts (FIG. 4C). Importantly, the delivery efficiency of a marker—GFP mRNA—increased significantly when more cycles of electroporation was applied (FIG. 4D).

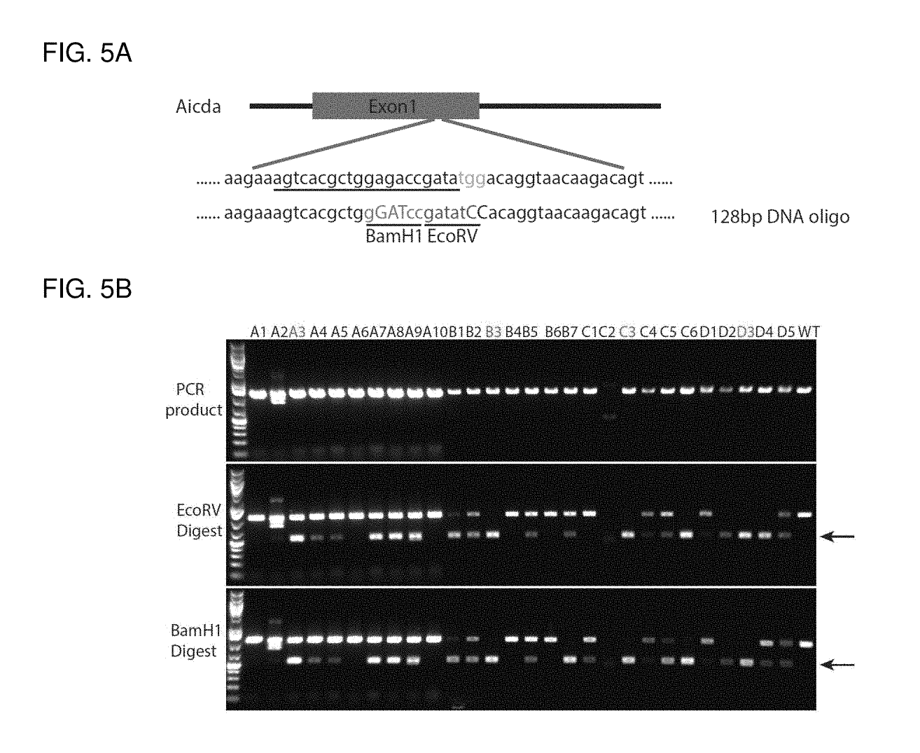

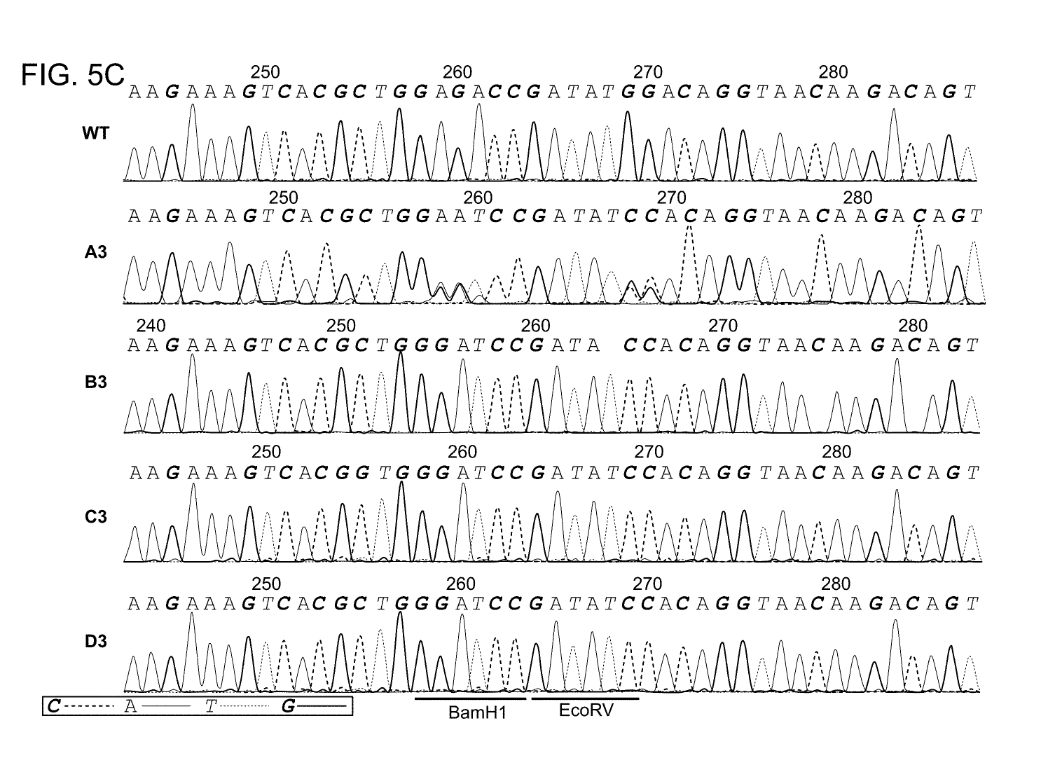

Next, Aicda were used as the target to test whether applying multiple cycles of electroporation improves targeting efficiency. B6D2F2/J embryos were prepared through flushing the fresh oviduct. The fertilized embryos were first treated with AT (acidic tyrode) for 10 seconds, and then washed extensively before electroporation. Mouse zygotes were loaded into 10 μL of Opti-MEM, and then mixed with 10 μL of Cas9 mRNA (600 ng/μL), sgRNA targeting Aicda (300 ng/μL), DNA oligo (600 ng/μL). The DNA oligo contains DNA sequence flanking the target region and a few nucleotide changes converting the original target sequence (gagaccgatatgg) to the sequence including a BamH1 site (GGATCC) and an EcoRV site (GATATC) (FIG. 5A). Zygotes were then electroporated in a cuvette at the same setting as described above for 1, 4, 6 or 8 series/cycles of electroporation (FIG. 4B). After electroporation, the embryos were immediately transferred into pseudopregnant mice. Two weeks after the pups were born, tissue samples were collected for genotyping.

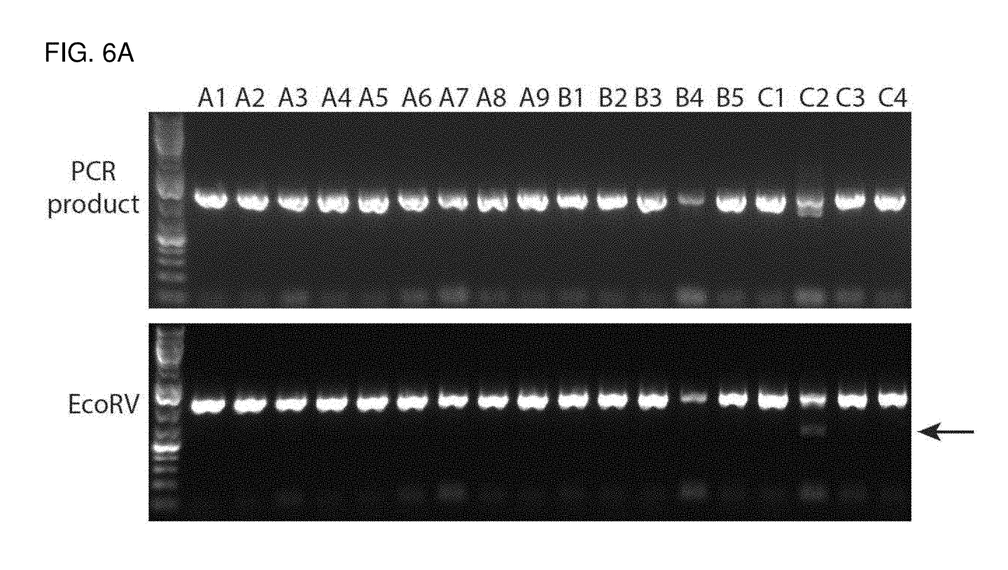

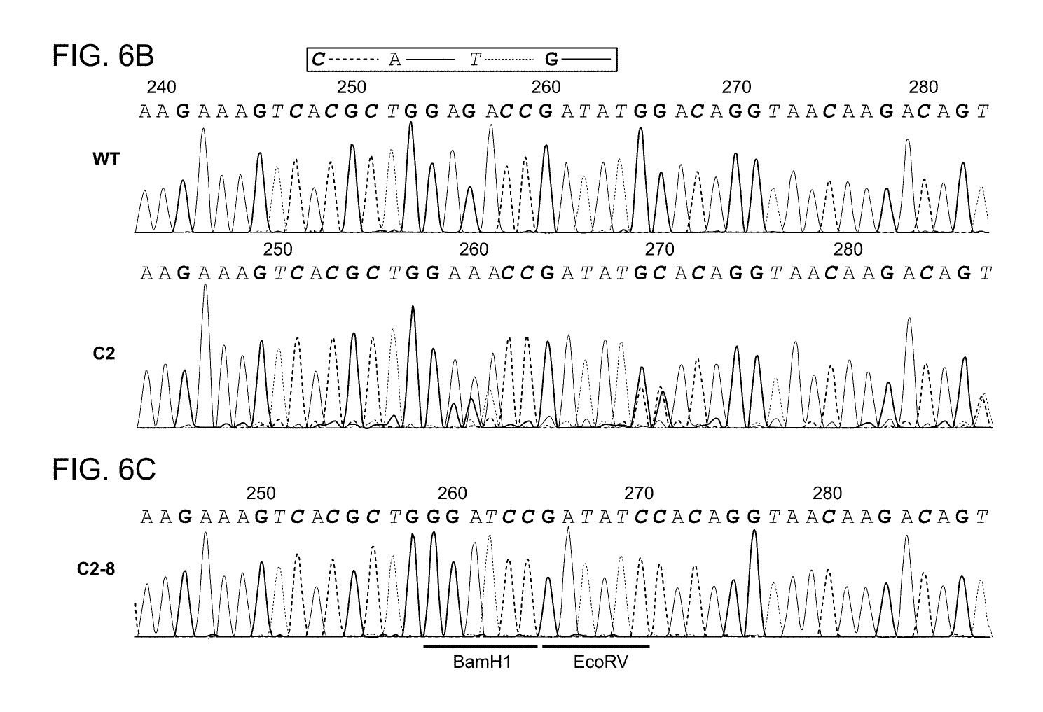

The PCR products encompassing the target site were digested with EcoRV and BamHI independently, and sequenced. As shown in FIGS. 6A-6C and the table immediately below, there was 1 out of 4 mice with KI (knock-in) after 6 times/cycles of electroporation. Meanwhile, all other groups did not have KI.

Using Cas9 mRNA (Homemade) to Generate
Aicda genome Knock-In (KI)

| Cycles of | Embryos | | Total mice | Percentage |
|---|---|---|---|---|
| Electroporation | transferred | Mice born | analyzed | of NHEJ/KI |
| 1 | 15 | 7 | 7 | 0/0 |
| 4 | 15 | 7 | 7 | 0/0 |
| 6 | 15 | 4 | 4 | 0%/25% |
| 8 | 15 | 0 | 0 | n.a. |

Commercial Cas9 mRNA was then used for electroporation with the same setting. Better live-born rate was observed upon multiple cycles of electroporation. As shown in the table immediately below, although there were no successful KI for most of the groups, there were 3 out of 11 mice that had KI after 8 series of electroporation.

Using Cas9 mRNA (Commercial) to Generate
Aicda genome Knock-In (KI)

| Cycles of | Embryos | | Total mice | Percentage |
|---|---|---|---|---|
| Electroporation | transferred | Mice born | analyzed | of NHEJ/KI |
| 1 | 15 | 7 | 7 | 0/0 |
| 4 | 15 | 10 | 10 | 0/0 |
| 6 | 15 | 9 | 9 | 0/0 |
| 8 | 15 | 11 | 11 | 0%/27.27% |

These data suggest that multiple times/cycles of electroporation can increase the efficiency, although the efficiency is still lower than that of microinjection for certain genomic loci.

To further improve the efficiency, Cas9 protein was delivered together with other CRISPR components, at the same setting as used for Cas9 mRNA. In some experiments, Cas9 protein at 250 ng/μL was used for all the experimental groups. Certain electroporated embryos were immediately transferred into pseudopregnant mice after electroporation, and live mice were derived. Certain other electroporated embryos were cultured to blastocyst stage and were harvested for genotyping using RFLP (restriction fragment length polymorphism) and DNA sequencing.

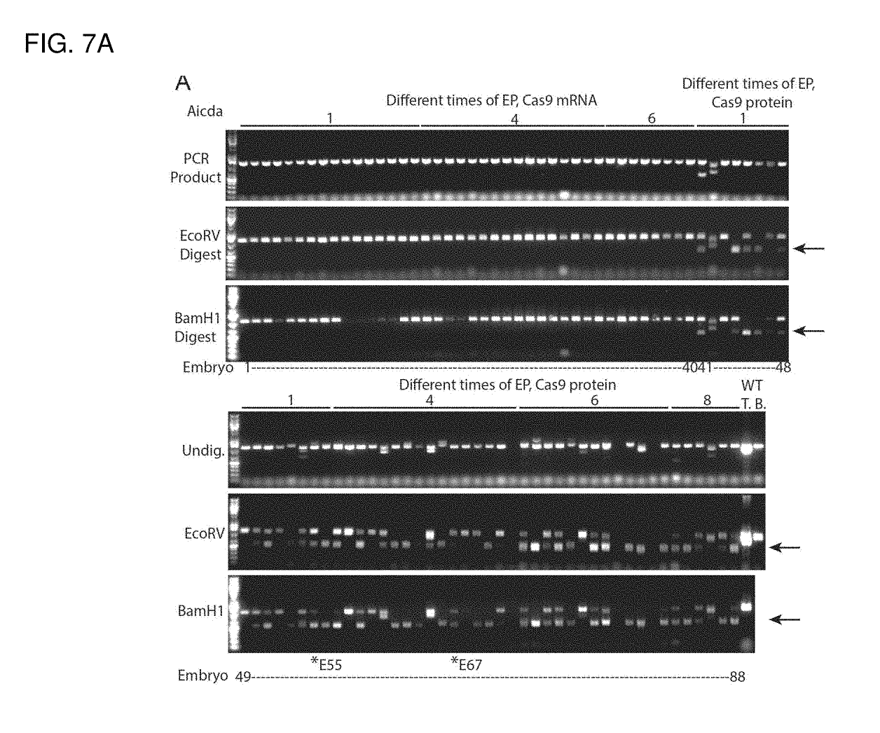

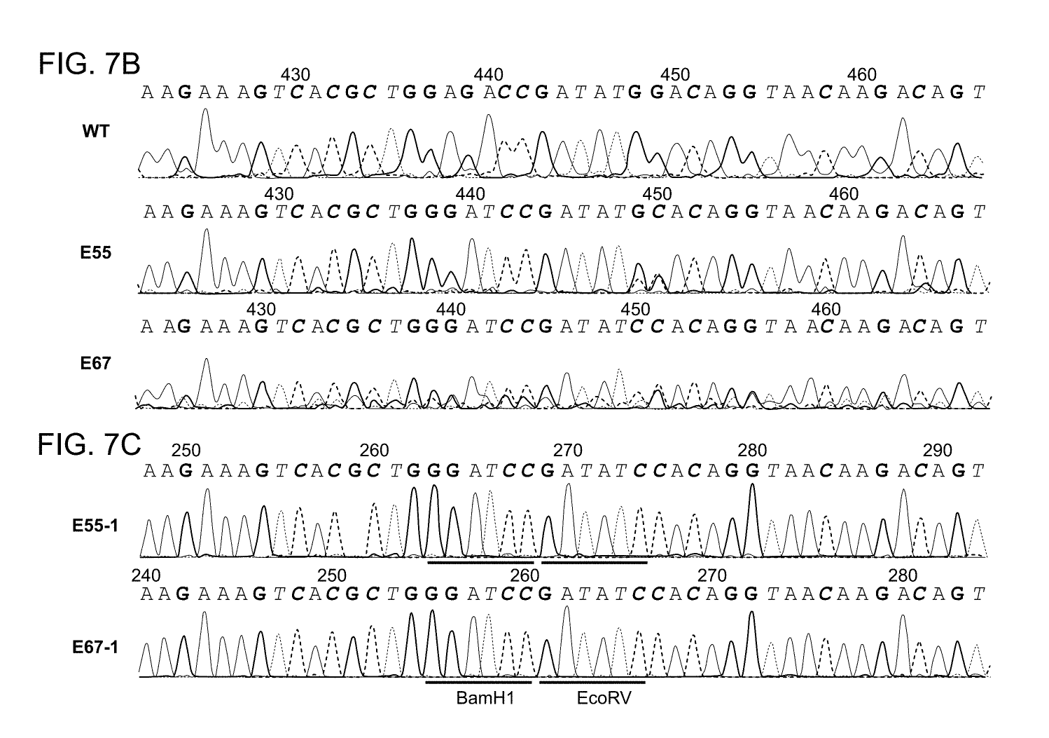

Surprisingly, results obtained from the analysis of the blastocyst samples indicated that NHEJ and KI efficiency were both found to be extremely high after using Cas9 protein in electroporation. Remarkably, using Cas9 protein, even one cycle of electroporation achieved around 70% KI efficiency (FIGS. 7A-7C, and the table immediately below).

Delivery of Cas9 protein through electroporation dramatically
increased the HDR efficiency of Aicda in blastocysts

| Cycles of | Embryos | Total embryos | Percentage | Percentage |
|---|---|---|---|---|
| Electroporation | collected | analyzed | of NHEJ | of KI |
| 1 | 16 | 16 | 33.33% | 66.67% |
| 4 | 16 | 15 | 14.29% | 78.57% |
| 6 | 15 | 13 | 18.18% | 81.82% |
| 8 | 8 | 8 | 16.67% | 83.33% |

To confirm the correct KI, some of the samples were randomly selected for TOPO cloning and DNA sequencing (FIG. 7C). As shown in FIG. 5B, this result was confirmed by the genotyping results of the live born mice. Using one round of electroporation, 60.00% (6/10) of KI efficiency was achieved. Increasing the numbers/cycles of electroporation to 4 times achieved the very similar high efficiency of 71.43% (5/7). Further increase of the number of electroporation to 6 times/cycles and 8 times/cycles yielded 81.33% (5/6) and 100% KI efficiency (5/5), respectively (FIGS. 5A-5C, and Table 1).

TABLE 1
CRISPR/Cas9 mediated genome editing of Aicda gene
locus through electroporation of Cas9 protein

| Times of | | | | | |
|---|---|---|---|---|---|
| Electro- | Embryos | Mice | Total mice | Live born | Percentage |
| poration | transferred | born | analyzed | rate | of NHEJ/KI |
| 1 | 15 | 10 | 10 | 66.67% | 20%/60% |
| 4 | 15 | 7 | 7 | 46.67% | 28.57%/71.43% |
| 6 | 15 | 6 | 6 | 40.00% | 18.67%/81.33% |
| 8 | 15 | 5 | 5 | 33.33% | 0%/100% |

*B6D2F2 embryos were electroporated with a BTX830 electroporator. Following electroporation, the embryos were washed and transferred into pseudopregnant mice. For each live born mouse, PCR products were amplified from genomic DNA and analyzed by RFLP assay and Sanger sequencing.

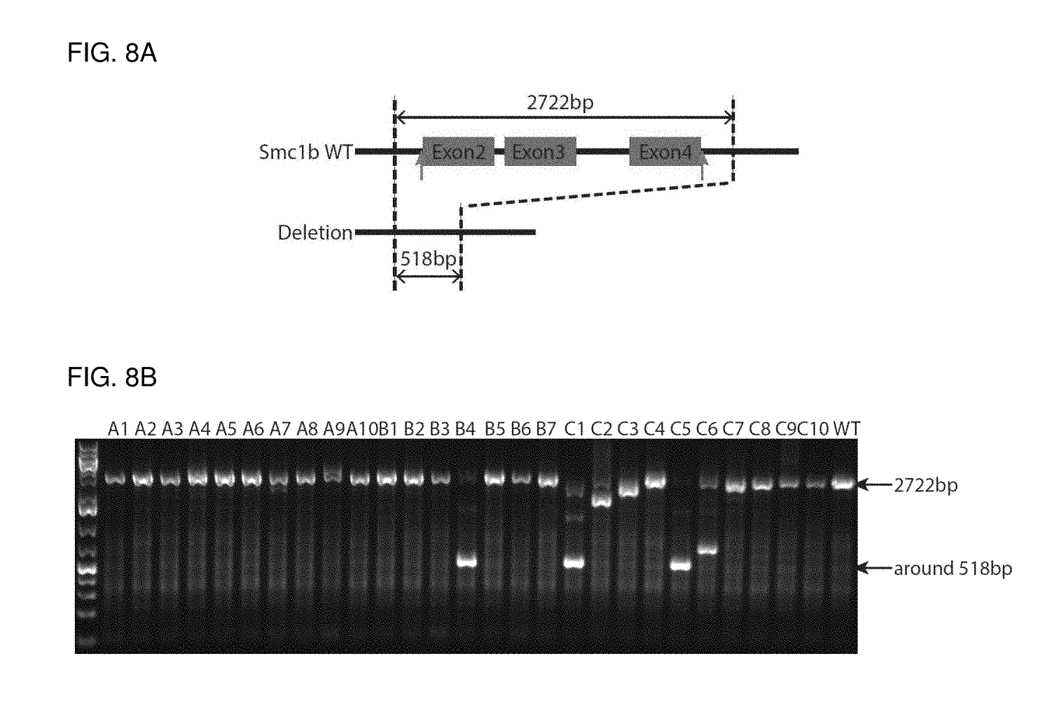

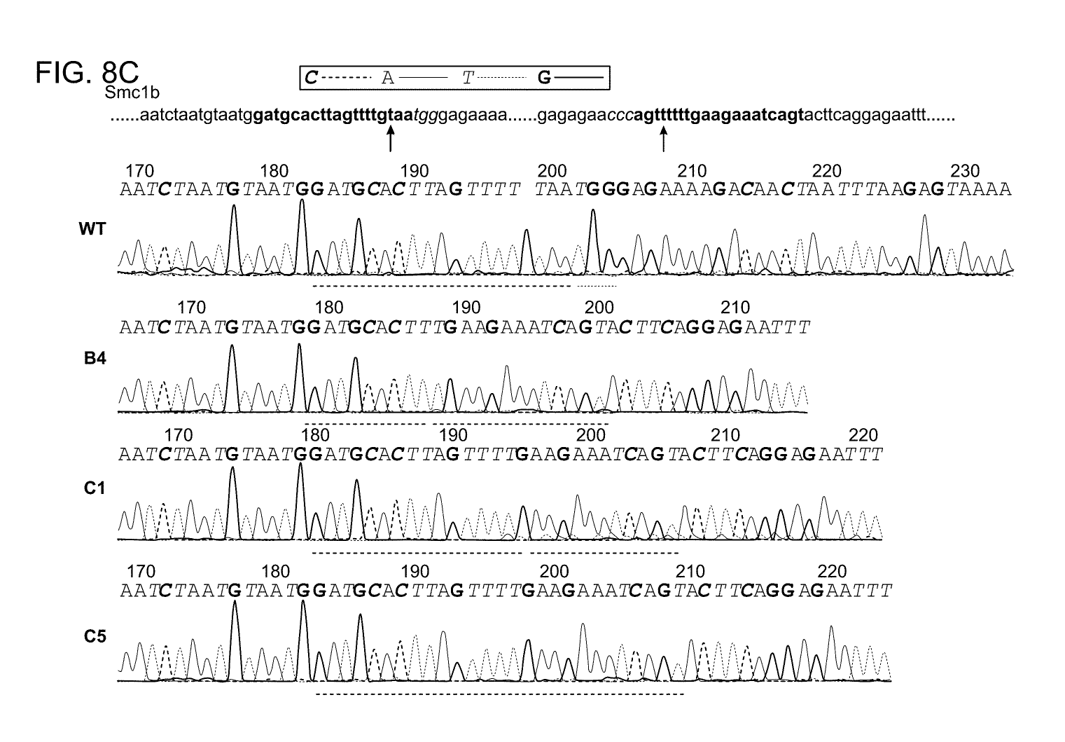

The methods described in the examples above had less than optimal efficiency at creating a 2.2 kb deletion at the Smc1b locus (data not shown). To demonstrate that using Cas9 protein directly in series of electroporation, as described in this example, is efficient to generate large DNA segment deletion at the previously difficult loci, Cas9 protein and two sgRNAs targeting different regions of the Smc1b locus were co-electroporated (FIG. 8A).

Briefly, B6D2F2/J embryos were prepared through IVF (in vitro fertilization) in which GSH (glutathione) was used to weaken the zona pellucida. The fertilized embryos were graded, selected, and washed. The embryos were then electroporated for 1, 4, or 6 times/cycles with a mixture of Cas9 protein (250 ng/μL) and two sgRNAs (300 ng/μL). The electroporated embryos were transferred into the pseudopregnant mice. The tissue samples were taken from the pups and used for purification of genomic DNA and PCR. The PCR products were analyzed through DNA electrophoresis and sequencing.

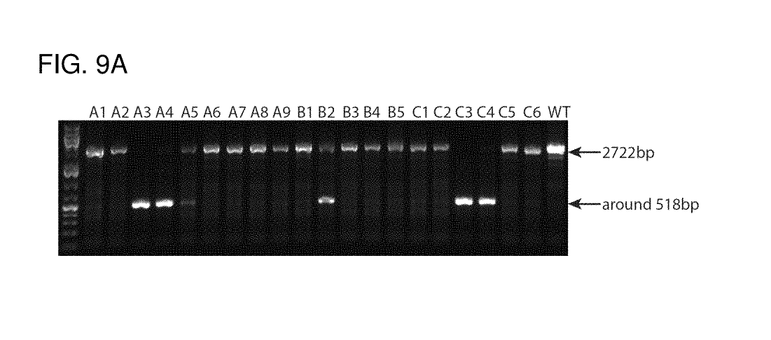

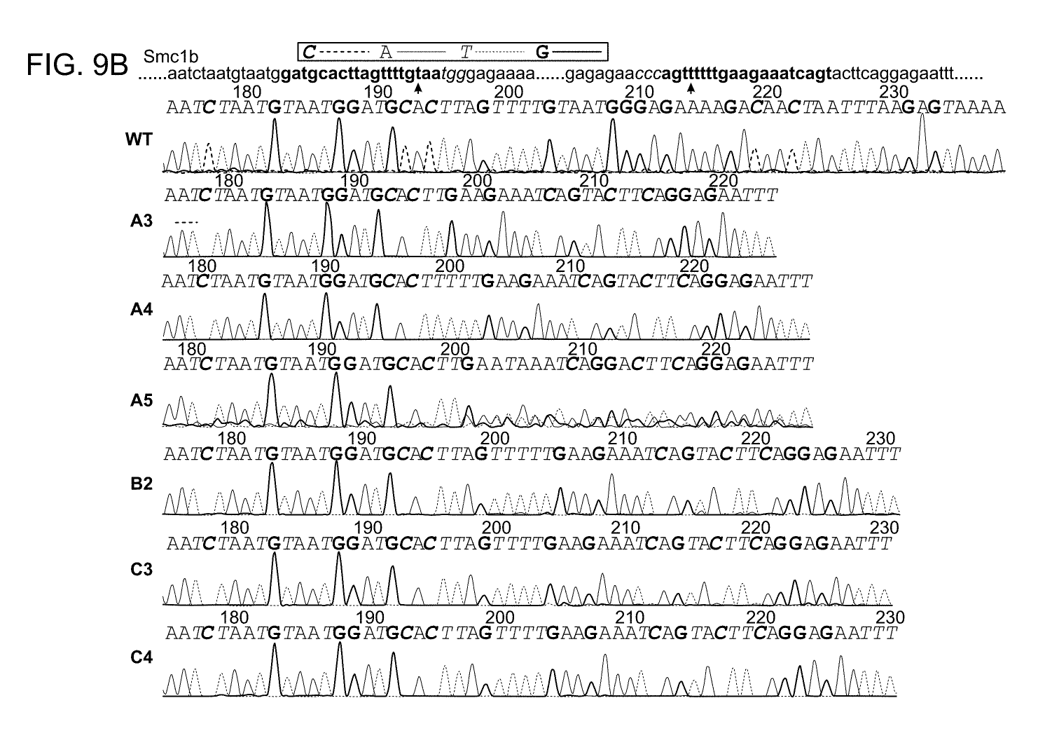

As shown in FIG. 9A, 3/9 mice had the ideal deletion with just one time/cycle of electroporation. A DNA segment around 2200 bp was deleted from the Smc1b locus. Four and 6 series of electroporation yielded 20% (1/5) and 33.34% (2/6) deletion efficiency respectively (FIGS. 9A and 9B, and the table immediately below). The correct deletion was confirmed through Sanger sequencing.

CRISPR/Cas9 mediated large segment deletion of Smc1b through
electroporation of Cas9 protein in IVF generated embryos

| Times of | | | Total | Live | | % of |
|---|---|---|---|---|---|---|
| Electro- | Embryos | Mice | mice | born | % of NHEJ | 2.2 kb |
| poration | transferred | born | analyzed | rate | g1/g2 | deletion |
| 1 | 15 | 9 | 9 | 60.00% | 88.89%/66.67% | 33.33% |
| 4 | 15 | 5 | 5 | 33.33% | 100%/80% | 25% |
| 6 | 15 | 6 | 6 | 40% | 100%/83.33% | 33.33% |
| 8 | 15 | 0 | 0 | 0% | n.a. | n.a. |

B6D2F2 embryos were electroporated with a BTX830 electroporator. Following electroporation, the embryos were washed and transferred into pseudopregnent mice. For each live born mouse, PCR products were amplified from genomic DNA and analyzed by DNA electrophoresis and Sanger sequencing.

To evaluate the modified protocols on inbred strains, the IVF-derived mouse embryos from the C57BL/6NJ strain were isolated and used as the embryo donor. The electroporation was performed using the same protocol as the B6D2F2 hybrid embryos above. Two transfers were performed for each group.

Genotyping results from analysis of the size of PCR products showed that 1 out of 7 mice from four time of electroporation, 3 out of 10 mice from six times of electroporation had large segment deletion (FIG. 8B, Table 2). One cycle electroporation did not work in generating large segment deletion, while it indeed generated NHEJ mutations with high efficiency (Table 2). In addition, series of electroporation did not significantly influence the live-born rate of the inbred strain.

TABLE 2
CRISPR/Cas9 mediated large segment deletion
of Smc1b through electroporation of Cas9 protein in an inbred strain.

| Times of | | Embryos | | Live | Percentage | Percentage |
|---|---|---|---|---|---|---|
| Electropo- | Pseudo- | trans- | Mice | born | of NHEJ | of 2.2 kb |
| ration | ID | ferred | born | rate | g1/g2^{a} | deletion |
| 1 | 1 | 15 | 6 | 33.33% | 80.00%/ | 0% |
| | 2 | 15 | 4 | | 60.00% | |
| 4 | 3 | 15 | 4 | 23.33% | 42.86%/ | 14.29% |
| | 4 | 15 | 3 | | 57.14% | |
| 6 | 5 | 15 | 5 | 33.33% | 100%/ | 30% |
| | 6 | 15 | 5 | | 100% | |

^{a}g1, gRNA1. g2, gRNA2.
* C57BL/6NJ embryos were electroporated with a BTX830 electroporator. Following electroporation, the embryos were washed and transferred into pseudopregnent mice. For each live born mouse, PCR products were amplified from genomic DNA isolated from tail tissue sample and analyzed by DNA electrophoresis and Sanger sequencing.

Overall, the above results demonstrated that Cas9 protein delivery through series of electroporation was highly efficient in generation large DNA segment deletion in the mouse genome.

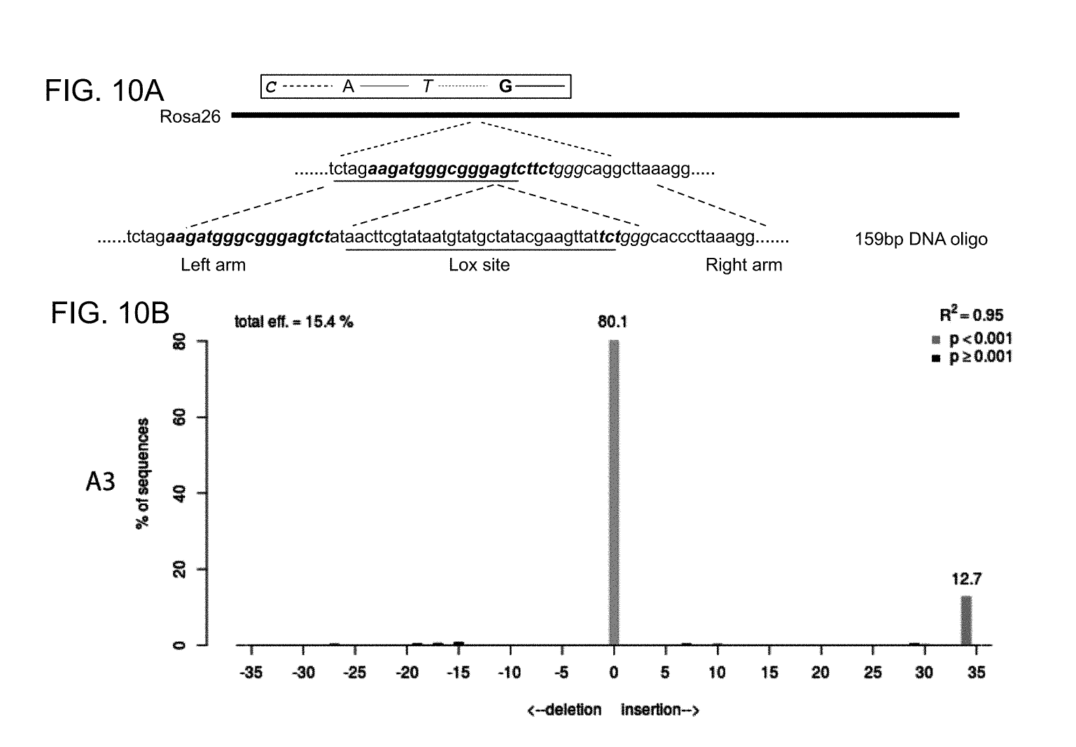

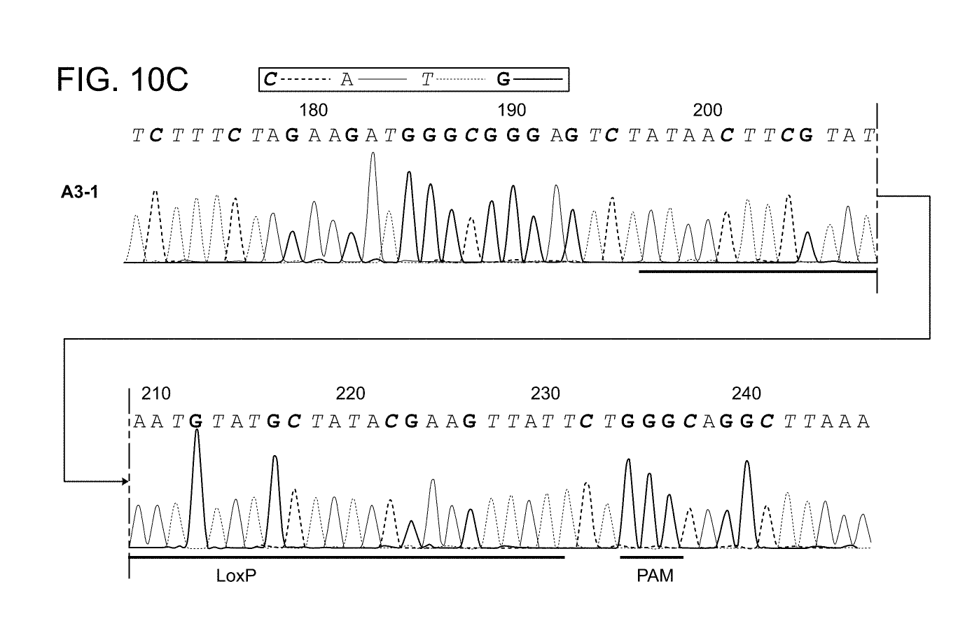

Targeted genome insertion of small DNA segments to generate conditional KO (knock-out) mouse models or to tag a protein or RNA target is widely used in generating mouse models. To test whether delivery of Cas9 protein with series of electroporation is efficient to generate genome insertion of small DNA fragments, IVF embryos were prepared form the C57BL/6NJ strain, and electroporation was performed with a mixture of Cas9 protein (250 ng/μL), sgRNA (300 ng/μL) targeting the Rosa26 locus and DNA oligo with a LoxP site (1000 ng/μL) (FIG. 10A). The embryos were then transferred and live mice were recovered. Tissue samples were taken from the pups and used for purification of genomic DNA and PCR analysis.

TIDE (Tracking of Indels by DEcomposition) analysis of PCR products encompassing the target site showed that 1, 4, and 6 series of electroporation yielded 4 out of 10 mice, 0 out of 5 mice and 2 out of 3 mice that might carry correct LoxP site in Rosa26 locus respectively (Table 3). Those samples were selected and used for TOPO-cloning and Sanger sequencing. The sequencing results confirmed the insertion of correct LoxP site into the Rosa26 locus. Representative images showed the result of TIDE analysis and Sanger sequencing of one of the samples with one time of electroporation in FIGS. 10A-10C.

TABLE 3
CRISPR/Cas9 mediated small segment insertion
of Rosa26 through electroporation of Cas9 protein in an inbred strain.

| Times of | | | | Total | | Percentage |
|---|---|---|---|---|---|---|
| Electro- | Pseudo- | Embryos | Mice | mice | Live born | of |
| poration | ID | transferred | born | analyzed | rate | NHEJ/KI |
| 1 | 1 | 15 | 6 | 6 | 33.33% | 50%/40% |
| | 2 | 15 | 4 | 4 | | |
| 4 | 3 | 15 | 4 | 4 | 20.00% | 83.33%/0% |
| | 4 | 15 | 2 | 2 | | |
| 6 | 5 | 15 | 3 | 3 | 10.00% | 33.33%/ |
| | 6 | 15 | 1bd^{a} | 0 | | 66.67% |

^{a}bd, born-dead.
* C57BL/6NJ embryos were electroporated with a BTX830 electroporator. Following electroporation, the embryos were washed and transferred into pseudopregnent mice. For each live born mouse, PCR products were amplified from genomic DNA isolated from tail tissue sample and analyzed by DNA electrophoresis and Sanger sequencing. bd, born-dead.

These results demonstrated that small DNA fragments can be efficiently inserted into a target region through electroporation using Cas9 protein, including in C57BL/6NJ inbred strain mice.

Data presented in the example demonstrates that, by applying series of electroporation using Cas9 mRNA, sgRNA and donor oligo, efficiency can be improved over that of the methods described in the examples above, though the improved efficiency was still relatively low, e.g., when compared with microinjection.

To further improve the methodology, electroporation was performed using Cas9 protein instead of Cas9 mRNA, and, surprisingly, the KI efficiency was dramatically improved. Although delivery of Cas9 protein using one time of electroporation worked efficiently for genome KI, delivery of Cas9 protein through 6 series of electroporation achieved much higher efficiency in genome modifications (e.g., large DNA segment deletion) with reasonable live born rate, even in inbred strains. For various genetic modifications (precise nucleotide changes, targeted short segment insertion, and large segment deletion) in all three genomic loci, the improved method described herein performed equally well or better than microinjection.

CRISPR/Cas9 system is widely used to generate various animal models (Chapman et al., 2015; Chen et al., 2015; Ma et al., 2014; Niu et al., 2014; Ruan et al., 2015; Yin et al., 2014; Zou et al., 2015). Microinjection is still the main method used to date to deliver CRISPR/Cas9 components into the embryos for most animal species. Compared with microinjection, the improved method developed and described herein, when performed by a novice, is at least as efficient as microinjection performed by a skilled worker, if not better. Microinjection is technically demanding and low in throughput. In addition, it requires significant investment in equipment and operators with years of training and experience. In comparison, the improved methods described herein have obvious advantages including high efficiency, high throughput, and easiness to setup and operate. Thus the method can be used to generate gene edited animal models in most suitable species with high efficiency and ease.

The following general methods and certain reagents were used in conducting the experiments in Example 4, which are intended for illustration purpose only and are non-limiting.

### Preparation of the Reagents for Electroporation

The px330 plasmid was used as the template to amplify the Cas9 coding sequence. T7 promoter sequence was added to the forward primer. In vitro transcription was performed using mMESSAGE mMACHINE T7 ULTRA Transcription kit (Life Technologies).

For sgRNA synthesis, T7 promoter sequence was added to sgRNA sequence through PCR amplification using 2 universal primers (see below) and one primer with various gRNA sequences (see below).

Two universal primers used to prepare IVT DNA
template for all the sgRNAs

| Name | Sequence (5′ to 3′) |
|---|---|
| HW29 | Aaaaaagcaccgactcggtgccactttttcaagttgataacgg |
| | actagccttatttaaacttgctatgctgtttccagcatagctc |
| | ttaaac |
| HW868 | AAAAGCACCGACTCGGTGCC |

Forward primers used to prepare IVT DNA template
for the synthesis of sgRNAs

| sgRNA | Sequence (5′ to 3′) |
|---|---|
| Aicda | taatacgactcactatagAGTCACGCTGGAGACCGATAgt |
| | ttAagagctatgctggaaac |
| Smc1b | taatacgactcactatagGATGCACTTAGTTTTGTAAgtt |
| sgRNA1 | tAagagctatgctggaaac |
| Smc1b | taatacgactcactatagAGTTTTTTGAAGAAATCAGTgt |
| sgRNA2 | ttAagagctatgctggaaac |
| Rosa26 | taatacgactcactatagAAGATGGGCGGGAGTCTTCTgt |
| | ttAagagctatgctggaaac |

The T7-sgRNA PCR product was purified using Qiagen PCR product purification kit and used as the template for IVT. The sgRNAs were synthesized using MEGAshortscript T7 kit (Life Technologies) and purified using MEGAclear kit (Life Technologies).

Single-stranded DNA oligos were ordered as Ultramer DNA oligos from Integrated DNA Technologies (IDT). The DNA oligo sequences were listed below.

DNA oligonucleotides used as DNA donor for HDR-
mediated KI

| Gene | |
|---|---|
| target | Sequence (5′ to 3′) |
| Aicda | tgcttgaagcaagcttcctttggcctaagactttgaggga |
| | gtcaagaaagtcacgctgggatccgatatccacaggtaac |
| | aagacagtctcatctgcttgtgcatgtgctccagtagtgc |
| | tggctgcc |
| Rosa26 | cccttccccctcttccctcgtgatctgcaactccagtctt |
| | tctagaagatgggcgggagtctataacttcgtataatgta |
| | tgctatacgaagttattctgggcaggcttaaaggctaacc |
| | tggtgtgtgggcgttgtcctgcaggggaattgaacagg |

Cas9 mRNA was ordered from Trilink (L-6125). Cas9 protein was ordered from PAN (CP01-50) or Thermo Fisher Scientific (B25641).

To prepare the reagent for electroporation, all the components at the required concentrations were mixed and lyophilized, if the total volume is larger that the final volume for electroporation. The mixture was centrifuged at 20,000 g for 15 min at 4° C. After centrifugation, the supernatant was transferred to a new nuclease free tube. The volume of the final solution was adjusted to the required volume.

To prepare Cas9 RNP (recombinant Cas9 protein complexed with an sgRNA), the electroporation solution including Cas9 protein, and sgRNA with or without DNA oligo were incubated at 37° C. for 15 min. The samples were put on ice and used for electroporation immediately.

### Zygote Isolation, Culture and Transfer

All animal work was approved by the Jackson Laboratory Animal Care and Use Committee and adhered to the standards of Guide for the Care and Use of Laboratory Animals set forth by the NIH.

When generating embryos via natural mating, the mouse embryos were isolated and cultured as described previously (Qin et al., 2015). The IVF was performed following a standard protocol with some minor changes (Byers et al., 2006). In brief, Cook RVF medium was used as the fertilization medium. Cumulus oocyte masses were pre-incubated in 1.0 mM glutathione (GSH) for 30 minutes to improve fertilization rate and weaken the zona pellucida. The embryos were graded for fertilization and viability and deposited into pre-equilibrated microdrops of K-RVCL-50 media in COOK MINC bench top incubator. The PMSG was bought from Prospect or EMD Millipore. The hCG was bought from Prospect or Sigma.

### Zygote Electroporation

Electroporation was performed as described previously (Qin et al., 2015, incorporated by reference). In brief, zygotes were treated with the acidic Tyrode's solution (T1788, Sigma-Aldrich) for 10 seconds and washed extensively in pre-warmed M2 media. The embryos produced by IVF were not treated with the acidic Tyrode's solution. To weaken the zona pellucida, they were previously treated with GSH before fertilization in the modified IVF protocol.

Zygotes were then placed in 10 μL drops of Opti-MEM media (P/N 31985, Gibco). 10 μL of solution including the Cas9 mRNA or Cas9 protein/sgRNA/donor was mixed with the Opti-MEM drops with the embryos and deposited into a 1 mm electroporation cuvette (P/N 45-0124, Harvard Apparatus).

Electroporation was performed in an ECM830 Square Wave Electroporation System (BTX Harvard Apparatus). The electroporation setting for each cycle is the same for all the experiments using 1 ms pulse duration and two pulses with 100 ms pulse interval at 30V. Following electroporation, a pre-warmed 100 μL aliquot of M2 media was deposited into the cuvette with a sterile plastic pipette to recover the embryos. The zygotes were removed from the cuvette and washed in pre-warmed M2 media. Embryos were then cultured in vitro to blastocysts in a MINC benchtop incubator (COOK; 37° C., 5% CO₂ and 5% O₂/Nitrogen) or transferred into pseudopregnant female mice (CByB6F1/J). For the IVF embryos, they were cultured to the two-cell stage and then transferred.

### RFLP Analysis and Sanger Sequencing

Genomic DNA from tissue or embryos was extracted using alkaline lysis as described before (Qin et al., 2015). PCRs with specific primers were performed under the following conditions: 98° C. for 5 min; 34×(98° C. for 30 s, 58° C. (or other annealing temperature) for 30 s, 68° C. for 30 s); 68° C. for 2 min; hold at 4° C. About 4 μL of PCR products were digested with restriction enzymes and separated on a agarose gel (1.0%) with GelRed (Biotium). PCR products were sequenced and cloned into the pCR4 blunt-end vector from the Zero Blunt TOPO® PCR Cloning Kit (Invitrogen) to identify the correct single clones. The sequences of the individual clones were determined by Sanger sequencing.

### REFERENCES

Aida, T., Chiyo, K., Usami, T., Ishikubo, H., Imahashi, R., Wada, Y., Tanaka, K. F., Sakuma, T., Yamamoto, T., and Tanaka, K. (2015). Cloning-free CRISPR/Cas system facilitates functional cassette knock-in in mice. Genome Biol 16, 87.
Blasco, R. B., Karaca, E., Ambrogio, C., Cheong, T. C., Karayol, E., Minero, V. G., Voena, C., and Chiarle, R. (2014). Simple and rapid in vivo generation of chromosomal rearrangements using CRISPR/Cas9 technology. Cell Rep 9, 1219-1227.
Bogdanove, A. J., and Voytas, D. F., “TAL effectors: customizable proteins for DNA targeting,” Science, 333:1843-1846 (2011).
Brown, A. J., Fisher, D. A., Kouranova, E., McCoy, A., Forbes, K., Wu, Y., Henry, R., Ji, D., Chambers, A., Warren, J., et al., “Whole-rat conditional gene knockout via genome editing,” Nat. Methods, 10:638-640 (2013).
Byers, S. L., Payson, S. J., and Taft, R. A. (2006). Performance of ten inbred mouse strains following assisted reproductive technologies (ARTs). Theriogenology 65, 1716-1726.
Capecchi, M. R., “Gene targeting in mice: functional analysis of the mammalian genome for the twenty-first century,” Nat. Rev. Genet., 6:507-512 (2005). Chapman, K. M., Medrano, G. A., Jaichander, P., Chaudhary, J., Waits, A. E., Nobrega, M. A., Hotaling, J. M., Ober, C., and Hamra, F. K. (2015). Targeted Germline Modifications in Rats Using CRISPR/Cas9 and Spermatogonial Stem Cells. Cell Rep 10, 1828-1835.
Carbery, I. D., Ji, D., Harrington, A., Brown, V., Weinstein, E. J., Liaw, L., and Cui, X., “Targeted genome modification in mice using zinc-finger nucleases,” Genetics, 186:451-459 (2010).
Chang, N., Sun, C., Gao, L., Zhu, D., Xu, X., Zhu, X., Xiong, J. W., and Xi, J. J., “Genome editing with RNA-guided Cas9 nuclease in Zebrafish embryos,” Cell Res., 23:465-472 (2013).
Chen, S., Sanjana, N. E., Zheng, K., Shalem, O., Lee, K., Shi, X., Scott, D. A., Song, J., Pan, J. Q., Weissleder, R., et al. (2015). Genome-wide CRISPR screen in a mouse model of tumor growth and metastasis. Cell 160, 1246-1260.
Cong, L., Ran, F. A., Cox, D., Lin, S. L., Barretto, R., Habib, N., Hsu, P. D., Wu, X. B., Jiang, W. Y., Marraffini, L. A., et al. (2013). Multiplex Genome Engineering Using CRISPR/Cas Systems. Science 339, 819-823.
Cui, X., Ji, D., Fisher, D. A., Wu, Y., Briner, D. M., and Weinstein, E. J., “Targeted integration in rat and mouse embryos with zinc-finger nucleases,” Nat. Biotechnol., 29:64-67 (2011).
Geurts, A. M., Cost, G. J., Freyvert, Y., Zeitler, B., Miller, J. C., Choi, V. M., Jenkins, S. S., Wood, A., Cui, X., Meng, X., et al. “Knockout rats via embryo microinjection of zinc-finger nucleases,” Science, 325:433 (2009).
Hai, T., Teng, F., Guo, R., Li, W., and Zhou, Q. (2014). One-step generation of knockout pigs by zygote injection of CRISPR/Cas system. Cell Res 24, 372-375.
Hashimoto, M., and Takemoto, T. (2015). Electroporation enables the efficient mRNA delivery into the mouse zygotes and facilitates CRISPR/Cas9-based genome editing. Sci Rep 5, 11315.
Heckl, D., Kowalczyk, M. S., Yudovich, D., Belizaire, R., Puram, R. V., McConkey, M. E., Thielke, A., Aster, J. C., Regev, A., and Ebert, B. L. (2014). Generation of mouse models of myeloid malignancy with combinatorial genetic lesions using CRISPR-Cas9 genome editing. Nat Biotechnol 32, 941-946.
Hsu, P. D., Lander, E. S., and Zhang, F. (2014). Development and applications of CRISPR-Cas9 for genome engineering. Cell 157, 1262-1278.
Hwang, W. Y., Fu, Y., Reyon, D., Maeder, M. L., Tsai, S. Q., Sander, J. D., Peterson, R. T., Yeh, J. R., and Joung, J. K., “Efficient genome editing in zebrafish using a CRISPR-Cas system,” Nat. Biotechnol., 31:227-229 (2013).
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Li, D., Qiu, Z., Shao, Y., Chen, Y., Guan, Y., Liu, M., Li, Y., Gao, N., Wang, L., Lu, X., et al., “Heritable gene targeting in the mouse and rat using a CRISPR-Cas system,” Nat. Biotechnol., 31:681-683 (2013a).
Li, W., Teng, F., Li, T., and Zhou, Q., “Simultaneous generation and germline transmission of multiple gene mutations in rat using CRISPR-Cas systems,” Nat. Biotechnol., 31:684-686 (2013b).
Liang, X., Potter, J., Kumar, S., Zou, Y., Quintanilla, R., Sridharan, M., Carte, J., Chen, W., Roark, N., Ranganathan, S., et al. (2015). Rapid and highly efficient mammalian cell engineering via Cas9 protein transfection. J Biotechnol 208, 44-53.
Ma, Y., Zhang, X., Shen, B., Lu, Y., Chen, W., Ma, J., Bai, L., Huang, X., and Zhang, L. (2014). Generating rats with conditional alleles using CRISPR/Cas9. Cell Res 24, 122-125.
Maddalo, D., Manchado, E., Concepcion, C. P., Bonetti, C., Vidigal, J. A., Han, Y. C., Ogrodowski, P., Crippa, A., Rekhtman, N., de Stanchina, E., et al. (2014). In vivo engineering of oncogenic chromosomal rearrangements with the CRISPR/Cas9 system. Nature 516, 423-427.
Mali, P., Esvelt, K. M., and Church, G. M. (2013a). Cas9 as a versatile tool for engineering biology. Nat Methods 10, 957-963.
Mali, P., Yang, L., Esvelt, K. M., Aach, J., Guell, M., DiCarlo, J. E., Norville, J. E., and Church, G. M. (2013b). RNA-guided human genome engineering via Cas9. Science 339, 823-826.
Meyer, M., de Angelis, M. H., Wurst, W., and Kuhn, R., “Gene targeting by homologous recombination in mouse zygotes mediated by zinc-finger nucleases,” Proc. Natl. Acad. Sci. USA, 107:15022-15026 (2010).
Niu, Y., Shen, B., Cui, Y., Chen, Y., Wang, J., Wang, L., Kang, Y., Zhao, X., Si, W., Li, W., et al. (2014). Generation of gene-modified cynomolgus monkey via Cas9/RNA-mediated gene targeting in one-cell embryos. Cell 156, 836-843.
Perdomini, M., Belbellaa, B., Monassier, L., Reutenauer, L., Messaddeq, N., Cartier, N., Crystal, R. G., Aubourg, P., and Puccio, H. (2014). Prevention and reversal of severe mitochondrial cardiomyopathy by gene therapy in a mouse model of Friedreich's ataxia. Nat Med 20, 542-547.
Platt, R. J., Chen, S., Zhou, Y., Yim, M. J., Swiech, L., Kempton, H. R., Dahlman, J. E., Parnas, O., Eisenhaure, T. M., Jovanovic, M., et al. (2014). CRISPR-Cas9 knockin mice for genome editing and cancer modeling. Cell 159, 440-455.
Sung, Y. H., Baek, I. J., Kim, D. H., Jeon, J., Lee, J., Lee, K., Jeong, D., Kim, J. S., and Lee, H. W., “Knockout mice created by TALEN-mediated gene targeting,” Nat. Biotechnol., 31:23-24 (2013).
Tesson, L., Usal, C., Menoret, S., Leung, E., Niles, B. J., Remy, S., Santiago, Y., Vincent, A. I., Meng, X., Zhang, L., et al., “Knockout rats generated by embryo microinjection of TALENs,” Nat. Biotechnol., 29:695-696 (2011).
Urnov, F. D., Rebar, E. J., Holmes, M. C., Zhang, H. S., and Gregory, P. D., “Genome editing with engineered zinc finger nucleases,” Nat. Rev. Genet., 11:636-646 (2010).
Qin, W., Dion, S. L., Kutny, P. M., Zhang, Y., Cheng, A., Jillette, N. L., Malhotra, A., Geurts, A. M., Chen, Y. G., and Wang, H. (2015). Efficient CRISPR/Cas9-Mediated Genome Editing in Mice by Zygote Electroporation of Nuclease. Genetics.
Ruan, J., Li, H., Xu, K., Wu, T., Wei, J., Zhou, R., Liu, Z., Mu, Y., Yang, S., Ouyang, H., et al. (2015). Highly efficient CRISPR/Cas9-mediated transgene knockin at the H11 locus in pigs. Sci Rep 5, 14253.
Schumann, K., Lin, S., Boyer, E., Simeonov, D. R., Subramaniam, M., Gate, R. E., Haliburton, G. E., Ye, C. J., Bluestone, J. A., Doudna, J. A., et al. (2015). Generation of knock-in primary human T cells using Cas9 ribonucleoproteins. Proc Natl Acad Sci USA 112, 10437-10442.
Wang, H., Yang, H., Shivalila, C. S., Dawlaty, M. M., Cheng, A. W., Zhang, F., and Jaenisch, R., “One-step generation of mice carrying mutations in multiple genes by CRISPR/Cas-mediated genome engineering,” Cell, 153:910-918 (2013).
Xue, W., Chen, S., Yin, H., Tammela, T., Papagiannakopoulos, T., Joshi, N. S., Cai, W., Yang, G., Bronson, R., Crowley, D. G., et al. (2014). CRISPR-mediated direct mutation of cancer genes in the mouse liver. Nature 514, 380-384.
Yang, H., Wang, H., Shivalila, C. S., Cheng, A. W., Shi, L., and Jaenisch, R. (2013). One-step generation of mice carrying reporter and conditional alleles by CRISPR/Cas-mediated genome engineering. Cell 154, 1370-1379.
Yang, L., Guell, M., Niu, D., George, H., Lesha, E., Grishin, D., Aach, J., Shrock, E., Xu, W., Poci, J., et al. (2015). Genome-wide inactivation of porcine endogenous retroviruses (PERVs). Science 350, 1101-1104.
Yin, H., Xue, W., Chen, S., Bogorad, R. L., Benedetti, E., Grompe, M., Koteliansky, V., Sharp, P. A., Jacks, T., and Anderson, D. G. (2014). Genome editing with Cas9 in adult mice corrects a disease mutation and phenotype. Nat Biotechnol 32, 551-553.
Zou, Q., Wang, X., Liu, Y., Ouyang, Z., Long, H., Wei, S., Xin, J., Zhao, B., Lai, S., Shen, J., et al. (2015). Generation of gene-target dogs using CRISPR/Cas9 system. J Mol Cell Biol.
All references cited herein are incorporated herein by reference.

1. A method of generating a genetically modified non-human mammal, the method comprising introducing, via electroporation, a CRISPR-Cas system comprising a Type-II Cas9 protein, into a zygote of a non-human mammal to modify the genome of the non-human mammal, wherein said electroporation comprises 4-10 cycles.

2. The method of claim 1, wherein said electroporation comprises 4-8 cycles, or 6 cycles.

3. The method of claim 1, wherein each cycle of said electroporation is independently carried out at 20-40 volts (e.g., 25-35 volts, or 30 volts, or 25, 26, 27, 28, 29, 30, 31, 32, 33, 34, or 35 volts).

4. The method of claim 1, wherein each cycle of said electroporation is independently carried out using a pulse duration of 1-2 ms (e.g., 1.0 or 1.5 ms).

5. The method of claim 1, wherein each cycle of said electroporation is independently carried out using 1-3 pulses (e.g., 2 pulses).

6. The method of claim 1, wherein each cycle of said electroporation is independently carried out using a pulse interval of 100-1000 ms (e.g., 100 ms, or 125-130 ms).

7. The method of claim 1, wherein said electroporation consists of 6 cycles, and each cycle of said electroporation is carried out using the settings of: 30 volts, 1.0 ms pulse duration, 2 pulses, and 100 ms pulse interval.

8. The method of claim 1, wherein said Cas9 protein is electroporated at a final concentration of 150-350 ng/μL (e.g., 150 ng/μL, 200 ng/μL, 250 ng/μL, 300 ng/μL, or 350 ng/μL).

9. The method of claim 1, wherein said CRISPR-Cas system comprises a single guide RNA (sgRNA) that hybridizes to a target sequence in the genome of the non-human mammal, and wherein the sgRNA is electroporated at a final concentration of 300 ng/μL (e.g., 200 ng/μL, 250 ng/μL, 300 ng/μL, 350 ng/μL, or 400 ng/μL).

10. The method of claim 1, wherein said Cas9 protein is electroporated into the zygote with a donor polynucleotide.

11. The method of claim 10, wherein said donor polynucleotide is a linear or circular double stranded DNA molecule.

12.-13. (canceled)

14. The method of claim 1, wherein the method achieves a knock-in (KI) efficiency of at least 60%, 65%, 70%, 75%, 80%, 85%, 90%, 95% or more (e.g., 100%).

15. The method of claim 1, wherein the method achieves a NHEJ efficiency of at least 80%, 85%, 90%, 95%, 97%, or 100%.

16. The method of claim 1, wherein the method achieves deletion of an endogenous genomic region of at least 1 kb, 1.5 kb, 2 kb, 2.5 kb, 3 kb or more, in IVF (in vireo fertilization)-generated embryos at a rate of at least 10%, 15%, 20%, 25%, 30%, 35%, 40% or more.

17. The method of claim 1, wherein more than one zygotes are simultaneously electroporated.

18.-20. (canceled)

21. The method of claim 1, wherein the zygote is pre-treated to weaken Zona Pellucida prior to electroporation.

22. The method of claim 21, wherein the zygote is pre-treated in Acidic Tyrode's solution (AT) for 5-15 seconds, or 10 seconds.

23.-24. (canceled)

25. The method of claim 1, further comprising transferring the electroporated zygote to a pseudopregnant host mammal and allowing the electroporated zygote to develop into a live-born genetically modified mammal.

26. The method of claim 1, wherein the non-human mammal is a primate (e.g., marmoset, rhesus monkey, chimpanzee), a rodent (e.g., mouse, rat, gerbil, Guinea pig, hamster, cotton rat, naked mole rat), a rabbit, a livestock mammal (e.g., goat, sheep, pig, cow, cattle, horse, camelid), a pet mammal (e.g., dog, cat), a marsupial, or an outbred or a random bred population thereof.

27. (canceled)

28. A genetically modified non-human mammal generated by any one of the methods of claim 1.


SEQUENCE LISTING (64 sequences)

SEQ ID NO: 1 - protein, 11 aa, Human immunodeficiency virus
YGRKKRRQRRR

SEQ ID NO: 2 - protein, 12 aa, Unknown
  source: /note=“Description of Unknown Exemplary PTD peptide”
RRQRRTSKLMKR

SEQ ID NO: 3 - protein, 27 aa, Unknown
  source: /note=“Description of Unknown Transportan peptide”
GWTLNSAGYLLGKINLKALAALAKKIL

SEQ ID NO: 4 - protein, 33 aa, Unknown
  source: /note=“Description of Unknown Exemplary PTD peptide”
KALAWEAKLAKALAKALAKHLAKALAKALKCEA

SEQ ID NO: 5 - protein, 16 aa, Unknown
  source: /note=“Description of Unknown Exemplary PTD peptide”
RQIKIWFQNRRMKWKK

SEQ ID NO: 6 - protein, 9 aa, Human immunodeficiency virus
RKKRRQRRR

SEQ ID NO: 7 - protein, 8 aa, Unknown
  source: /note=“Description of Unknown Exemplary PTD peptide”
RKKRRQRR

SEQ ID NO: 8 - protein, 11 aa, Unknown
  source: /note=“Description of Unknown Exemplary PTD peptide”
YARAAARQARA

SEQ ID NO: 9 - protein, 11 aa, Unknown
  source: /note=“Description of Unknown Exemplary PTD peptide”
THRLPRRRRRR

SEQ ID NO: 10 - protein, 11 aa, Unknown
  source: /note=“Description of Unknown Exemplary PTD peptide”
GGRRARRRRRR

SEQ ID NO: 11 - RNA, 12 nt, Artificial Sequence
  source: /note=“Description of Artificial Sequence Synthetic oligonucleotide”
guuuuagagcua

SEQ ID NO: 12 - RNA, 26 nt, Artificial Sequence
  source: /note=“Description of Artificial Sequence Synthetic oligonucleotide”
uagcaaguuaaaauaaggcuaguccg

SEQ ID NO: 13 - DNA, 56 bp, Mus sp.
gaccaagtgtggctgctgtcagggagctcatggagactaggtgaggaactctgctt

SEQ ID NO: 14 - DNA, 56 bp, Mus sp.
gaccaagtgtggctgctgtcagggagcccatggagactaggtgaggaactctgctt

SEQ ID NO: 15 - DNA, 56 bp, Mus sp.
gaccaagtgtggctgctgtcagggagctaatggagactaggtgaggaactctgctt

SEQ ID NO: 16 - DNA, 56 bp, Mus sp.
agaaacacgtgaaagtgccaacagatatccaggctgcagaatcggagaaccacgcc

SEQ ID NO: 17 - DNA, 54 bp, Mus sp.
agaaacacgtgaaagtgccaacagatccaggctgcagaatcggagaaccacgcc

SEQ ID NO: 18 - DNA, 51 bp, Mus sp.
agaaacacgtgaaagtgccaacagcaggctgcagaatcggagaaccacgcc

SEQ ID NO: 19 - DNA, 50 bp, Mus sp.
cagaaacacgtgaaagtgccaacagattcaggaagccgaaaaagagaacc

SEQ ID NO: 20 - DNA, 49 bp, Mus sp.
cagaaacacgtgaaagtgccaacagatatccaggctgcagaatcggaga

SEQ ID NO: 21 - DNA, 50 bp, Mus sp.
cagaaacacgtgaaagtgccaacaggctgaataggagaacaacccccccc

SEQ ID NO: 22 - DNA, 50 bp, Mus sp.
cagaaacacgtgaaagtgccaacagatatccaggctgcagaatcggagaa

SEQ ID NO: 23 - DNA, 50 bp, Mus sp.
cagaaacacgtgaaagtgccaacagatatccaggctgcaaaatcggaaaa

SEQ ID NO: 24 - DNA, 50 bp, Mus sp.
cagaaacacgtgaaagtgccaacaggttgcccagtcggagaaccacgcca

SEQ ID NO: 25 - DNA, 76 bp, Mus sp.
agaaacacgtgaaagtgccaacaggctgcagaatcggagaaccacgcccgagctgcagagcctcaagcaaccaaaa

SEQ ID NO: 26 - DNA, 86 bp, Mus sp.
agaaacacgtgaaagtgccaacagatacatccaggctgcagaatcggagaaccacgcccgagctgcagagcctcaagcaaccaaaa

SEQ ID NO: 27 - DNA, 81 bp, Mus sp.
agaaacacgtgaaagtgccaacagatatatctgcagaatcggagaaccacgcccgagctgcagagcctcaagcaaccaaaa

SEQ ID NO: 28 - DNA, 73 bp, Mus sp.
agaaacacgtgaaagtgccaggctgcagaatcggagaaccacgcccgagctgcagagcctcaagcaaccaaaa

SEQ ID NO: 29 - DNA, 84 bp, Mus sp.
agaaacacgtgaaagtgccaacagatacccaggctgcagaatcggagaaccacgcccgagctgcagagcctcaagcaaccaaaa

SEQ ID NO: 30 - DNA, 84 bp, Mus sp.
agaaacacgtgaaagtgccaacagatatccaggctgcagaatcggagaaccacgcccgagctgcagagcctcaagcaaccaaaa

SEQ ID NO: 31 - DNA, 50 bp, Mus sp.
cagaaacacgtgaaagtgccaacagatttccagggtgaacaaccggagaa

SEQ ID NO: 32 - DNA, 50 bp, Mus sp.
cagaaacacgtgaaagtgccaacagaattccaggctgcagaatcggagaa

SEQ ID NO: 33 - DNA, 50 bp, Mus sp.
ttctccgattctgcagcctggaattctgttggcactttcacgtgtttctg

SEQ ID NO: 34 - DNA, 50 bp, Mus sp.
cagaaacacgtgaaagtgccaacaggatgccgagttggagaaccgcacac

SEQ ID NO: 35 - protein, 50 aa, Unknown
  source: /note=“Description of Unknown Exemplary PTD peptide”
  MISC_FEATURE (1)..(50): /note=“This sequence may encompass 3-50 residues”
RRRRRRRRRRRRRRRRRRRRRRRRRRRRRRRRRRRRRRRRRRRRRRRRRR

SEQ ID NO: 36 - protein, 9 aa, Unknown
  source: /note=“Description of Unknown Exemplary CPP peptide”
RRRRRRRRR

SEQ ID NO: 37 - protein, 9 aa, Unknown
  source: /note=“Description of Unknown Exemplary CPP peptide”
EEEEEEEEE

SEQ ID NO: 38 - DNA, 13 bp, Artificial Sequence
  source: /note=“Description of Artificial Sequence Synthetic oligonucleotide”
gagaccgatatgg

SEQ ID NO: 39 - DNA, 92 bp, Artificial Sequence
  source: /note=“Description of Artificial Sequence Synthetic primer”
aaaaaagcaccgactcggtgccactttttcaagttgataacggactagccttatttaaacttgctatgctgtttccagcatagctcttaaac

SEQ ID NO: 40 - DNA, 20 bp, Artificial Sequence
  source: /note=“Description of Artificial Sequence Synthetic primer”
aaaagcaccgactcggtgcc

SEQ ID NO: 41 - DNA, 60 bp, Artificial Sequence
  source: /note=“Description of Artificial Sequence Synthetic primer”
taatacgactcactatagagtcacgctggagaccgatagtttaagagctatgctggaaac

SEQ ID NO: 42 - DNA, 59 bp, Artificial Sequence
  source: /note=“Description of Artificial Sequence Synthetic primer”
taatacgactcactataggatgcacttagttttgtaagtttaagagctatgctggaaac

SEQ ID NO: 43 - DNA, 60 bp, Artificial Sequence
  source: /note=“Description of Artificial Sequence Synthetic primer”
taatacgactcactatagagttttttgaagaaatcagtgtttaagagctatgctggaaac

SEQ ID NO: 44 - DNA, 60 bp, Artificial Sequence
  source: /note=“Description of Artificial Sequence Synthetic primer”
taatacgactcactatagaagatgggcgggagtcttctgtttaagagctatgctggaaac

SEQ ID NO: 45 - DNA, 128 bp, Artificial Sequence
  source: /note=“Description of Artificial Sequence Synthetic polynucleotide”
tgcttgaagcaagcttcctttggcctaagactttgagggagtcaagaaagtcacgctgggatccgatatccacaggtaacaagacagtctcatctgcttgtgcatgtgctccagtagtgctggctgcc

SEQ ID NO: 46 - DNA, 158 bp, Artificial Sequence
  source: /note=“Description of Artificial Sequence Synthetic polynucleotide”
cccttccccctcttccctcgtgatctgcaactccagtctttctagaagatgggcgggagtctataacttcgtataatgtatgctatacgaagttattctgggcaggcttaaaggctaacctggtgtgtgggcgttgtcctgcaggggaattgaacagg

SEQ ID NO: 47 - DNA, 45 bp, Mus sp.
aagaaagtcacgctggagaccgatatggacaggtaacaagacagt

SEQ ID NO: 48 - DNA, 45 bp, Mus sp.
aagaaagtcacgctgggatccgatatccacaggtaacaagacagt

SEQ ID NO: 49 - DNA, 45 bp, Mus sp.
aagaaagtcacgctggaatccgatatccacaggtaacaagacagt

SEQ ID NO: 50 - DNA, 45 bp, Mus sp.
aagaaagtcacgctggaaaccgatatgcacaggtaacaagacagt

SEQ ID NO: 51 - DNA, 45 bp, Mus sp.
aagaaagtcacgctgggatccgatatgcacaggtaacaagacagt

SEQ ID NO: 52 - DNA, 84 bp, Mus sp.
agaaacacgtgaaagtgccaacagatacccagtcggcagaatcggagaaccacgcccgagctgcagagcctcaagcaaccaaaa

SEQ ID NO: 53 - DNA, 43 bp, Mus sp.
aatctaatgtaatggatgcacttagttttgtaatgggagaaaa

SEQ ID NO: 54 - DNA, 45 bp, Mus sp.
gagagaacccagttttttgaagaaatcagtacttcaggagaattt

SEQ ID NO: 55 - DNA, 65 bp, Mus sp.
aatctaatgtaatggatgcacttagttttgtaatgggagaaaagacaactaatttaagagtaaaa

SEQ ID NO: 56 - DNA, 51 bp, Mus sp.
aatctaatgtaatggatgcactttgaagaaatcagtacttcaggagaattt

SEQ ID NO: 57 - DNA, 56 bp, Mus sp.
aatctaatgtaatggatgcacttagttttgaagaaatcagtacttcaggagaattt

SEQ ID NO: 58 - DNA, 50 bp, Mus sp.
aatctaatgtaatggatgcacttgaagaaatcagtacttcaggagaattt

SEQ ID NO: 59 - DNA, 53 bp, Mus sp.
aatctaatgtaatggatgcactttttgaagaaatcagtacttcaggagaattt

SEQ ID NO: 60 - DNA, 50 bp, Mus sp.
aatctaatgtaatggatgcacttgaataaatcaggacttcaggagaattt

SEQ ID NO: 61 - DNA, 57 bp, Mus sp.
aatctaatgtaatggatgcacttagtttttgaagaaatcagtacttcaggagaattt

SEQ ID NO: 62 - DNA, 40 bp, Mus sp.
tctagaagatgggcgggagtcttctgggcaggcttaaagg

SEQ ID NO: 63 - DNA, 74 bp, Mus sp.
tctagaagatgggcgggagtctataacttcgtataatgtatgctatacgaagttattctgggcaggcttaaagg

SEQ ID NO: 64 - DNA, 76 bp, Mus sp.
tctttctagaagatgggcgggagtctataacttcgtataatgtatgctatacgaagttattctgggcaggcttaaa